SEQUENCE LISTING (52 sequences)

SEQ ID NO: 1 - protein, 501 aa, Human papilloma virus type 11 (HPV11)
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 2 - DNA, 1503 bp, artificial sequence
  cDNA
atgtggcgcccgagcgatagcaccgtgtatgtgccgccgccgaacccggtgagcaaagtggtggcgaccgatgcgtatgtgaaacgcaccaacattttttatcatgcgagcagcagccgcctgctggcggtgggccatccgtattatagcattaaaaaagtgaacaaaaccgtggtgccgaaagtgagcggctatcagtatcgcgtgtttaaagtggtgctgccggatccgaacaaatttgcgctgccggatagcagcctgtttgatccgaccacccagcgcctggtgtgggcgtgcaccggcctggaagtgggccgcggccagccgctgggcgtgggcgtgagcggccatccgctgctgaacaaatatgatgatgtggaaaacagcggcggctatggcggcaacccgggccaggataaccgcgtgaacgtgggcatggattataaacagacccagctgtgcatggtgggctgcgcgccgccgctgggcgaacattggggcaaaggcacccagtgcagcaacaccagcgtgcagaacggcgattgcccgccgctggaactgattaccagcgtgattcaggatggcgatatggtggataccggctttggcgcgatgaactttgcggatctgcagaccaacaaaagcgatgtgccgctggatatttgcggcaccgtgtgcaaatatccggattatctgcagatggcggcggatccgtatggcgatcgcctgtttttttatctgcgcaaagaacagatgtttgcgcgccatttttttaaccgcgcgggcaccgtgggcgaaccggtgccggatgatctgctggtgaaaggcggcaacaaccgcagcagcgtggcgagcagcatttatgtgcataccccgagcggcagcctggtgagcagcgaagcgcagctgtttaacaaaccgtattggctgcagaaagcgcagggccataacaacggcatttgctggggcaaccatctgtttgtgaccgtggtggataccacccgcagcaccaacatgaccctgtgcgcgagcgtgagcaaaagcgcgacctataccaacagcgattataaagaatatatgcgccatgtggaagaatttgatctgcagtttatttttcagctgtgcagcattaccctgagcgcggaagtgatggcgtatattcataccatgaacccgagcgtgctggaagattggaactttggcctgagcccgccgccgaacggcaccctggaagatacctatcgctatgtgcagagccaggcgattacctgccagaaaccgaccccggaaaaagaaaaacaggatccgtataaagatatgagcttttgggaagtgaacctgaaagaaaaatttagcagcgaactggatcagtttccgctgggccgcaaatttctgctgcagagcggctatcgcggccgcaccagcgcgcgcaccggcattaaacgcccggcggtgagcaaaccgagcaccgcgccgaaacgcaaacgcaccaaaaccaaaaaa

SEQ ID NO: 3 - protein, 180 aa, homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 4 - DNA, 759 bp, artificial sequence
  cDNA
tctgcctccgcatcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacga

SEQ ID NO: 5 - DNA, 3371 bp, artificial sequence
  fusion protein coding sequence
atgggatggtcatgtatcatcctttttctagtagcaactgcaactggagtacattcacaggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctggaggcggaggatctggcggaggtggaagtggcggaggcggttctggcggtggcggatctgaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctcaggaggcggaggatctggcggaggtggaagtggcggaggcggttctggcggtggcggatctatggcccaggtgatcaataccaatagtctgtctctgctgacacagaacaacctgaacaaaagccagagcgccctgggcaccgccatcgagagactgagcagcggcctgcggatcaacagcgcaaaggatgatgccgctggacaggccatcgccaatagatttaccgccaacatcaagggcctgacccaggccagcagaaacgccaacgacggtatttctatcgcccagaccacggaaggcgccctgaacgagatcaacaacaatctgcagcgggttagagagctggccgtgcagagcgccaattcaacaaactcccagtccgacctggattctatccaggccgagatcacccagagactgaatgagatcgacagagtgtctggacaaacacaatttaacggcgtgaaggtgctggcccaggacaacaccctgaccattcaggtgggcgccaatgacggagaaaccatcgacatcgacctgaagcagatcaacagccagaccctgggcctggatagcctgaacgtgcagaaagcctacgacgtgaaagacaccgccgtcacaaccaaggcttatgccaacaacggcaccacactggacgtgagcggactggacgacgccgccatcaaggccgccacaggcggcacaaacggcacagcctctgtgacaggcggcgccgtgaagttcgacgctgataacaacaagtacttcgtgaccatcggcggcttcaccggcgccgacgccgctaagaacggcgactacgaggtgaacgtggccacagacggcaccgtgacactggctgctggcgccacaaagaccactatgcctgctggcgccaccaccaaaacagaggtgcaggagctgaaagatacccctgccgtggtgtccgcagacgccaaaaacgccctgatcgccggcggtgttgatgccaccgatgccaacggcgctgaactggtgaagatgtcttatacagataagaacggcaagacaatcgagggaggctacgccctcaaggccggagataagtactacgccgccgattacgacgaggccaccggagccatcaaggccaagaccaccagctacaccgccgccgatggcacaaccaagactgctgccaatcagctgggaggagtggacggcaaaaccgaagtcgtgaccatcgacggcaagacctacaacgcttctaaggccgccggccacgacttcaaggcccagcctgagctggccgaagctgctgccaagaccacagaaaacccactgcaaaagatcgatgccgccctggcccaggtggatgccctgagaagcgatctgggcgctgtgcaaaacagattcaacagcgccattaccaacctgggcaacacagtgaacaatctgagcgaggccagatctagaatcgaggacagcgactacgctacggaagtgtccaacatgagccgggcccaaatcctgcagcaggctggcactagcgtgctggcccaggccaaccaggtcccccagaacgtgctgagcctgctccgcccctggtgaggaggcggaggatctggcggaggtggaagtggcggaggcggttctggcggtggcggatctatggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaaggaggcggaggatctggcggaggtggaagtggcggaggcggttctggcggtggcggatctatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaaggaggcggaggatctggcggaggtggaagtggcggaggcggttctggcggtggcggatctcaccatcaccatcatcactga

SEQ ID NO: 6 - protein, 1072 aa, artificial sequence
  PROTEIN SEQUENCE CHIMERIC ANTIBODY PROTEIN
MGWSCIILFLVATATGVHSQAVVTQESALTTSPGETVTLTCRSSTGAVTISNYANWVQEKPDHLFTGLIGGTNNRAPGVPARFSGSLIGDKAALTITGAQTEDEAIYFCALWYNNQFIFGSGTKVTVLGGGGSGGGGSGGGGSGGGGSEVQLQQSGPVLVKPGASVKMSCKASGNTFTDSFMHWMKQSHGKSLEWIGIINPYNGGTSYNQKFKGKATLTVDKSSSTAYMELNSLTSEDSAVYYCARNGVRYYFDYWGQGTTLTVSSGGGGSAAAMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRPWGGGGSAAAMATPWSGYLDDVSAKFDTGVDNLQTQVTEALDKLAAKPSDPALLAAYQSKLSEYNLYRNAQSNTVKVFKDIDAAIIQNFRGGGGSAAAMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGGGSAAAHHHHHH

SEQ ID NO: 7 - DNA, 3371 bp, artificial sequence
  coding sequence for fusion protein
atgggatggtcatgtatcatcctttttctagtagcaactgcaactggagtacattcacaggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctggaggcggaggatctggcggaggtggaagtggcggaggcggttctggcggtggcggatctgaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctcaggaggcggaggatctggcggaggtggaagtggcggaggcggttctggcggtggcggatctatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaaggaggcggaggatctggcggaggtggaagtggcggaggcggttctggcggtggcggatctatggcccaggtgatcaataccaatagtctgtctctgctgacacagaacaacctgaacaaaagccagagcgccctgggcaccgccatcgagagactgagcagcggcctgcggatcaacagcgcaaaggatgatgccgctggacaggccatcgccaatagatttaccgccaacatcaagggcctgacccaggccagcagaaacgccaacgacggtatttctatcgcccagaccacggaaggcgccctgaacgagatcaacaacaatctgcagcgggttagagagctggccgtgcagagcgccaattcaacaaactcccagtccgacctggattctatccaggccgagatcacccagagactgaatgagatcgacagagtgtctggacaaacacaatttaacggcgtgaaggtgctggcccaggacaacaccctgaccattcaggtgggcgccaatgacggagaaaccatcgacatcgacctgaagcagatcaacagccagaccctgggcctggatagcctgaacgtgcagaaagcctacgacgtgaaagacaccgccgtcacaaccaaggcttatgccaacaacggcaccacactggacgtgagcggactggacgacgccgccatcaaggccgccacaggcggcacaaacggcacagcctctgtgacaggcggcgccgtgaagttcgacgctgataacaacaagtacttcgtgaccatcggcggcttcaccggcgccgacgccgctaagaacggcgactacgaggtgaacgtggccacagacggcaccgtgacactggctgctggcgccacaaagaccactatgcctgctggcgccaccaccaaaacagaggtgcaggagctgaaagatacccctgccgtggtgtccgcagacgccaaaaacgccctgatcgccggcggtgttgatgccaccgatgccaacggcgctgaactggtgaagatgtcttatacagataagaacggcaagacaatcgagggaggctacgccctcaaggccggagataagtactacgccgccgattacgacgaggccaccggagccatcaaggccaagaccaccagctacaccgccgccgatggcacaaccaagactgctgccaatcagctgggaggagtggacggcaaaaccgaagtcgtgaccatcgacggcaagacctacaacgcttctaaggccgccggccacgacttcaaggcccagcctgagctggccgaagctgctgccaagaccacagaaaacccactgcaaaagatcgatgccgccctggcccaggtggatgccctgagaagcgatctgggcgctgtgcaaaacagattcaacagcgccattaccaacctgggcaacacagtgaacaatctgagcgaggccagatctagaatcgaggacagcgactacgctacggaagtgtccaacatgagccgggcccaaatcctgcagcaggctggcactagcgtgctggcccaggccaaccaggtcccccagaacgtgctgagcctgctccgcccctggtgaggaggcggaggatctggcggaggtggaagtggcggaggcggttctggcggtggcggatctatggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaaggaggcggaggatctggcggaggtggaagtggcggaggcggttctggcggtggcggatctcaccatcaccatcatcactga

SEQ ID NO: 8 - protein, 1072 aa, artificial sequence
  fusion protein
MGWSCIILFLVATATGVHSQAVVTQESALTTSPGETVTLTCRSSTGAVTISNYANWVQEKPDHLFTGLIGGTNNRAPGVPARFSGSLIGDKAALTITGAQTEDEAIYFCALWYNNQFIFGSGTKVTVLGGGGSGGGGSGGGGSGGGGSEVQLQQSGPVLVKPGASVKMSCKASGNTFTDSFMHWMKQSHGKSLEWIGIINPYNGGTSYNQKFKGKATLTVDKSSSTAYMELNSLTSEDSAVYYCARNGVRYYFDYWGQGTTLTVSSGGGGSAAAMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGGGSAAAMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRPWGGGGSAAAMATPWSGYLDDVSAKFDTGVDNLQTQVTEALDKLAAKPSDPALLAAYQSKLSEYNLYRNAQSNTVKVFKDIDAAIIQNFRGGGGSAAAHHHHHH

SEQ ID NO: 9 - DNA, 1784 bp, artificial sequence
  fusion protein coding sequence
atgggatggtcatgtatcatcctttttctagtagcaactgcaactggagtacattcacaggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctggaggcggaggatctggcggaggtggaagtggcggaggcggttctggcggtggcggatctgaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctcaggaggcggaggatctggcggaggtggaagtggcggaggcggttctggcggtggcggatctatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaaggaggcggaggatctggcggaggtggaagtggcggaggcggttctggcggtggcggatctatggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaaggaggcggaggatctggcggaggtggaagtggcggaggcggttctggcggtggcggatctcaccatcaccatcatcactga

SEQ ID NO: 10 - protein, 556 aa, artificial sequence
  fusion protein
MGWSCIILFLVATATGVHSQAVVTQESALTTSPGETVTLTCRSSTGAVTISNYANWVQEKPDHLFTGLIGGTNNRAPGVPARFSGSLIGDKAALTITGAQTEDEAIYFCALWYNNQFIFGSGTKVTVLGGGGSGGGGSGGGGSGGGGSEVQLQQSGPVLVKPGASVKMSCKASGNTFTDSFMHWMKQSHGKSLEWIGIINPYNGGTSYNQKFKGKATLTVDKSSSTAYMELNSLTSEDSAVYYCARNGVRYYFDYWGQGTTLTVSSGGGGSAAAMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGGGSAAAMATPWSGYLDDVSAKFDTGVDNLQTQVTEALDKLAAKPSDPALLAAYQSKLSEYNLYRNAQSNTVKVFKDIDAAIIQNFRGGGGSAAAHHHHHH

SEQ ID NO: 11 - DNA, 1830 bp, artificial sequence
  coding sequence for fusion protein
atggcccaggtgatcaataccaatagtctgtctctgctgacacagaacaacctgaacaaaagccagagcgccctgggcaccgccatcgagagactgagcagcggcctgcggatcaacagcgcaaaggatgatgccgctggacaggccatcgccaatagatttaccgccaacatcaagggcctgacccaggccagcagaaacgccaacgacggtatttctatcgcccagaccacggaaggcgccctgaacgagatcaacaacaatctgcagcgggttagagagctggccgtgcagagcgccaattcaacaaactcccagtccgacctggattctatccaggccgagatcacccagagactgaatgagatcgacagagtgtctggacaaacacaatttaacggcgtgaaggtgctggcccaggacaacaccctgaccattcaggtgggcgccaatgacggagaaaccatcgacatcgacctgaagcagatcaacagccagaccctgggcctggatagcctgaacgtgcagaaagcctacgacgtgaaagacaccgccgtcacaaccaaggcttatgccaacaacggcaccacactggacgtgagcggactggacgacgccgccatcaaggccgccacaggcggcacaaacggcacagcctctgtgacaggcggcgccgtgaagttcgacgctgataacaacaagtacttcgtgaccatcggcggcttcaccggcgccgacgccgctaagaacggcgactacgaggtgaacgtggccacagacggcaccgtgacactggctgctggcgccacaaagaccactatgcctgctggcgccaccaccaaaacagaggtgcaggagctgaaagatacccctgccgtggtgtccgcagacgccaaaaacgccctgatcgccggcggtgttgatgccaccgatgccaacggcgctgaactggtgaagatgtcttatacagataagaacggcaagacaatcgagggaggctacgccctcaaggccggagataagtactacgccgccgattacgacgaggccaccggagccatcaaggccaagaccaccagctacaccgccgccgatggcacaaccaagactgctgccaatcagctgggaggagtggacggcaaaaccgaagtcgtgaccatcgacggcaagacctacaacgcttctaaggccgccggccacgacttcaaggcccagcctgagctggccgaagctgctgccaagaccacagaaaacccactgcaaaagatcgatgccgccctggcccaggtggatgccctgagaagcgatctgggcgctgtgcaaaacagattcaacagcgccattaccaacctgggcaacacagtgaacaatctgagcgaggccagatctagaatcgaggacagcgactacgctacggaagtgtccaacatgagccgggcccaaatcctgcagcaggctggcactagcgtgctggcccaggccaaccaggtcccccagaacgtgctgagcctgctccgcccctggtgaggaggcggaggatctggcggaggtggaagtggcggaggcggttctggcggtggcggatctatggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaa

SEQ ID NO: 12 - DNA, 609 bp, artificial sequence
  fusion protein coding sequence
atggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaaggaggcggaggatctggcggaggtggaagtggcggaggcggttctggcggtggcggatctatgtcgattgcaactattgtccctgagaatgccgttatagggcaggcggtcaatatcaggtctatggaaacggacattgtctcgctggatgaccggctactccaggctttttctggttcggcgattgccacggctgtggataaacagacgattaccaacaggattgaggaccctaatctggtgacggatcctaaagagctggctatttcgcaagagatgatttcagattataacctgtatgtttctatggtcagtacccttactcgtaaaggagtcggggctgttgaaacgctattacgctcatga

SEQ ID NO: 13 - DNA, 558 bp, artificial sequence
  sequence encoding fusion protein
atggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaaggaggcggaggatctggcggaggtggaagtggcggaggcggttctggcggtggcggatctatgccagacccggtgaataccccatggagcggttatctggatggcgtttccaaccaattcgaccaaggcgtgcaggatctgcacggcgccgtcgagcaggcgctgaaagatctttccgcgacgccatccgacccgaagctgctggcgaaataccagagcaagctgtccgagtacaacctgtaccgcaacgcgcaatccaacgcggtcaaggcgttcaaggacatcgacgccgccatcatccagaacttccgctaa

SEQ ID NO: 14 - protein, 8 aa, artificial sequence
  amino acid linking sequence
GGGGSAAA

SEQ ID NO: 15 - protein, 20 aa, artificial sequence
  amino acid linking sequence
GGGGSGGGGSGGGGSGGGGS

SEQ ID NO: 16 - protein, 44 aa, artificial sequence
  designed CTL epitope
MLPYLGWLVFAQHPNAELLKHYLFRNLPSYVYHQFIPNPLLGLD

SEQ ID NO: 17 - protein, 80 aa, Salmonella typhimurium strain LT2
MATPWSGYLDDVSAKFDTGVDNLQTQVTEALDKLAAKPSDPALLAAYQSKLSEYNLYRNAQSNTVKVFKDIDAAIIQNFR

SEQ ID NO: 18 - DNA, 243 bp, Salmonella typhimurium strain LT2
atggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaa

SEQ ID NO: 19 - protein, 101 aa, Salmonella typhimurium strain LT2
MSIATIVPENAVIGQAVNIRSMETDIVSLDDRLLQAFSGSAIATAVDKQTITNRIEDPNLVTDPKELAISQEMISDYNLYVSMVSTLTRKGVGAVETLLRS

SEQ ID NO: 20 - DNA, 306 bp, Salmonella typhimurium strain LT2
atgtcgattgcaactattgtccctgagaatgccgttatagggcaggcggtcaatatcaggtctatggaaacggacattgtctcgctggatgaccggctactccaggctttttctggttcggcgattgccacggctgtggataaacagacgattaccaacaggattgaggaccctaatctggtgacggatcctaaagagctggctatttcgcaagagatgatttcagattataacctgtatgtttctatggtcagtacccttactcgtaaaggagtcggggctgttgaaacgctattacgctcatga

SEQ ID NO: 21 - protein, 84 aa, Chromobacterium violaceum
MPDPVNTPWSGYLDGVSNQFDQGVQDLHGAVEQALKDLSATPSDPKLLAKYQSKLSEYNLYRNAQSNAVKAFKDIDAAIIQNFR

SEQ ID NO: 22 - DNA, 255 bp, Chromobacterium violaceum
atgccagacccggtgaataccccatggagcggttatctggatggcgtttccaaccaattcgaccaaggcgtgcaggatctgcacggcgccgtcgagcaggcgctgaaagatctttccgcgacgccatccgacccgaagctgctggcgaaataccagagcaagctgtccgagtacaacctgtaccgcaacgcgcaatccaacgcggtcaaggcgttcaaggacatcgacgccgccatcatccagaacttccgctaa

SEQ ID NO: 23 - protein, 100 aa, Chromobacterium violaceum
MNVIGPAASQALAQASDLGQTDAEMVSLEDRLIQAFSRSAVATDAERNDIMQRLERPELISNPAELFALQQRTANYNLEVSMISTLTRKTVGAVESLLRS

SEQ ID NO: 24 - DNA, 303 bp, Chromobacterium violaceum
atgaacgtcatcggccccgccgcatcgcaagcgctggcacaggccagcgatctaggacagacagacgccgaaatggtgtcgctggaagatagattgatccaggcattctcccgttcagccgtggccaccgacgccgaaagaaacgacatcatgcaacgactggagcggccggagttgatctccaatccagccgagctgttcgcgttgcagcagcgaaccgccaattacaacctcgaggtttcgatgatcagcacgctgacccgcaaaaccgtcggcgcggtggaaagcttgctgcgctcatga

SEQ ID NO: 25 - protein, 100 aa, Burkholderia thailandensis
MNITNPHAVSAQPSLSEIESSERPATLDAILKQTLADANDKSNVAKASIESRLADPVDFAHPEKLIALQTELSDYNIYVSLASTLARKAVSAVETLVKAQ

SEQ ID NO: 26 - DNA, 303 bp, Chromobacterium violaceum
atgaacatcacgaatccgcatgccgtgtccgcgcagccgtcgctcagcgaaatcgaatcgtccgagcggccggcgacgctcgacgcgatcctgaagcaaacgctcgccgatgcgaacgacaagtcgaatgtcgcgaaggcgagcatcgaatcgcggctcgccgatccggtcgacttcgcgcatccggagaagctgatcgcgctgcaaacggagctgtccgactacaacatctacgtctcgcttgcgagcacgctcgcgcgcaaggccgtctcggcggtggagacgctcgtcaaggcgcaatga

SEQ ID NO: 27 - protein, 97 aa, Shigella flexneri
MNYIYPVNQVDIIKASDFQSQEISSLEDVVSAKYSDIKMDTDIQVSQIMEMVSNPESLNPESLAKLQTTLSNYSIGVSLAGTLARKTVSAVETLLKS

SEQ ID NO: 28 - DNA, 294 bp, Shigella flexneri
atgaattacatttatccagtcaatcaggttgatattatcaaagccagtgattttcaatctcaagagatatcaagtctggaagacgtcgtgtcggctaaatatagtgatattaagatggatacagatattcaagtatcacaaataatggagatggtaagcaatccagaatcattaaacccagaatctttggccaagttacagacgacgctctcaaattattcaataggagtatcattagctggcacgttagcaagaaaaacagtttcggctgttgaaactttattaaagtcttaa

SEQ ID NO: 29 - protein, 71 aa, Candidatus Sodalis pierantonius
MDVSQLINTLSLLAHKAGNDVEDKMTADKLTDPASLLRAQFAVQQYSTFINYSSAILKTMKDMVGGIIAKI

SEQ ID NO: 30 - DNA, 216 bp, Candidatus Sodalis pierantonius
atggatgtgtcacaactcatcaatacgctttcccttctggcacataaggcgggaaatgatgtcgaagataaaatgacggcggataagcttactgaccctgcctccttactgcgagcgcagttcgcggtgcagcaatattcaacctttatcaactattccagcgcgatactcaagaccatgaaagatatggtcggcggcattatcgctaagatatga

SEQ ID NO: 31 - protein, 71 aa, Salmonella enterica subsp
MDIAQLVDMLSHMAHQAGQAINDKMNGNDLLNPESMIKAQFALQQYSTFINYESSLIKMIKDMLSGIIAKI

SEQ ID NO: 32 - DNA, 216 bp, Salmonella enterica subsp
atggatattgcacaattagtggatatgctctcccacatggcgcaccaggcaggccaggccattaatgacaaaatgaatggtaatgatttgctcaacccagaatcgatgattaaagcgcaatttgccttacagcagtattctacatttattaattacgaaagttcactgatcaaaatgatcaaggatatgcttagtggaatcattgctaaaatctga

SEQ ID NO: 33 - protein, 71 aa, Erwinia iniecta
MDIEAITSQLSQLVEQAGNEVQSKVTAADLNDPARMLQAQFAIQQYSVFVSYESAIMRAVKDMLSGIIQKI

SEQ ID NO: 34 - DNA, 216 bp, Erwinia iniecta
atggatatcgaagccatcaccagtcaattatcacaactggttgaacaagccggcaatgaggtgcaatcgaaagtcaccgccgccgatttgaacgaccccgcccgaatgctgcaagcacagtttgccattcaacagtattcggtattcgtcagctacgaaagcgccattatgcgggcggtgaaagacatgttgtcagggattattcagaagatatga

SEQ ID NO: 35 - protein, 71 aa, Cedecea neteri
MNVEQLVDSLSRLAHQAGQAIEDKMNGQDISNPEAMLKAQFAVQQYSTFINYESAMIKTIKDMLSGIITKI

SEQ ID NO: 36 - DNA, 216 bp, Cedecea neteri
atgaacgttgagcagcttgttgacagcttatcgcgcctggcacatcaggccgggcaggcgattgaggacaaaatgaacggccaggacatctcaaatccggaagccatgctcaaagcccagtttgccgtacagcagtactccacttttattaactacgagagtgcgatgatcaaaactatcaaagatatgctcagtggcattatcaccaaaatatga

SEQ ID NO: 37 - protein, 83 aa, Shigella flexneri
MSVTVPNDDWTLSSLSETFDDGTQTLQGELTLALDKLAKNPSNPQLLAEYQSKLSEYTLYRNAQSNTVKVIKDVDAAIIQNFR

SEQ ID NO: 38 - DNA, 252 bp, Shigella flexneri
atgagtgttacagtaccgaatgatgattggacattgagttcattatctgaaacttttgatgatggaactcaaacattacaaggtgaactaacattggcactagataaattagctaaaaatccttcgaatccacagttgctggctgaataccaaagtaaattatctgaatatacattatataggaacgcgcaatccaatacagtgaaagtgattaaggatgttgatgctgcaattattcaaaacttcagataa

SEQ ID NO: 39 - protein, 87 aa, Yersinia enterocolitica
MSNFSGFTKGNDIADLDAVAQTLKKPADDANKAVNDSIAALKDTPDNPALLADLQHSINKWSVIYNISSTIVRSMKDLMQGILQKFP

SEQ ID NO: 40 - DNA, 264 bp, Yersinia enterocolitica
atgagtaatttctctgggtttacaaaagggaacgatatcgctgacttagatgcggtggctcaaacgctcaagaagccagcagacgacgcaaacaaggcggttaatgactcgatagcagcattgaaagatacgcctgacaacccggcgttacttgctgacttacaacattcaattaataaatggtcggtaatttacaatataagctcaaccatagttcgtagcatgaaagacttaatgcaaggcatcctacagaagttcccataa

SEQ ID NO: 41 - protein, 73 aa, Escherichia coli
MNLSEITQQMGEVGKTLSDSVPELLNSTDLVNDPEKMLELQFAVQQYSAYVNVESGMLKTIKDLVSTISNRSF

SEQ ID NO: 42 - DNA, 222 bp, Escherichia coli
atgaatttatctgaaattactcaacaaatgggtgaagtaggtaaaacgctgagcgattctgtgccagagttacttaatagcaccgatttggttaatgaccctgaaaaaatgttagagttgcagtttgcggttcagcaatattctgcttatgttaacgtagaaagtggaatgttgaaaacgataaaagatctggtctcaaccatttctaaccgtagtttttaa

SEQ ID NO: 43 - protein, 85 aa, Pseudomonas aeruginosa
MAQIFNPNPGNTLDTVANALKEQANAANKDVNDAIKALQGTDNADNPALLAELQHKINKWSVIYNINSTVTRALRDLMQGILQKI

SEQ ID NO: 44 - DNA, 258 bp, Pseudomonas aeruginosa
atggcgcagatattcaaccccaacccggggaataccctcgataccgtggccaatgccctgaaggagcaggccaacgcagcgaacaaggacgtcaacgacgcgatcaaggccttgcaggggaccgacaatgccgataacccggcgctgctggccgagctgcaacacaagatcaacaagtggtcggtcatctacaacatcaactcgacggtgacccgtgcgctgcgcgacctgatgcaaggcatcctgcagaagatctga

SEQ ID NO: 45 - protein, 79 aa, Escherichia coli
MADWNGYIMDISKQFDQGVDDLNQQVEKALEDLATNPSDPKFLAEYQSALAEYTLYRNAQSNVVKAYKDLDSAIIQNFR

SEQ ID NO: 46 - DNA, 240 bp, Escherichia coli
atggcagattggaatggttatattatggatatcagcaaacaatttgatcagggcgttgatgatctgaaccagcaagttgaaaaagcgttggaggatttagcaaccaatccttccgacccgaaattccttgccgaatatcagagtgcattagctgagtatacattatatcgaaatgcgcaatctaacgttgtaaaagcgtataaagatcttgattctgcaatcatacaaaacttccgctaa

SEQ ID NO: 47 - protein, 115 aa, Yersinia enterocolitica
MPNIEIAQADEVIITTLEELGPVEPTTEQIMRFDAAMSEDTQGLGHSLLKEVSDIQKTFKTAKSDLHTKLAVSVDNPNDLMLMQWSLIRITIQEELIAKTAGRMSQNVETLSKGG

SEQ ID NO: 48 - DNA, 348 bp, Yersinia enterocolitica
atgccgaacatagaaatagctcaggccgatgaggtgatcataaccacgctggaggaattagggccggtagagccaacaactgagcaaataatgcgctttgatgcggcaatgtcagaagatacgcagggactgggccattcactcctcaaggaggttagtgatattcagaagacttttaagacggctaaaagtgacttgcacactaagctggctgtttcagttgataatcccaacgacctgatgctaatgcaatggtcacttatccgtataacaatccaagaagaacttatcgccaagacagccgggcgaatgagccaaaatgttgaaaccttgtcgaagggggggtga

SEQ ID NO: 49 - protein, 87 aa, Aeromonas hydrophila
MAIERFFDNGGDNNNTLDSVAKALKDQANESNQAVNKAIADMKTAPDNPALLAELQHKINKWSVVYNINSTVTRAMKDLMQGILQKI

SEQ ID NO: 50 - DNA, 264 bp, Aeromonas hydrophila
atggctatcgagagattttttgacaatggaggggataataacaacacccttgatagtgttgccaaagcattgaaagatcaggctaatgagagcaatcaagcagttaacaaagctattgctgatatgaaaactgccccggacaacccggccctgctggccgagctgcagcacaagatcaacaaatggtcggtggtgtacaacatcaactctaccgtgacgcgggcgatgaaagacttgatgcagggcattttacagaagatctga

SEQ ID NO: 51 - protein, 81 aa, Aeromonas hydrophila
MADIFNPGNTLDKVAENLKGQANTANDEVQKAIEALKTSPDDPARLAELQHKINKWSVIYNINSTVTRAIKDMMQSILQKI

SEQ ID NO: 52 - DNA, 246 bp, Aeromonas hydrophila
atggccgatatttttaacccgggaaataccctagacaaggtcgccgagaacctgaaggggcaggctaacacagccaatgatgaggttcaaaaagcgattgaagcgttgaaaacctccccggatgaccctgctcgtcttgccgagctgcaacacaagattaacaagtggtcagtgatctacaacattaattccaccgtgactcgagctattaaagacatgatgcaatctatcttgcagaagatttga

(19) United States
(12) Patent Application Publication
(10) Pub. No.: US 2024/0066118 A1
(43) Pub. Date: Feb. 29, 2024

(54) NEEDLE AND ROD PROTEINS AS INFLAMMASOME AGONISTS FOR AUGMENTING IMMUNE RESPONSES

(71) Applicant: Cornell University, Ithaca, NY (US)

(72) Inventor: Julie Magarian BLANDER, North Haven, CT (US)

(21) Appl. No.: 18/260,858

(22) PCT Filed: Jan. 12, 2022
(86) PCT No.: PCT/US2022/012167

§ 371 Date: Jul. 10, 2023

Related U.S. Application Data
(60) Provisional application No. 63/136,311, filed on Jan. 12, 2021.

(51) Int. Cl. A61K 39/39; A61K 39/00; A61P 35/00; A61P 37/04

(52) U.S. Cl. CPC A61K 39/39; A61K 39/001188; A61P 35/00; A61P 37/04; A61K 2039/53; A61K 2039/55516; A61K 2039/55555; A61K 2039/572; A61K 2039/6056; A61K 2039/6068

(57) ABSTRACT

Provided are compositions and methods that relate to Inflammasome Agonist Proteins (IAPS) that are used to stimulate immune responses. IAPS are used with Toll-Like Receptor (TLR) ligands, antigens, cell surface binding proteins, and binding partners that direct to IAPs or fusion proteins containing the IAP to a particular target. The IAP constructs can be used directly to stimulate immune responses, or in conjunction with other components such as antigens, whereby the IAPs function as adjuvants.

### CROSS-REFERENCE TO RELATED APPLICATION

This application claims priority to U.S. provisional patent application No. 63/136,311, filed Jan. 12, 2021, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Jan. 12, 2022, is named Cornell_Blander_ST25.txt, and is 72,725 bytes in size.

### FIELD

This disclosure generally relates to the field of enhancing immune responses and more specifically to needle and rod proteins and modifications thereof for use in enhancing immune responses.

### BACKGROUND OF THE DISCLOSURE

There are two types of adaptive immune responses - the cell-mediated immune response, which is carried out by activated T cells, and the humoral immune response, which is carried out via activated B cells and antibodies. The generated T cells and B cells are specific for the antigen, which may be in an invading pathogen. These cells can kill pathogens directly or secrete antibodies that enhance the phagocytosis of pathogens and disrupt their functioning and therefore control the infection. Adaptive immunity also includes a memory to provide the host with long-term protection from reinfection by the same pathogen.

It is now recognized that generation of adaptive immunity depends not only on exposure to antigen, but also the context in which the antigen is encountered. Adjuvants are substances that enhance an antigen specific immune response when used in conjunction with the antigen. The purpose of an adjuvant is to make the immune system take better note of an antigen. Recognition of antigens by antigen presenting cells (APCs) such as macrophages and dendritic cells essentially initiates a critical cascade of events ultimately leading to initiation of cell mediated and/or antibody mediated immune responses. Commonly used adjuvants for human vaccines include aluminum salts. Oil-based adjuvants are also being developed. As such, there is a continued need in the field of immunology to develop novel adjuvants.

### BRIEF SUMMARY OF THE DISCLOSURE

The present disclosure provides proteins, including but not limited to Inflammasome Agonist Proteins (IAP,) nucleic acids, Toll-Like Receptor (TLR) ligands, cancer antigens, certain binding partners, and combinations thereof, for promoting immune responses. Polynucleotides encoding the described proteins are also provided.

In various embodiments, the disclosure provides a method comprising introducing at least one IAP that is a Needle or Rod protein as further described below into an individual in need thereof to thereby stimulate an immune response in the individual. In non-limiting embodiments, the IAP is PrgI or CprI.

In embodiments, the IAP is a component of a fusion protein. In an alternative approach, the IAP (and/or other components as further described below) may be encoded by a polynucleotide that is introduced into cells of the individual such that the IAP (and other components if also encoded) is/are expressed within the cells. In a non-limiting embodiment, the polynucleotide is an mRNA that may be delivered with a suitable delivery reagent, including but not necessarily limited to liposomal nanoparticles.

In certain embodiments, a TLR agonist is also administered to the individual.

In certain embodiments, a cancer antigen is also administered to the individual.

In certain embodiments, a binding partner is also administered to the individual. In non-limiting examples, the binding partner binds with specificity to a dendritic cell surface marker, or binds with specificity to a cancer cell surface marker, or may be multivalent and binds to more than one target.

Representative IAPs, TLR agonists, cancer antigens, and binding partners, are described further below in the detailed description. In non-limiting examples, the IAP is one or a combination of PrgI, CprI, EprI, SSaG, or AscF.

In certain embodiments, the IAP is introduced into dendritic cells that activate T cells that participate in inhibiting growth of and/or killing cancer cells that express a cancer antigen. In non-limiting embodiments the cancer cells die by pyroptosis.

The disclosure includes all fusion proteins as described herein, and all polynucleotides encoding the fusion proteins. Expression vectors encoding the described fusion proteins are also provided, as are pharmaceutical formulations that comprise the IAPs, fusion proteins, and/or polynucleotides that encode the described components.

### BRIEF DESCRIPTION OF THE FIGURES

For a fuller understanding of the nature and objects of the disclosure, reference should be made to the following detailed description taken in conjunction with the accompanying figures.

FIG. 1. Experimental timeline. CD14⁺ monocytes were isolated from human peripheral blood. They were stimulated with human interleukin (IL)-4 and human granulocyte-monocyte colony stimulating factor (GM-CSF) for 4 days in complete RPMI media supplemented with 10% fetal bovine serum. At day 4, dendritic cells (DCs) were transduced with lentiviral vectors expressing the various needle and rod proteins. Then DCs either followed a 72 h incubation with the virus (A) or 20 hours with the virus (B). The end point is where cells and supernatant were recollected for Western blot, ELISA, and Flow cytometry measures.

FIG. 2. Phenotypic dendritic cell characterization by flow cytometry. Representative Flow cytometry plots at (A) isolation day, staining with CD14 and CD11b to check isolation purity, (B) Time course of expression of CD11c and HLA-DR, and (C) CD11c, HLA-DR, DC-SIGN and DEC-205, DC markers from days 4 and 5 as indicated.

FIG. 3. Expression levels of key inflammasome sensors and adaptors in human monocyte-derived DC. Levels of transcripts encoding for innate immune signaling proteins human Nod-like receptor family apoptosis inhibitory protein (NAIP), Toll-like receptor (TLR)5, Nod-like receptors NLRC4 and NLRP3, as measured by quantitative real-time polymerase chain reaction (qPCR) in 293T cells, as a negative control, THP-1 undifferentiated and differentiated with 100 nM PMA and primary human DC.

FIG. 4. Needle and Rod transduction into DC. Flow cytometry representative plots of human monocyte-derived DCs, double positive for DC markers (CD11c and DC-SIGN) and expressing GFP after transduction with lentiviral vectors expressing the Needle and Rod proteins (Prg-I and Prg-J, respectively) in combination with the green fluorescent protein (GFP) reporter gene. GFP is a marker of transduced cells.

FIG. 5. The Needle protein Prg-I activates the inflammasome in primary human dendritic cells. Dendritic cells (DC) were differentiated from CD14⁺ monocytes with GM-CSF and IL-4 and transduced with recombinant GFP lentiviruses encoding PrgI, PrgJ, or empty vector (GFP). (A) Representative Western blot inflammasome panel from concentrated supernatant or whole cell extract (WCE) of DC transduced with the recombinant lentiviruses as indicated. The blots were probed for the proteins shown on the left. CC3 is cleaved caspase-3. (B) The levels of IL-1β in the culture supernatants as measured by ELISA 72 hours post transduction.

FIG. 6. The Needle protein Prg-I activates inflammatory pyroptosis in primary human dendritic cells. (A) Representative Flow cytometry plots of Forward scatter (FSC) versus Side scatter (SSC) of DC without transduction or 72 hours post transduction with recombinant lentiviruses encoding PrgI (Needle) or PrgJ (Rod) proteins. Blue gates show live cells and red gates show dead cells. (B) Bar graphs showing the percentage of live cells in the cultures at 72 hours from the flow plots in (A).

FIG. 7. The Needle protein Prg-I activates inflammatory pyroptosis and IL-1β secretion by primary human dendritic cells. (A) Representative Flow cytometry plots of Forward scatter (FSC) versus Sytox-Red labeling of DC without transduction or 72 hours post transduction with recombinant lentiviruses encoding PrgI (Needle), or after stimulation with LPS for 24 hours, with ATP added in the last 30 minutes of the 24 hour incubation period to activate the inflammasome. Green gates show live cells and purple gates show dead cells. (B) Bar graphs showing the concentration of IL-1β in the DC culture supernatants measured by ELISA at the conclusion of each treatment as indicated in (A).

FIG. 8. The Needle proteins Prg-I and CprI activate the inflammasome in primary human dendritic cells. (A) Dendritic cells (DC) were differentiated from CD14⁺ monocytes with GM-CSF and IL-4 and transduced with recombinant GFP lentiviruses encoding PrgI, CprI or empty vector (GFP). Representative Western blot inflammasome panels on proteins from the concentrated supernatants or whole cell extract (WCE) are shown. The blots were immunoprobed for the proteins shown on the left of each panel. (B) Dendritic cells (DC) were differentiated from CD14⁺ monocytes with GM-CSF and IL-4 and transduced with recombinant GFP lentiviruses encoding PrgI, PrgJ, Cpr-I, CprJ, MxiI, BsaK, or were left untransduced. Representative Western blot inflammasome panels on proteins from the concentrated supernatants or whole cell extract (WCE) are shown. The blots were immunoprobed for the proteins shown on the left of each panel.

FIG. 9. Inflammasome agonist proteins EprI, SSaG, and AscF activate the inflammasome in primary human dendritic cells. Dendritic cells (DC) were differentiated from CD14⁺ monocytes with GM-CSF and IL-4 and transduced with recombinant GFP lentiviruses encoding EprI, SSaG, or were left untransduced. Some conditions as indicated also included stimulation of the DC with 100 ng/mL LPS. Representative Western blot inflammasome panels on proteins from the concentrated supernatants or whole cell extract (WCE) are shown. The blots were immunoprobed for the proteins shown on the left of each panel.

FIG. 10. The Needle protein Prg-I activates the inflammasome in a human macrophage cell line. The human monocytic cell line THP-1 was differentiated into macrophages with phorbol-myristate acetate (PMA). (A) Representative flow cytometry plot of the forward scatter (FSC) versus GFP expression (as a marker of transduction with the recombinant lentivirus) in PMA-differentiated THP1 cells transduced with Needle PrgI, Rod PrgJ, or Salmonella flagellin protein encoding lentiviral vectors. (B) Representative Western blot inflammasome panel on proteins from concentrated supernatant or whole cell extract (WCE) of PMA-differentiated THP-1 cells transduced with indicated lentiviral vectors expressing Prg-I, Prg-J, Salmonella flagellin or control GFP only (GFP). Blots were probed for the proteins shown on the left. (C) THP-1 cells subjected to CRISPR using control guide (gCtrl), or guides to Caspase-1 (gCasp1), to Caspase-4 (gCasp4), or to caspase-5 (gCasp5) were differentiated with PMA and transduced with PrgI-encoding lentiviral vector. The cleavage of Caspase-1 and pro-IL-1β in the concentrated supernatants is shown by Western blot. The concentrations of IL-1β were measured in the culture supernatant by ELISA. D) As in B, PMA-differentiated THP-1 cells were transduced with Needle PrgI, PrgI-flagellin fusion protein, Rod protein PrgJ, PrgJ-flagellin fusion protein encoding lentiviral vectors or control GFP only encoding lentiviral vector (GFP).

FIG. 11. Inflammasome agonist proteins PrgI, PrgJ, CprI, CprJ, MxiI, AscF, EprI, SSaG, and AscF elicit cell death in murine B16 melanoma cell line. (A) Morphology of B16-rtTA3-TRE3G-MxiI cells by light microscopy at 48 hours after the treatments shown: either no Dox and no selection antibiotics (Blasticidin and Puromycin), selection antibiotics without Dox, or with either 0.2 μg/mL (low) or 2 μg/mL (high) Dox. Representative micrographs shown in top row and numbers of viable cells shown at the bottom of each light micrograph. Cell Death phenotype at 48 hours of B16-rtTA3-TRE3G-MxiI cells as assessed by flow cytometry after staining with Annexin V-PE or 7AAD-PerCPCY5.5. The location of viable, early or late stage dying cells are indicated in the flow cytometry dot plot quadrants. Percentages of cells in each quadrant are indicated. (B) Cell Death phenotype at 48 hours of B16-rtTA3-TRE3G cells expressing either PrgI, PrgJ, CprI, CprJ, MxiI, AscF, EprI, SSaG, or AscF is shown. as assessed by flow cytometry after staining with Annexin V-PE or 7AAD-PerCPCY5.5. The location of viable, early or late stage dying cells are indicated in the flow cytometry dot plot quadrants. Percentages of cells in each quadrant are indicated. Bar graphs show on the Y-axis the percentage of cells that are either viable, in early Annexin-V⁺, early 7AAD⁺ or necrotic late Annexin-V⁺7AAD⁺ cells of total cells. (C) Western blot of B16-rtTA3-TRE3G cells in culture 48 hours post treatment with 2 μg/mL Dox. Whole cell extracts or concentrated supernatants were immunoprobed for Gasdermin D or the cleaved C-terminal form of Gasdermin D.

FIG. 12. Inflammasome agonist proteins PrgI, CprI and MxiI impair the growth of melanoma in mice. (A) Schematic of the experimental scheme and expectations of inflammasome agonist protein (IAP) expression in melanoma. rtTA3-TRE3G-Needle B16 tumor cells are induced to express Needle proteins PrgI, CprI and MxiI as examples by administration of Doxycycline (Dox) in the water. Resultant inflammasome activation in tumor cells, plasma membrane pore formation, and inflammatory tumor cell death leads to tumor regression and reversal of “cold” immune suppressive tumors into “hot” immuno-stimulatory tumors. (B) C57BL/6J mice were inoculated subcutaneously with 200,000 B16-rtTA3-TRE3G-PrgI or B16-rtTA3-TRE3G-CprI cells. At day 10, mice were placed on 2 mg/mL Dox water with 0.1 mg/mL sucrose or sucrose alone as a control (no Dox induction of IAP). Mice were taken off Dox or sucrose water on day 13. As a positive control for anti-tumor treatment, mice were injected with 300,000 gp100 melanoma antigen-specific PMEL T cell receptor transgenic CD8⁺ T cells that had been pre-activated with 1 ng/mL interleukin-2 (IL-2) for 24 hours prior to retroorbital sinus injection on day 12. Digital calipers were used to measure tumor length and width, and tumor volume was calculated using the equation (L×W²)/2. (C) C57BL/6J mice were inoculated subcutaneously with 200,000 B16-rtTA3-TRE3G-CprI or B16-rtTA3-TRE3G-MxiI cells. At day 10, B16-rtTA3-TRE3G-CprI or B16-rtTA3-TRE3G-MxiI tumor-bearing mice received an intra-tumoral injection of 4 mg/kg Dox at day 10 for 2 days. Digital calipers were used to measure tumor length and width, and tumor volume was calculated using the equation (L×W²)/2. Top right panels show representative pictures of B16-rtTA3-TRE3G-CprI tumor bearing mice before and after Dox injection on day 18. Tumor injection sites are indicated by the white arrows. Low right panels show representative subcutaneous tumor numbers in mice bearing B16-rtTA3-TRE3G-MxiI melanoma cells before or after Dox injection.

FIG. 13. Amino acid and nucleotide sequences for L1 major capsid protein from Human papilloma virus type 11 (HPV11). The sequence of the amino acid sequence is SEQ ID NO:1. A DNA sequence encoding SEQ ID NO:1 is provided as SEQ ID NO:2.

FIG. 14. Amino acid and nucleotide sequences of human cancer/testes antigen 1B (CTAG1B). The sequence of the amino acid sequence is SEQ ID NO:3. The sequence of a DNA sequence encoding CTAG1B is SEQ ID NO:4.

FIG. 15. Representative fusion protein constructs for delivering Needle and Rod protein adjuvants and antigen to human DEC205⁺ DC. Examples of chimeric fusion proteins targeting Inflammasome agonist proteins (IAP, Needle and Rod) and tumor associated antigen (TAA) to human DC with or without flagellin. The schematics of the chimeric proteins shown comprise antibody heavy and light chains with specificity to a surface receptor on human DC (DEC-205 in this case) (Single chain fragment variable of anti-human DEC-205) fused to IAP (either Needle or Rod) and a TAA (NY-ESO-1 in this case) and with or without flagellin. In FIG. 15, the nucleotide sequence of CHIMERIC ANTIBODY PROTEIN 1 is SEQ ID NO:5. The amino acid sequence of CHIMERIC ANTIBODY PROTEIN 1 is SEQ ID NO:6. The nucleotide sequence encoding CHIMERIC ANTIBODY PROTEIN 2 is SEQ ID NO:7. The amino acid sequence of CHIMERIC ANTIBODY PROTEIN 2 is SEQ ID NO:8. The nucleotide sequence encoding CHIMERIC ANTIBODY PROTEIN 3 is SEQ ID NO:9. The amino acid sequence of CHIMERIC ANTIBODY PROTEIN 3 is SEQ ID NO:10.

FIG. 16. Examples of nucleotide sequences encoding chimeric fusion proteins. (1) Flagellin and PrgI (Needle protein) separated by a linker region, (2) A Needle (PrgI) and a Rod (PrgJ) protein separated by a linker region, or (3) To Needle proteins PrgI and CrpI separated by a linker region. In FIG. 16, the nucleotide sequence encoding the chimeric fusion protein comprising flagellin and PrgI separated by a linker region is SEQ ID NO:11. The nucleotide sequence encoding the chimeric fusion protein comprising PrgI and PrgJ separated by a linker region is SEQ ID NO:12. The NUCLEOTIDE SEQUENCE encoding a chimeric fusion protein comprising PrgI and CprI separated by a linker region is SEQ ID NO:13.

FIG. 17. Chimeric antibody proteins delivering flagellin, a Needle protein and a tumor antigen to primary human dendritic cells elicit inflammasome activation. (A) Experimental schematic showing delivery of the chimeric antibody protein (biotherapeutic) to human dendritic cells (DC) by targeting the surface receptor DEC-205 for internalization. Flagellin in the chimeric antibody also elicits TLR5 activation, while flagellin and Needle protein elicit NLRC5 inflammasome activation. (B) Human DC were differentiated from CD14⁺ monocytes with GM-CSF and IL-4 and treated with 1 mg/mL of the chimeric proteins indicated in the presence or absence of 100 ng/mL LPS. Representative Western blot inflammasome panels on proteins from the concentrated supernatants or whole cell extract (WCE) at 24 hours post treatment are shown. The blots were immunoprobed for the proteins shown on the left of each panel. (C) ELISA for IL-1I3 and IL-6 at 24 hours on DC that had been treated with 1 or 10 μg/mL of chimeric antibody proteins as indicated. Transduction of DC with PrgI-encoding lentivirus served as a positive control.

FIG. 18. Chimeric antibody proteins delivering flagellin, a Needle protein and a tumor antigen to primary human dendritic cells elicit dendritic cell maturation and tumor antigen presentation to T cells. (A) Experimental scheme of human HLA-A2⁺ DC differentiation in culture and treatment with 1 μg/mL chimeric antibodies targeting DEC-205 and delivering Needle protein PrgI and tumor antigen NY-ESO-1 with and without flagellin, and coculture with Jurkat T cell line expressing an HLA-A2-restricted T cell receptor specific to the tumor antigen NY-ESO-1. (B) Flow cytometry on DC 6 hours post treatment with chimeric antibody proteins. Percentages of mature DC expressing both CD40 and CD86 are shown in the top right quadrants of each contour plot. Stimulation of DC with LPS or Escherichia coli bacteria served as a positive control and no treatment as a negative control. (C) ELISA for human IL-1β and IL-2 at 72 hours of DC-Jurkat NY-ESO-1 T cell coculture after treatment of DC with chimeric antibody proteins indicated on the X-axis.

### DETAILED DESCRIPTION

Although claimed subject matter will be described in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

Every numerical range given throughout this specification includes its upper and lower values, as well as every narrower numerical range that falls within it, as if such narrower numerical ranges were all expressly written herein. All ranges provided herein include all values that fall within the ranges to the tenth decimal place, unless indicated otherwise.

Any protein described herein can be administered in its protein form, or can be delivered to cells using a polynucleotide that encodes the protein, representative examples of which are discussed further below. Thus, where delivery of a protein is described, the disclosure includes delivery of the protein via a polynucleotide that encodes the described protein.

Throughout this application, the singular form encompasses the plural and vice versa. All sections of this application, including any supplementary sections or figures, are fully a part of this application.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

The term “therapeutically effective amount” as used herein refers to an amount of an agent sufficient to achieve, in a single or multiple doses, the intended purpose of treatment. The exact amount desired or required will vary depending on the particular compound or composition used, its mode of administration, patient specifics and the like. Appropriate effective amount can be determined by one of ordinary skill in the art informed by the instant disclosure using only routine experimentation.

All of the amino acid and nucleotide sequences associated with a GenBank or other database accession number are incorporated herein by reference as they exist on the filing date of this application or patent. The disclosure includes amino acid sequences that are from 80%-99% similar to those amino acid sequences, and includes amino acid sequences that include insertions and deletions. The disclosure also includes all polynucleotide sequences encoding the Needle and Rod family of proteins and fusion proteins, and all other proteins described herein, and all sequences complementary to those sequences. In certain embodiments, the disclosure relates to the L1 major capsid protein from Human papillomavirus type 11 (HPV 11). The amino acid sequence of this protein is available under UniProt reference P04012. A representative nucleotide sequence encoding this protein is available under NCBI reference no. NM_001327. In certain embodiments, the disclosure relates the NY-ESO-1 antigen. The amino acid sequence of the NY-ESO-1 antigen is available under UniProt reference P78358. A representative nucleotide sequence encoding the NY-ESO-1 antigen is available under NCBI reference NM_001327.

This disclosure provides compositions and methods for enhancing immune responses against antigens. The disclosure is based, in part, on the unexpected observations that members of the Needle and Rod protein family (herein referred to as Inflammasome Agonist Proteins or “IAPs”) can augment immune responses. IAPs are proteins that once delivered or expressed in target cells can activate the innate immune inflammasome and lead to the production of inflammatory mediators including inflammatory cytokines such as IL-1β or IL-18 and stimulatory damage-associated molecular patterns (DAMPs) such as high-mobility group box-1 (HMGB1) or purine metabolites (e.g. ATP, adenosine). Release of these factors as a result of inflammasome activation serves to augment the immune response by collectively inducing the production of other pro-inflammatory cytokines in myeloid cells (IL-1, TNF-α, IL-6, IL-8), and increasing endothelial cell expression of cell adhesion molecules such as ICAM-1 and VCAM-1. Compositions and methods are provided to use IAPs as adjuvants.

Needle and Rod proteins are structural components of the bacterial type III secretion system (T3SS) expressed by virulent bacteria such as Yersinia, Salmonella, Shigella flexneri, enteropathogenic Escherichia coli, and others. The bacterial T3SS enables bacteria to directly inject bacterial effector proteins into host cells across bacterial and host membranes. Needle proteins comprise the extracellular needle portion of the T3SS. Rod proteins comprise a central periplasmic Rod structure that connects the outer and inner rings of the T3SS apparatus. T3SS is closely related to the bacterial flagellar apparatus. While the flagellar apparatus secretes bacterial proteins into the environment outside the bacterium, the T3SS injects bacterial effectors into the host cell to manipulate host cell function. The host cell mounts an innate immune response to components of the T3SS such as Needle and Rod proteins.

In an aspect, this disclosure provides compositions comprising one or more IAPs, non-limiting embodiments of which include PrgI or CprI, with additional IAPs as discussed further below.

The IAPs can be used in various formulations, and may function as adjuvants, vaccines, and any other type of therapeutic formulation. Thus, in certain embodiments, an IAP and IAP-containing constructs described herein may have an adjuvant effect. For example, as demonstrated by way of the figures and examples of this disclosure, targeted delivery of an IAP to DCs, such as by using a DC-targeting binding partner, with co-delivery of an antigen, improves the DC response to the antigen. The improvement is relative to the DC response when provided with the antigen alone, e.g., in the absence of the IAP. The antigen may be a cancer antigen, or any other antigen to which an enhanced immune response is desired, such as antigens from pathogenic microorganisms, and viruses, non-limiting examples of which include SARS viruses and papilloma viruses. In other embodiments, use of IAP and IAP-containing constructs described herein elicit a direct IAP-induced effect, such as by delivering a described IAP or IAP-containing construct, but without a co-delivered antigen. In one embodiment, the direct IAP induced effect includes killing cancer cells into which said IAP or IAP containing construct is introduced.

The disclosure provides intra-tumoral delivery of IAPs to directly trigger tumor cell killing and/or to elicit inflammation within the tumor bed to recruit inflammatory cells, natural killer cells, or cytotoxic CD8⁺ T cells that eliminate tumor cells. The disclosure also provides fusion proteins comprising one or more IAPs. In addition to an IAP, the fusion protein may comprise other components, which may function as adjuvants (e.g., flagellins or fragments thereof, as further described below), and/or an antigen (e.g., a tumor associated antigen (TAA)), and/or an antibody (Ab) or an antigen binding fragment thereof, e.g., a binding partner that is specific for a particular antigen, such as a surface exposed antigen that is specific for a particular cell type, such as an antigen presenting cell, including but not limited to dendritic cells, and cancer antigens. Thus, in embodiments, a described IAP is provided as a component of fusion protein which further comprises, as described in more detail below, a Toll Like Receptor (TLR) agonist, a cancer antigen, a binding partner, or a combination thereof. In embodiments, a binding partner of this disclosure is a protein. In embodiments, the binding partner directs a fusion protein comprising the binding partner and an IAP to a target to which the binding partner binds with specificity. In embodiments, a binding partner may exclude a TLR ligand. In embodiments, a binding partner is an antibody or antigen binding fragment of the antibody.

Binding partners of this disclosure can be provided as intact immunoglobulins, or as fragments of immunoglobulins, including but not necessarily limited to antigen-binding (Fab) fragments, Fab′ fragments, (Fab′)₂ fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), single heavy chain only antibodies, isolated CDR regions, single-chain variable fragment (scFv), and other antibody fragments that retain antigen binding function. In embodiments, the binding partner is bi-specific or tri-specific. In embodiments, the binding partner binds with specificity to an epitope that is part of a DC surface marker or other antigen presenting cell. In embodiments, the DC surface marker is any of DEC-205, CLEC9, or 33D1. In embodiments, the binding partner may bind with specificity to any cancer cell surface marker and/or antigen, representative examples of which are discussed below in the context of fusion proteins that comprise the cancer antigens.

The fusion proteins can be used in vivo or ex vivo to augment immune response. For example, the fusion proteins can be used in the form of a vaccine formulation for mounting an immune response.

In embodiments, a fusion protein comprises a binding partner component, e.g., an antibody or antigen binding fragment thereof, as described above. The antibody may have specificity to an endocytic receptor on human DC or a receptor on tumor cells. For example, chimeric antibody fusion proteins of the present disclosure may be used ex vivo to deliver a TAA and adjuvant to DCs, which can then be administered to an individual to enhance immune response to the TAA. The chimeric antibody fusion proteins can be used in vivo for targeted delivery of an antigen and adjuvant to DCs in an individual. The chimeric antibody fusion proteins can also be used to target delivery of an IAP and/or other adjuvant (e.g., flagellin or fragment thereof) to tumor cells.

Also provided in this disclosure are recombinant viral vectors comprising nucleic acids encoding the IAP and/or flagellin. The recombinant viral vectors can be used in vivo to deliver the IAP and/or flagellin to tumor cells or other cells within the tumor microenvironment to elicit inflammation, recruitment of immune cells, and tumor killing. Immune cell recruitment would enable the activation of DCs that prime cytotoxic CD8⁺ T cells or activation of cytotoxic CD8⁺ T cells and/or reversal of their ‘exhausted’ phenotype. Cells that have been transformed or transfected with recombinant vectors encoding the IAP (Needle or Rod) or flagellin are also provided. Transfected cells may include prokaryotic cells, such as Gram-negative bacterial cells, eukaryotic cells, such as human DCs, tumor cells, and/or stromal cells within a tumor microenvironment or tumor bed.

Also provided in this disclosure are nucleic acids encoding the IAP and/or flagellin. These nucleic acids may be encapsulated in formulations that protect them from degradation and ensure intact delivery to cells through endocytosis or other means of internalization such as macropinocytosis or phagocytosis.

In an aspect, this disclosure provides methods for using the present compositions for generating or augmenting immune responses. The methods may involve in vivo or ex vivo use.

In an embodiment, the method comprises administering compositions comprising IAPs such as Needle and/or Rod proteins, alone or as part of a fusion protein to an individual in need of augmenting an immune response.

In an embodiment, the method comprises administering compositions comprising IAPs such as Needle and/or Rod proteins, as part of a chimeric antibody fusion protein that targets a receptor of choice on a cell type of choice in an individual in need of augmenting an immune response.

In an embodiment, the method comprises administering to an individual in need of treatment, a vector such as an adenoviral vector capable of infecting cells and comprising a sequence encoding the IAP adjuvant, such as a needle or rod protein, and optionally one or more of: an antigen, such as a tumor antigen, additional adjuvants, such as flagellin.

In an embodiment, the method comprises administering to an individual in need of treatment, a formulation that delivers messenger RNA molecules encoding for the IAP, such as needle, rod or flagellin protein or combinations thereof. Examples of delivery systems may be, but not limited to, (1) lipid-based such as lipid nanoparticles (LNPs) consisting of a cationic lipid, a helper lipid, cholesterol, and polyethylene glycol (PEG), which form stable particles that carry the mRNA in their inner core to protect it from degradation. (2) Another delivery system may be based on amino-polyesters formulated into LNPs to encapsulate the mRNA molecules. (3) Polymer based materials including poly(amido-amine), poly-beta amino-esters (PBAEs), and polyethylenimine (PEI). (4) Polyplexes consisting of PEGylated polyacridine peptides. (5) Self-assembling block polymers consisting of an outer PEG shell and polymeric nanoparticles used for mRNA delivery. (5) A combination of lipid-based and polymer-based hybrid mRNA delivery comprising an LNP to protect the mRNA and a polymer micelle to target endocytosis by cells.

In an embodiment, the method comprises exposing DCs ex vivo to fusion proteins comprising a Needle or Rod protein, an antigen, and an antibody or fragment thereof with specificity to a receptor on DC, and optionally with another adjuvant.

The adjuvant compositions of the present disclosure may comprise one or more Needle and/or Rod proteins. Members of the Needle and Rod protein family (referred to herein as Inflammasome agonist proteins—IAP) include, but are not limited to PrgI, PrgJ, CprI, CprJ, BsaK, BsaL, MxiI, MxiH, YscF, EscF, PscF, EprI, AscF, SsaG, and YscI. Examples of Needle proteins include PrgI, CprI, MxiH, YscF, EscF, PscF, EprI, SsaG and YscI. Examples of Rod proteins include PrgJ, CprJ, BsaK and MxiI. The Needle and Rod proteins described herein are members of the type three secretion system (T3SS) commonly found in pathogenic Gram-negative bacteria. The T3SS serves as a sensory probe to detect potential host organisms and secretes effector proteins to aid in establishing infection and avoiding the host's immune response. These Needle and Rod proteins are structural proteins that help form the needle-like injection apparatus of the T3 SS and anchor the apparatus to the inner and outer membranes of the bacteria. The Needle and Rod proteins can be from any pathogenic Gram-negative bacteria, including, but not limited to Escherichia coli, Salmonella typhimurium, Chromobacterium violaceum, Burkholderia thailandensis, Pseudomonas aeruginosa, Yersinia spp. and Shigella flexneri.

Some non-limiting examples of Needle and Rod proteins are provided below.

PrgI [Salmonella]. This is a needle protein that shares 100% identity with PrgI from other strains of Salmonella including but not limited to Salmonella enterica subsp. enterica serovars Ridge, Telelkebir and Kingabwa. The amino acid sequence of PrgI from Salmonella typhimurium strain LT2, ATCC 700720 is:

(SEQ ID NO: 17)
MATPWSGYLD DVSAKFDTGV DNLQTQVTEA LDKLAAKPSD
PALLAAYQSK LSEYNLYRNA QSNTVKVFKD IDAAIIQNFR
(NCBI Ref Seq:
NP_461794.1; NCBI Ref Seq: WP_000235228.1;
UNIPROT: P41784).

The nucleotide sequence encoding PrgI [Salmonella] from Salmonella typhimurium strain LT2 is:

(SEQ ID NO: 18)
ATGGCAACACCTTGGTCAGGCTATCTGGATGACGTCTCAG
CAAAATTTGATACGGGCGTTGATAATCTACAAACGCAGGT
AACAGAGGCGCTGGATAAATTAGCAGCAAAACCCTCCGAT
CCGGCGCTACTGGCGGCGTATCAGAGTAAGCTCTCGGAAT
ATAACTTGTACCGTAACGCGCAATCGAACACGGTAAAAGT
CTTTAAGGATATTGATGCTGCCATTATTCAGAACTTCCGT
TAA
(NCBI Gene ID: 1254396;
(European Nucleotide Archive
ENA coding: AAB60189.1).

PrgJ [Salmonella]. This is a rod protein. The amino acid sequence of PrgJ from Salmonella typhimurium strain LT2 and other strains of Salmonella is:

(NCBI Ref Seq: NP 461793.1; NCBI Ref Seq:
WP_000020431.1; UNIPROT: P41785)
(SEQ ID NO: 19)
MSIATIVPEN AVIGQAVNIR SMETDIVSLD
DRLLQAFSGS AIATAVDKQT ITNRIEDPNL
VTDPKELAIS QEMISDYNLY VSMVSTLTRK
GVGAVETLLR S

The nucleotide sequence encoding PrgJ [Salmonella] from Salmonella typhimurium strain LT2 is:

(SEQ ID NO: 20)
ATGTCGATTGCAACTATTGTCCCTGAGAATGCCGTTATAG
GGCAGGCGGTCAATATCAGGTCTATGGAAACGGACATTGT
CTCGCTGGATGACCGGCTACTCCAGGCTTTTTCTGGTTCG
GCGATTGCCACGGCTGTGGATAAACAGACGATTACCAACA
GGATTGAGGACCCTAATCTGGTGACGGATCCTAAAGAGCT
GGCTATTTCGCAAGAGATGATTTCAGATTATAACCTGTAT
GTTTCTATGGTCAGTACCCTTACTCGTAAAGGAGTCGGGG
CTGTTGAAACGCTATTACGCTCATGA.
(NCBI Gene ID: 1254395)

CprI. This is a needle protein also known as Cell Invasion protein-cytoplasmic. The amino acid sequence of CrpI from Chromobacterium violaceum ATCC 12472 is:

(SEQ ID NO: 21)
MPDPVNTPWS GYLDGVSNQF DQGVQDLHGA
VEQALKDLSAT PSDPKLLAKY QSKLSEYNLY
RNAQSNAVKA FKDIDAAIIQ NFR
(NCBI GenBank: AAQ60094.1)
(UNIPROT Q7NVC1).

The nucleotide sequence encoding CprI from Chromobacterium violaceum ATCC 12472 is:

(SEQ ID NO: 22)
ATGCCAGACCCGGTGAATACCCCATGGAGCGGTTATCTGG
ATGGCGTTTCCAACCAATTCGACCAAGGCGTGCAGGATCT
GCACGGCGCCGTCGAGCAGGCGCTGAAAGATCTTTCCGCG
ACGCCATCCGACCCGAAGCTGCTGGCGAAATACCAGAGCA
AGCTGTCCGAGTACAACCTGTACCGCAACGCGCAATCCAA
CGCGGTCAAGGCGTTCAAGGACATCGACGCCGCCATCATC
CAGAACTTCCGCTAA
(European Nucleotide Archive ENA
coding: AAQ60094.1).

CprJ. This is a rod protein. The amino acid sequence of CprJ from Chromobacterium violaceum ATCC 12472 is:

(SEQ ID NO: 23)
MNVIGPAASQ ALAQASDLGQ TDAEMVSLED RLIQAFSRSAV
ATDAERNDIM QRLERPELIS NPAELFALQQ RTANYNLEVS
MISTLTRKTV GAVESLLRS
(NCBI Ref Seq: WP_011135968.1).

The nucleotide sequence encoding CprJ from Chromobacterium violaceum ATCC 12472 is:

(SEQ ID NO: 24)
ATGAACGTCATCGGCCCCGCCGCATCGCAAGCGCTGGCACAGGCCAGCG
ATCTAGGACAGACAGACGCCGAAATGGTGTCGCTGGAAGATAGATTGAT
CCAGGCATTCTCCCGTTCAGCCGTGGCCACCGACGCCGAAAGAAACGAC
ATCATGCAACGACTGGAGCGGCCGGAGTTGATCTCCAATCCAGCCGAGC
TGTTCGCGTTGCAGCAGCGAACCGCCAATTACAACCTCGAGGTTTCGAT
GATCAGCACGCTGACCCGCAAAACCGTCGGCGCGGTGGAAAGCTTGCTG
CGCTCATGA
(NCBI Gene ID: 24949428).

BsaK. This is a rod protein. The amino acid sequence of BsaK from Burkholderia thailandensis E264, ATCC 700388 is:

(SEQ ID NO: 25)
MNITNPHAVS AQPSLSEIES SERPATLDAI LKQTLADAND
KSNVAKASIE SRLADPVDFA HPEKLIALQT ELSDYNIYVS
LASTLARKAV SAVETLVKAQ
(Burkholderia Genome DB Locus Tag: BTH_II0824;
NCBI Ref Seq: WP_009896108.1; UNIPROT: Q2T728).

The nucleotide sequence encoding BsaK from Chromobacterium violaceum ATCC 12472 is:

(SEQ ID NO: 26)
ATGAACATCACGAATCCGCATGCCGTGTCCGCGCAGCCGTCGCTCAGCG
AAATCGAATCGTCCGAGCGGCCGGCGACGCTCGACGCGATCCTGAAGCA
AACGCTCGCCGATGCGAACGACAAGTCGAATGTCGCGAAGGCGAGCATC
GAATCGCGGCTCGCCGATCCGGTCGACTTCGCGCATCCGGAGAAGCTGA
TCGCGCTGCAAACGGAGCTGTCCGACTACAACATCTACGTCTCGCTTGC
GAGCACGCTCGCGCGCAAGGCCGTCTCGGCGGTGGAGACGCTCGTCAAG
GCGCAATGA.
(Burkholderia Genome DB Locus Tag: BTH_II0824)

MxiI. This is a rod protein. The amino acid sequence of MxiI from Shigella flexneri 5a str. M90T is:

(SEQ ID NO: 27)
MNYIYPVNQV DIIKASDFQS QEISSLEDVV SAKYSDIKMD
TDIQVSQIME MVSNPESLNP ESLAKLQTTL SNYSIGVSLA
GTLARKTVSA VETLLKS )
(NCBI Ref Seq: NP_085301.1; UNIPROT: P0A225.

The nucleotide sequence encoding MxiI from Shigella flexneri 5a str. M90T is:

(SEQ ID NO: 28)
ATGAATTACATTTATCCAGTCAATCAGGTTGATATTATCAAAGCCAGTG
ATTTTCAATCTCAAGAGATATCAAGTCTGGAAGACGTCGTGTCGGCTAA
ATATAGTGATATTAAGATGGATACAGATATTCAAGTATCACAAATAATG
GAGATGGTAAGCAATCCAGAATCATTAAACCCAGAATCTTTGGCCAAGT
TACAGACGACGCTCTCAAATTATTCAATAGGAGTATCATTAGCTGGCAC
GTTAGCAAGAAAAACAGTTTCGGCTGTTGAAACTTTATTAAAGTCTTAA
(NCBI Nucleotide accession: AF386526).

SsaG. This is a needle protein expressed by Candidatus Sodalis pierantonius str SOPE, Sodalis praecaptivus and Sodalis sp. TME1. The amino acid sequence of SsaG from Candidatus Sodalis pieranton str. SOPE is:

(SEQ ID NO: 29)
MDVSQLINTL SLLAHKAGND VEDKMTADKL TDPASLLRAQ
FAVQQYSTFI NYSSAILKTM KDMVGGIIAK I
(NCBI Ref seq: WP_025245132; UNIPROT: W0HJ57).

The nucleotide sequence encoding SsaG from Candidatus Sodalis pierantonius str. SOPE is:

(SEQ ID NO: 30)
ATGGATGTGTCACAACTCATCAATACGCTTTCCCTTCTGGCACATAAGG
CGGGAAATGATGTCGAAGATAAAATGACGGCGGATAAGCTTACTGACCC
TGCCTCCTTACTGCGAGCGCAGTTCGCGGTGCAGCAATATTCAACCTTT
ATCAACTATTCCAGCGCGATACTCAAGACCATGAAAGATATGGTCGGCG
GCATTATCGCTAAGATATGA.
(European Nucleotide Archive ENA Coding:
AHF73809.1)

SsaG [Salmonella]. This is a needle protein expressed by but not limited to Salmonella enterica subsp. Enterica serovar Typhimurium str. LT2, Salmonella enterica subsp. Enterica serovar Typhimurium str. CT18, Salmonella enterica subsp. Enterica serovar Kottbus, Salmonella typhi, Chromobacterium violaceum ATCC 12472, DSM 30191/JCM 1249/NBRC 12614/NCIMB 9131/NCTC 9757. The amino acid sequence of SsaG from Salmonella is:

(SEQ ID NO: 31)
MDIAQLVDML SHMAHQAGQA INDKMNGNDL LNPESMIKAQ
FALQQYSTFI NYESSLIKMI KDMLSGIIAK I
(NCBI Ref Seq: WP_000350226.1; UNIPROT: 030903).

See also: SsaG from Salmonella enterica subsp. Enterica serovar Typhimurium str. LT2 NCBI Ref Seq NP_460371.1; SsaG from Salmonella enterica subsp. Enterica serovar Typhimurium str. CT18 NCBI Ref Seq NP_456122.1.

The nucleotide sequence encoding the SsaG [Salmonella] is:

(SEQ ID NO: 32)
ATGGATATTGCACAATTAGTGGATATGCTCTCCCACATGGCGCACCAGG
CAGGCCAGGCCATTAATGACAAAATGAATGGTAATGATTTGCTCAACCC
AGAATCGATGATTAAAGCGCAATTTGCCTTACAGCAGTATTCTACATTT
ATTAATTACGAAAGTTCACTGATCAAAATGATCAAGGATATGCTTAGTG
GAATCATTGCTAAAATCTGA
(European Nucleotide Archive ENA Coding:
CAD01959.1).

SsaG [Erwinia]. This is the Type III secretion system needle protein from Erwinia iniecta, Chromobacterium sp. ATCC 53434 and ATCC 12472, and Providencia rettgeri. It shares 90% protein homology with SsaG MULTIPSECIES SsaG above.

(SEQ ID NO: 33)
MDIEAITSQL SQLVEQAGNE VQSKVTAADL NDPARMLQAQ
FAIQQYSVFV SYESAIMRAV KDMLSGIIQK I
(UNIPROT: Q7NUW2; NCBI Ref Seq: WP_011136132.1).

The nucleotide sequence encoding SsaG [Erwinia] from Erwinia iniecta, Chromobacterium sp. ATCC 53434 and ATCC 12472, and Providencia rettgeri is:

(SEQ ID NO: 34)
ATGGATATCGAAGCCATCACCAGTCAATTATCACAACTGGTTGAACAAG
CCGGCAATGAGGTGCAATCGAAAGTCACCGCCGCCGATTTGAACGACCC
CGCCCGAATGCTGCAAGCACAGTTTGCCATTCAACAGTATTCGGTATTC
GTCAGCTACGAAAGCGCCATTATGCGGGCGGTGAAAGACATGTTGTCAG
GGATTATTCAGAAGATATGA
(NCBI Ref Seq: NC_005085.1).

SsaG [Cedecea neteri]. This is the Type III secretion system needle protein from Cedecea neteri. It has 100% identity with needle protein SsaG from Yokenella regensburgei including strain ATCC 43003.

(SEQ ID NO: 35)
MNVEQLVDSLSRLAHQAGQAIEDKMNGQDISNPEAMLKAQFAVQQYSTF
INYESAMIKTIKDMLSGIITKI
(UNIPROT: A0A089Q7Z7; NCBI GenBank:
AIR06619.1; NCBI Ref Seq WP_006820327.1).

The nucleotide sequence of SsaG [Cedecea neteri] is:

(SEQ ID NO: 36)
ATGAACGTTGAGCAGCTTGTTGACAGCTTATCGCGCCTGGCACATCAGG
CCGGGCAGGCGATTGAGGACAAAATGAACGGCCAGGACATCTCAAATCC
GGAAGCCATGCTCAAAGCCCAGTTTGCCGTACAGCAGTACTCCACTTTT
ATTAACTACGAGAGTGCGATGATCAAAACTATCAAAGATATGCTCAGTG
GCATTATCACCAAAATATGA
(European Nucleotide Archive ENA Coding:
AIR06619.1; NCBI Gene ID: 41070630).

MxiH. This is a needle protein. The amino acid sequence of MxiH from Shigella flexneri is:

(SEQ ID NO: 37)
MSVTVPNDDW TLSSLSETFD DGTQTLQGEL TLALDKLAKN
PSNPQLLAEY QSKLSEYTLY RNAQSNTVKV IKDVDAAIIQ
NFR
(UNIPROT P0A223; NCBI Ref Seq: NP_085300.1).

The nucleotide sequence encoding MxiH from Shigella flexneri is:

(SEQ ID NO: 38)
ATGAGTGTTACAGTACCGAATGATGATTGGACATTGAGTTCATTATCTGA
AACTTTTGATGATGGAACTCAAACATTACAAGGTGAACTAACATTGGCAC
TAGATAAATTAGCTAAAAATCCTTCGAATCCACAGTTGCTGGCTGAATAC
CAAAGTAAATTATCTGAATATACATTATATAGGAACGCGCAATCCAATAC
AGTGAAAGTGATTAAGGATGTTGATGCTGCAATTATTCAAAACTTCAGAT
AA
(European Nucleotide Archive ENA Coding:
AAA26530.1).

YscF. This is a needle protein uniquely expressed by Yersinia enterocolitica. The amino acid sequence of YscF from Yersinia enterocolitica is:

(SEQ ID NO: 39)
MSNFSGFTKG NDIADLDAVA QTLKKPADDA NKAVNDSIAA
LKDTPDNPAL LADLQHSINK WSVIYNISST IVRSMKDLMQ
GILQKFP
(NCBI Ref Seq: WP_010891228.1; UNIPROT: Q01247).

The nucleotide sequence encoding YscF from Yersinia enterocolitica is:

(SEQ ID NO: 40)
ATGAGTAATTTCTCTGGGTTTACAAAAGGGAACGATATCGCTGACTTAGA
TGCGGTGGCTCAAACGCTCAAGAAGCCAGCAGACGACGCAAACAAGGCGG
TTAATGACTCGATAGCAGCATTGAAAGATACGCCTGACAACCCGGCGTTA
CTTGCTGACTTACAACATTCAATTAATAAATGGTCGGTAATTTACAATAT
AAGCTCAACCATAGTTCGTAGCATGAAAGACTTAATGCAAGGCATCCTAC
AGAAGTTCCCATAA
(European Nucleotide Archive ENA Coding:
AAC37023.1).

EscF. This is a needle protein. Different strains of Escherichia coli express this protein at 100% homology and include but not limited to Escherichia coli strains O157:H7; O145:H28; O145; O145:H25; O111:H−; O8O:H26; O103; O11; O157; O26; O145:H28 str. RM12581; O127:H6 (strain E2348/69/EPEC); O157:H7 (strain EC869); O69:H11 str. 08-4661; O26:H11; DEC2C; O121:H19; O145:NM; Xuzhou21; O118:H16 str. 2009C-4446; 4.0967), as well as Shigella boydii and Escherichia albertii. The amino acid sequence of EscF from Escherichia coli is:

(SEQ ID NO: 41)
MNLSEITQQM GEVGKTLSDS VPELLNSTDL VNDPEKMLEL
QFAVQQYSAY VNVESGMLKT IKDLVSTISN RSF
(NCBI Ref Seq: AAG58816; UNIPROT: O05282).

The nucleotide sequence encoding EscF from Escherichia coli is:

(SEQ ID NO: 42)
ATGAATTTATCTGAAATTACTCAACAAATGGGTGAAGTAGGTAAAACGCT
GAGCGATTCTGTGCCAGAGTTACTTAATAGCACCGATTTGGTTAATGACC
CTGAAAAAATGTTAGAGTTGCAGTTTGCGGTTCAGCAATATTCTGCTTAT
GTTAACGTAGAAAGTGGAATGTTGAAAACGATAAAAGATCTGGTCTCAAC
CATTTCTAACCGTAGTTTTTAA
(European Nucleotide Archive ENA Coding:
AAC31497.1).

PscF. This is a major component of the T3SS needle structure. The amino acid sequence of PscF from Pseudomonas aeruginosa is:

(SEQ ID NO: 43)
MAQIFNPNPG NTLDTVANAL KEQANAANKD VNDAIKALQG
TDNADNPALL AELQHKINKW SVIYNINSTV TRALRDLMQG ILQKI
(NCBI Ref Seq: NP 250410.1; UNIPROT: P95434).

The nucleotide sequence encoding PscF from Pseudomonas aeruginosa is:

(SEQ ID NO: 44)
ATGGCGCAGATATTCAACCCCAACCCGGGGAATACCCTCGATACCGTGGC
CAATGCCCTGAAGGAGCAGGCCAACGCAGCGAACAAGGACGTCAACGACG
CGATCAAGGCCTTGCAGGGGACCGACAATGCCGATAACCCGGCGCTGCTG
GCCGAGCTGCAACACAAGATCAACAAGTGGTCGGTCATCTACAACATCAA
CTCGACGGTGACCCGTGCGCTGCGCGACCTGATGCAAGGCATCCTGCAGA
AGATCTGA
(European Nucleotide Archive ENA Coding:
AAC44777.1).

EprI. This is a T3SS needle complex protein. The amino acid sequence of EprI from Escherichia coli is:

(SEQ ID NO: 45)
MADWNGYIMD ISKQFDQGVD DLNQQVEKAL EDLATNPSDP
KFLAEYQSAL AEYTLYRNAQ SNVVKAYKDL DSAIIQNFR
(NCBI Ref Seq: WP_000796272.1; UniProt:
B2XU12).

The nucleotide sequence encoding EprI from Escherichia coli is:

(SEQ ID NO: 46)
ATGGCAGATTGGAATGGTTATATTATGGATATCAGCAAACAATTTGATCA
GGGCGTTGATGATCTGAACCAGCAAGTTGAAAAAGCGTTGGAGGATTTAG
CAACCAATCCTTCCGACCCGAAATTCCTTGCCGAATATCAGAGTGCATTA
GCTGAGTATACATTATATCGAAATGCGCAATCTAACGTTGTAAAAGCGTA
TAAAGATCTTGATTCTGCAATCATACAAAACTTCCGCTAA
(European Nucleotide Archive ENA Coding:
ACD01058.1).

YscI. This is a basal body protein from Yersinia enterocolitica. The amino acid sequence of YscF from Yersinia enterocolitica is:

(SEQ ID NO: 47)
MPNIEIAQAD EVIITTLEEL GPVEPTTEQI MRFDAAMSED
TQGLGHSLLK EVSDIQKTFK TAKSDLHTKL AVSVDNPNDL
MLMQWSLIRI TIQEELIAKT AGRMSQNVET LSKGG
(NCBI Ref Seq: WP_010891231.1; UniProt:
Q01250).

Shares 100% identity with UniProt: Q7BRZ3).

The nucleotide sequence encoding YscI from Yersinia enterocolitica is:

(SEQ ID NO: 48)
ATGCCGAACATAGAAATAGCTCAGGCCGATGAGGTGATCATAACCACGCT
GGAGGAATTAGGGCCGGTAGAGCCAACAACTGAGCAAATAATGCGCTTTG
ATGCGGCAATGTCAGAAGATACGCAGGGACTGGGCCATTCACTCCTCAAG
GAGGTTAGTGATATTCAGAAGACTTTTAAGACGGCTAAAAGTGACTTGCA
CACTAAGCTGGCTGTTTCAGTTGATAATCCCAACGACCTGATGCTAATGC
AATGGTCACTTATCCGTATAACAATCCAAGAAGAACTTATCGCCAAGACA
GCCGGGCGAATGAGCCAAAATGTTGAAACCTTGTCGAAGGGGGGGTGA
(European Nucleotide Archive ENA Coding:
AAC37026.1).

AscF. This is a needle complex major subunit protein from Aeromonas hydrophila. It is also expressed by Aeromonas sp. ASNIH4. The amino acid sequence of AscF Needle-like protein from Aeromonas hydrophila is:

(SEQ ID NO: 49)
MAIERFFDNG GDNNNTLDSV AKALKDQANE SNQAVNKAIA
DMKTAPDNPA LLAELQHKIN KWSVVYNINS TVTRAMKDLM
QGILQKI
(UniProt: Q1EHA3).

See also Aeromonas sp. ASNIH4 AscF type III secretion system needle major subunit protein (UniPrto: Q1EHA3).

The nucleotide sequence encoding AscF from Aeromonas hydrophila is:

(SEQ ID NO: 50)
ATGGCTATCGAGAGATTTTTTGACAATGGAGGGGATAATAACAACACCCT
TGATAGTGTTGCCAAAGCATTGAAAGATCAGGCTAATGAGAGCAATCAAG
CAGTTAACAAAGCTATTGCTGATATGAAAACTGCCCCGGACAACCCGGCC
CTGCTGGCCGAGCTGCAGCACAAGATCAACAAATGGTCGGTGGTGTACAA
CATCAACTCTACCGTGACGCGGGCGATGAAAGACTTGATGCAGGGCATTT
TACAGAAGATCTGA
(European Nucleotide Archive ENA Coding:
ABF70179.1).

AscF Needle-like protein. This is a T3SS needle-like protein from Aeromonas hydrophila. It has 90% homology to Type III export protein pscF from Aeromonas allosaccharophila and other Aeromonas sp. The amino acid sequence of AscF Needle-like protein from Aeromonas hydrophila is:

(SEQ ID NO: 51)
MADIFNPGNT LDKVAENLKG QANTANDEVQ KAIEALKTSP
DDPARLAELQ HKINKWSVIY NINSTVTRAI KDMMQSILQK I
(UniProt: Q699P8).

The nucleotide sequence encoding AscF Needle-like from Aeromonas hydrophila is:

(SEQ ID NO: 52)
ATGGCCGATATTTTTAACCCGGGAAATACCCTAGACAAGGTCGCCGAGAA
CCTGAAGGGGCAGGCTAACACAGCCAATGATGAGGTTCAAAAAGCGATTG
AAGCGTTGAAAACCTCCCCGGATGACCCTGCTCGTCTTGCCGAGCTGCAA
CACAAGATTAACAAGTGGTCAGTGATCTACAACATTAATTCCACCGTGAC
TCGAGCTATTAAAGACATGATGCAATCTATCTTGCAGAAGATTTGA
(European Nucleotide Archive ENA Coding:
AAS91831.1).

While sequences or reference numbers have been provided for needle and rod proteins from specific bacteria or strains, it will be appreciated that these proteins from other strains or bacteria may also be used. Needle and Rod proteins from other sources such as other bacteria, and nucleotides sequences encoding such proteins, that have at least 80%, at least 85%, at least 90%, at least 95%, at least 98%, at least 99%, or 100% identity with the sequences/references provided herein may be used.

In this disclosure, it was surprisingly observed that Needle and Rod proteins activate inflammasomes. Based at least in part on these observations, this disclosure provides compositions and methods for enhancing an immune response against an antigen. The inflammasomes are a group of multimeric protein complexes containing an inflammasome sensor molecule, an adaptor protein and caspase 1. Inflammasome formation is triggered during infections. Once the protein complexes have formed, the inflammasomes activate caspase 1, which proteolytically activates the pro-inflammatory cytokines interleukin-1β (IL-1β) and IL-18. Based on the present disclosure, it is considered that the Needle and Rod proteins can serve as a trigger for inflammasome assembly and activation. These proteins may bind to the sensor protein NAIP found in the cytosol of human cells. NAIP, in turn, binds to and activates the nucleotide-binding domain, leucine rich containing (NLR) protein NLRC4. Activated NLRC4 recruits the adapter protein ASC which in turn recruits and activates caspase-1 enzyme to form the assembled NLRC4 inflammasome. The complete NLRC4 inflammasome is a multi-protein enzymatic complex that mediates the activation of the proinflammatory cytokines IL-1β and IL-18. Production of IL-1β and IL-18 enhances antigen-specific antibody production and enhances CD4⁺ and CD8⁺ T cell activity. As such, activation of inflammasomes may be evaluated by one or more of the following criteria: activation and cleavage of caspase 1, cleavage of pro-IL-1β and pro-IL-18 into their biologically active forms of IL-1β, and/or IL-18, cleavage of Gasdermin D and in some cases Gasdermin E, or another step in the inflammasome activation pathway.

A needle or rod protein can be used in in conjunction with another adjuvant in adjuvant compositions, or in a fusion protein. For example, a Needle and rod protein may be used in conjunction with activators of the Toll-like receptor (TLR) pathway, such as a TLR ligand. Thus, in embodiments, compositions of this disclosure that are administered to an individual in need thereof may comprise TLR ligands, which may be TLR agonists. In embodiments, the TLR ligands are selected from TLR1, TLR2, TLR3, TLR4, TLR5, TLR6, TLR7, TLR8, TLR9, TLR10, TLR11, TLR12, and TLR13 ligands. In embodiments, the TLR ligand is a TLR3 ligand. In one embodiment, a TLR3 ligand is a molecule or complex that comprises a pathogen-associated molecular pattern (PAMP). In an embodiment, the TLR3 ligand comprises a single stranded or double stranded RNA. In embodiments, the TLR3 ligand is polyinosinic:polycytidylic acid, also referred to in the art as poly I:C, poly(I:C) and PIC, or rintatolimod (sold under the tradename AMPLIGEN). In embodiments, the TLR3 ligand is high molecular weight poly I:C or low molecular weight poly I:C. In embodiments, the TLR ligand is a TLR9 ligand. In an embodiment, the TLR ligand comprises unmethylated cytosine-phosphate-guanine (CpG) dinucleotides. In an embodiment, the TLR ligand comprises a TLR4 ligand, one example of which comprises a lipopolysaccharide (LPS). In an embodiment, a composition of this disclosure is administered with monophosphoryl lipid A (MPLA).

Another non-limiting example of a TLR activator is flagellin, or a fragment thereof, that binds to TLR. Flagellin is a protein expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by Toll-like receptor 5 (TLR5) as well as by Nod-like receptors (NLRB) Ipaf and Naip5 (Franchi et al (2006) Nat Immunol 7(6):576-582; Miao et al (2006) Nat Immunol 7(6):569-575; and Ren et al (2006) PLoS Pathog 2(3):e18). Flagellin sequences are well known in the art (for example, see U.S. Pat. No. 9,314,484, incorporated herein by reference). Flagellin sequences from different bacteria are available. For example, GenBank accession numbers D13689 (nucleic acid sequence), YP_275549, YP_275550, AAU18718, AAU18717, ZP_00743095, EAO52626, YP_315348, AAT28337, AAT28336, AAT28335, AAT28334, AAT28333, AAZ36356, AAZ33167, AAZ94424, AAZ91670, BAD18052, and BAD18051. Flagellin activates both Toll-like receptor 5 and the NLRC4 inflammasome. According to present knowledge, Rod and Needle proteins activate the NLR inflammasome pathway.

For the present compositions and methods, flagellin or a fragment thereof may be used. A fragment of flagellin may be a TLR ligand or an NLR ligand, or both. An example of a flagellin fragment that is only a TLR ligand is a flagellin that is missing the 20 C-terminal amino acids. An example of a Flagellin fragment that is a NLR ligand is the 20 C-terminal amino acids of flagellin (See U.S. Pat. No. 9,314,484)

The term “flagellin” as used herein encompasses any polypeptide that binds a naturally occurring TLR5 and triggers at least one of the biological functions of the TLR5 in antigen-presenting cells upon such binding. Thus, a flagellin may be a polypeptide comprising any of the naturally occurring bacterial flagellin proteins. A flagellin may also be a polypeptide that is substantially identical with any of the naturally occurring bacterial flagellin proteins at the amino acid sequence level, wherein the polypeptide is capable of binding a naturally occurring TLR5. Furthermore, a flagellin may be a polypeptide that is substantially identical with the 170 residues from the N terminus and 90 residues from the C terminus of any of the naturally occurring bacterial flagellin proteins at the amino acid sequence level, wherein the polypeptide is capable of binding a naturally occurring TLR5. The flagellin encompassed by this disclosure may also comprise a modification, such as glycosylation or phosphorylation. The flagellin may also be a mutant or protein variant of flagellin. Flagellin sequences are disclosed in U.S. Pat. No. 9,314,484, incorporated herein by reference. Flagellin represents both a TLR and an NLR ligand.

In an aspect, this disclosure provides fusion proteins. A fusion protein typically comprises all or a biologically active part of a first peptide or polypeptide operably linked to all or a biologically active part of a second peptide or polypeptide. A third peptide or polypeptide may be operatively linked to the second polypeptide and so on. The term “operably linked” means that a first polypeptide and the additional polypeptide(s) are produced in the same reading frame. The fusion protein can be produced using standard molecular biology techniques, when given the benefit of the present disclosure. In embodiments, the fusion protein is produced by expression from an expression vector that encodes the first, second, third etc. peptides or polypeptides, wherein the first, second, third etc. peptides or polypeptides are encoded in the same open reading frame. Such expression vectors are encompassed by the disclosure, and a wide variety of systems for expressing and separating fusion polypeptides are commercially available and can be adapted for producing fusion proteins of the present disclosure. The first, second, third etc. peptides or polypeptides may be contiguous without intervening amino acids (e.g., linkers), or amino acids may be present between the different peptide or polypeptide sequences. Additional amino acids may be present at the N or C terminus of the fusion protein (e.g., a polyhistidine tag or other tags to aid with purification, stability etc.).

Amino acid linkers may be mainly composed of relatively small, neutral amino acids, such as glycine, serine, and alanine, and can include multiple copies of a sequence enriched in glycine and serine. The linker may comprise from 1-100 amino acids, inclusive, and including all numbers and ranges of numbers there between. In specific and non-limiting embodiments, the linker comprises 3, 4, 5, 6, 7, 8, 9, 10, 11, 12, 13, 14, 15, 16, 17, 18, 19, and 20 amino acids. The peptides and polypeptides of this disclosure may be configured in a variety of N-terminus- to -C terminus orientations. In an example, the linker may be the glycine-serine-alanine linker G₄SA₃ with the following amino acid sequence: GGGGSAAA (SEQ ID NO:14). Another example of a linker is a glycine-serine linker (G₄S)₄ with the following amino acid sequence: GGGGSGGGGSGGGGSGGGGS (SEQ ID NO:15). The fusion proteins may also contain a polyhistidine tag (e.g., a hexahistidine tag).

The fusion proteins of the present disclosure may comprise an antigen. The antigen can be any antigen, such as an antigen from a mammalian cell or a microorganism. The microorganism may be a pathogenic microbe. For example, the antigen may be from bacteria, virus, fungus, parasite etc. An antigen may be from a mammalian cell, such as a human cell. For example, an antigen may be from tumor cells. Many tumor antigens are known in the art. Any protein antigen expressed by a tumor cell is contemplated for use in the present invention. Examples of tumor antigens are those which are known to be highly immunogenic (e.g., that comprise immunodominant epitopes that will stimulate a strong anti-tumor immune response). Non-limiting examples of tumor antigen that may be used in the fusion proteins of the present invention include ErbB receptors, Melan A [MART1], gp100, tyrosinase, TRP-1/gp 75, and TRP-2 (in melanoma; for additional examples, see also a list of antigens provided in Storkus and Zarour, Forum (Genova), 2000 July-September, 10(3):256-270); MAGE-1 and MAGE-3 (in bladder, head and neck, and non-small cell carcinoma); HPV E6 and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers), PIA tumor antigen (e.g., CTL epitope LPYLGWLVF) as disclosed in WO 98/56919), and such shared tumor-specific antigens as MAGE-2, MAGE-4, MAGE-6, MAGE-10, MAGE-12, BAGE-1, CAGE-1,2,8, CAGE-3 to 7, LAGE-1, NY-ESO-1/LAGE-2, NA-88, GnTV, and TRP2-INT2 a chimeric tumor CTL epitope string such as MLPYLGWLVF-AQHPNAELL-KHYLFRNL-SPSYVYHQF-IPNPLLGLD (SEQ ID NO:16). (see, e.g., PCT Application No. WO 98/56919). (Robson et al., Curr Opin Immunol. 2010 January)28).

A fusion protein may comprise or consist essentially of a Needle and/or Rod protein and flagellin, with or without intervening linker amino acids. Needle or rod proteins for a fusion protein comprising the protein and flagellin include proteins PrgI, PrgJ, CprI, CprJ, BsaK, BsaL, MxiI, MxiH, YscF, EscF, PscF, EprI, AscF, SsaG, and YscI. Examples of fusion proteins comprising or consisting essentially of a rod or needle protein and flagellin include a fusion protein comprising or consisting essentially of: PrgI and flagellin, PrgJ and flagellin, CprI and flagellin, CprJ and flagellin, BsaK and flagellin, MxiI and flagellin, MxiH and flagellin, YscF and flagellin, EscF and flagellin, PscF and flagellin, EprI and flagellin, and YscI and flagellin. In embodiments, a fusion protein may comprise more than one needle or rod protein.

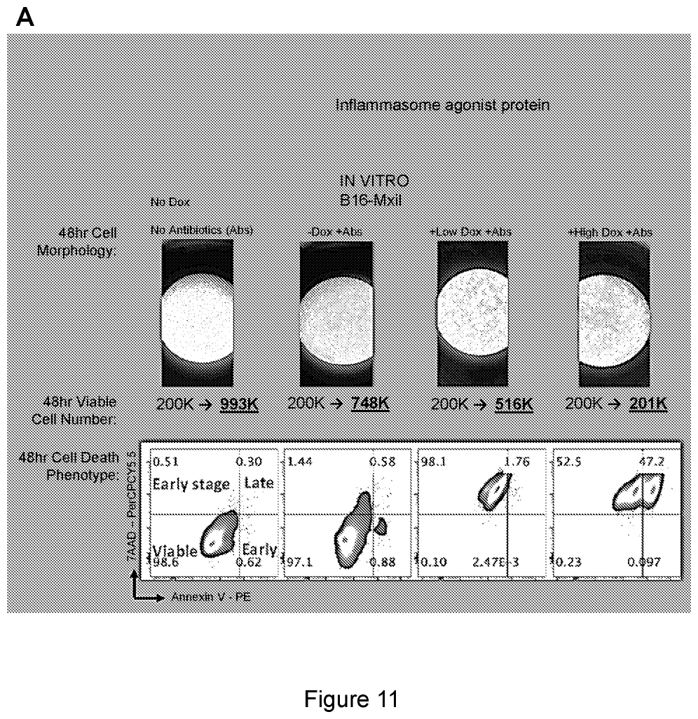

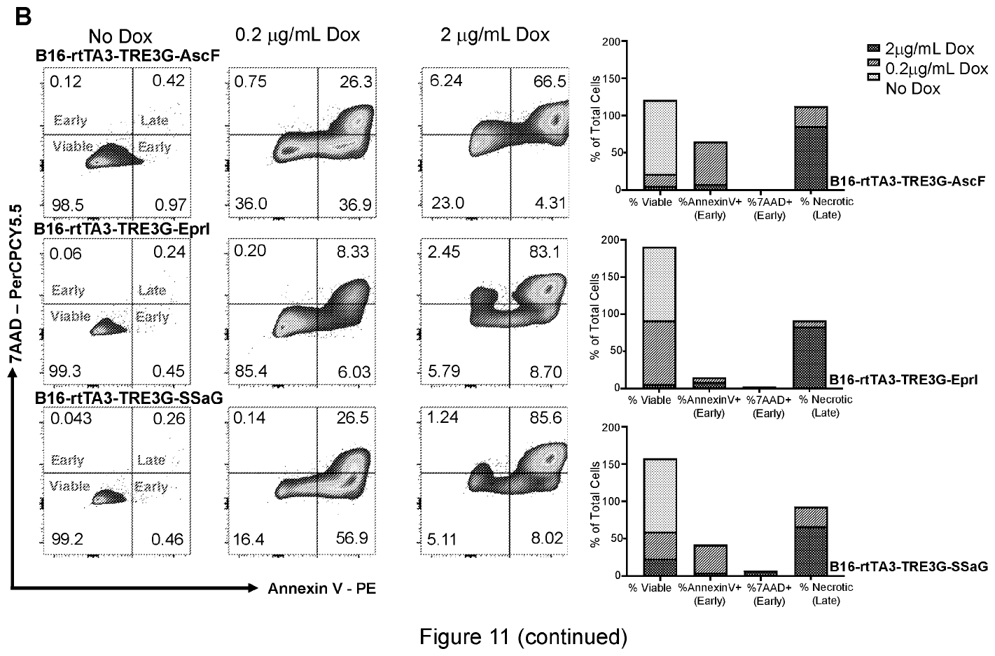

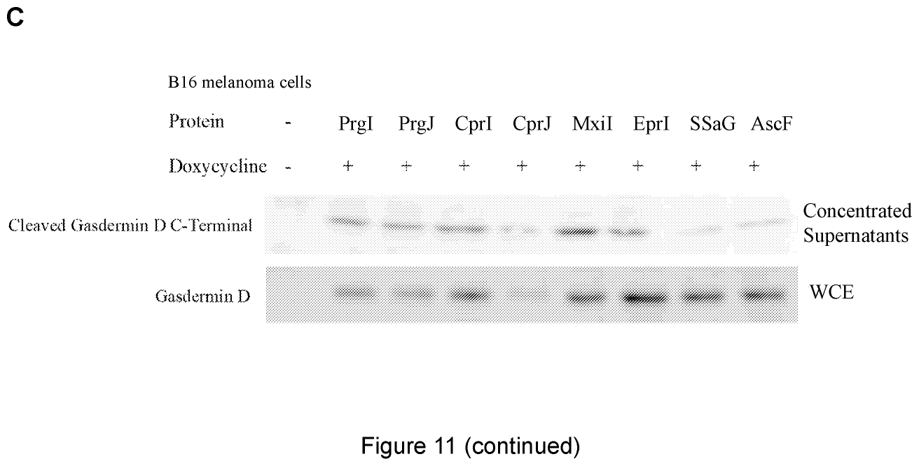

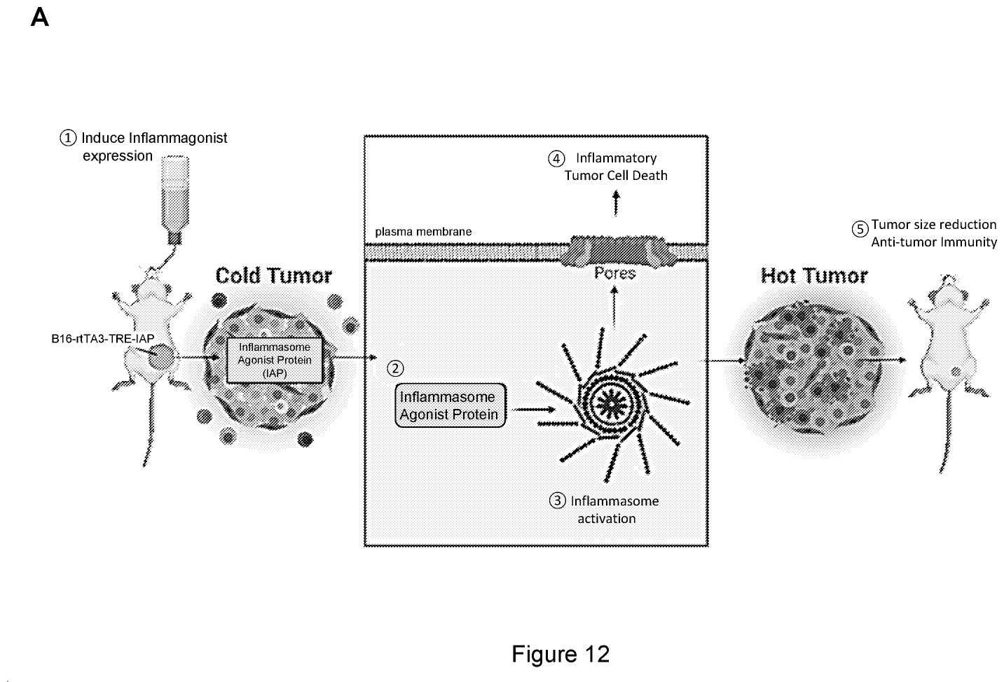

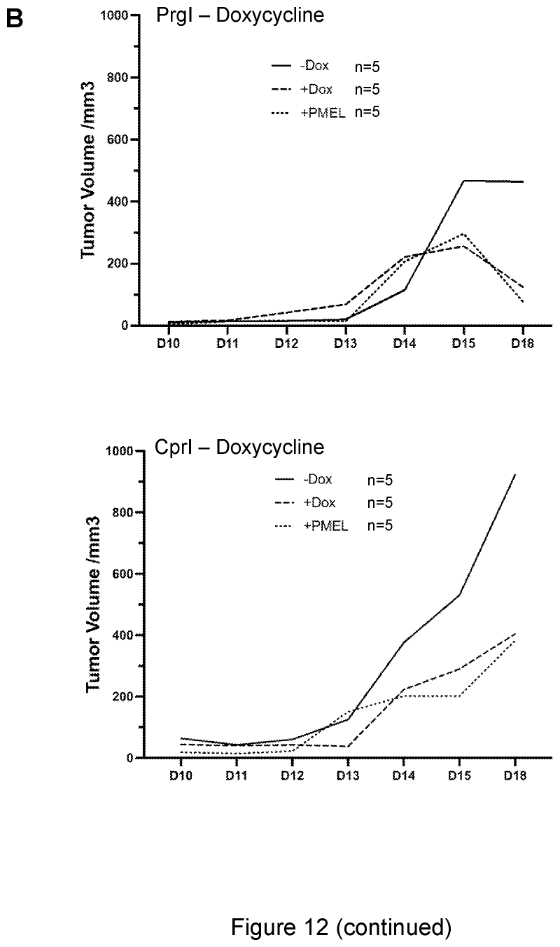

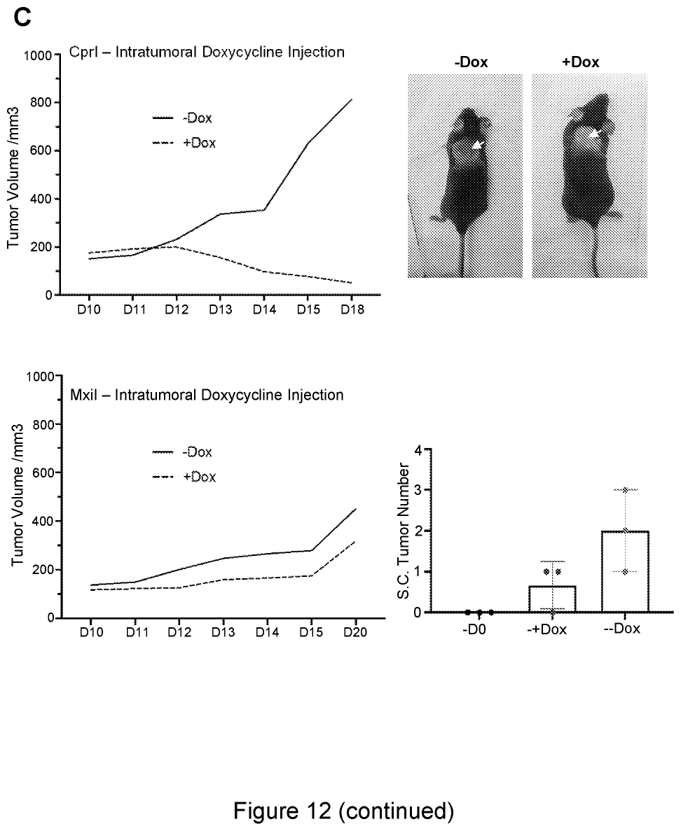

A fusion protein may comprise a Needle or Rod protein and an antigen, and optionally flagellin. For example, a fusion protein may comprise or consist essentially of i) a needle or rod protein; ii) an antigen; and iii) flagellin. The flagellin may be a full-length flagellin (e.g. comprising both TLR and NLR ligands) or a fragment comprising TLR or NLR ligand. A fusion protein may comprise or consist essentially of a needle or rod protein, and an antigen. Needle and rod proteins may be PrgI, PrgJ, CprI, CprJ, BsaK, MxiI, MxiH, YscF, EscF, PscF, EprI, and YscI. The antigen may be an antigen from a tumor or from a pathogen (e.g., bacteria, virus, fungus, or parasite). An example of an antigen derived from a pathogen is the major capsid (L1) protein of human papilloma virus (HPV) Type 11, shown in FIG. 11. A tumor antigen may be an antigen from a tumor including, but not limited to, ovarian, testicular, esophageal, melanoma, hepatocellular, and bladder cancer or any other cancer. An example of a tumor antigen in the fusion protein is NY-ESO-1, shown in FIG. 12. Examples of fusion proteins comprising needle or rod protein, tumor associated antigen, and optionally, flagellin include: TAA-[rod/needle protein]-flagellin; TAA-flagellin-[rod/needle protein]; flagellin-TAA-[rod/needle protein]; flagellin-[rod/needle protein]-TAA; [rod/needle protein]-flagellin-TAA; [rod/needle protein]-TAA-flagellin; TAA-[rod/needle protein]; or [rod/needle protein]-TAA.

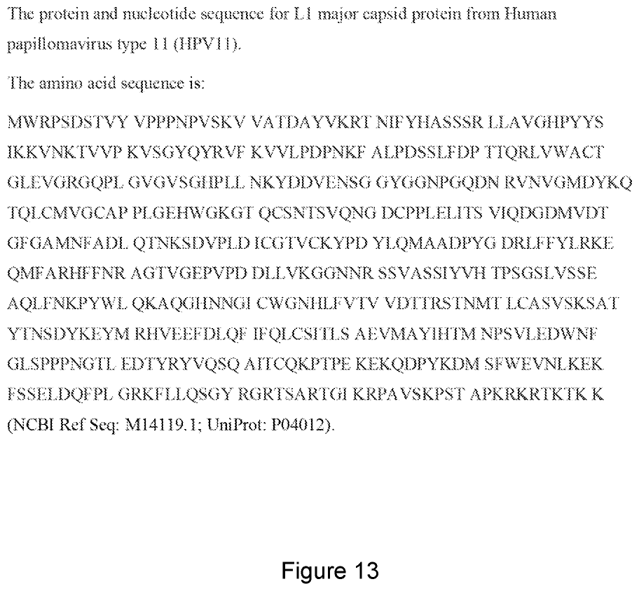

Fusion proteins may be chimeric or modified antibodies that target DCs and have fused thereto, or attached thereto, a Needle or Rod protein, a tumor antigen, and optionally flagellin or fragment thereof. The flagellin may be a full-length flagellin (e.g. comprising both TLR and NLR ligands) or a fragment comprising TLR or NLR ligand. For example, the antibody may be specific for DEC-205, CLEC9, or 33D1 present on the surface of DCs. An example of a chimeric antibody that comprises a V_{L}-V_{H} portion of an anti-DEC-205 antibody and a tumor antigen NY-ESO-1 (CDX-1401) is available from Celldex Therapeutics. Examples of fusion proteins comprising antibodies (or fragments thereof) specific for DCs, rod or needle proteins, optionally, flagellin or fragment thereof, and optionally, a tumor antigen are shown in FIG. 13. When the fusion antibody comprises a tumor antigen, a rod or needle protein, and flagellin, these proteins may be arranged in any order fused to the VH portion of the antibody. For example the arrangement may be (from N to C terminus) V_{L}-V_{H}-TAA-[rod/needle protein]-flagellin. Another arrangement may be V_{L}-V_{H}-TAA-flagellin-[rod/needle protein]. Another arrangement may be V_{L}-V_{H}-[rod/needle protein]-TAA-flagellin. Another arrangement may be V_{L}-V_{H}-[rod/needle protein]-flagellin-TAA. Another arrangement may be V_{L}-V_{H}-flagellin-[rod/needle protein]-TAA. Another arrangement may be V_{L}-V_{H}-flagellin-TAA-[rod/needle protein]. Another arrangement may be V_{L}-V_{H}-TAA-[rod/needle protein]. Another arrangement may be V_{L}-V_{H}-[rod/needle protein]-TAA. Another arrangement may be V_{L}-V_{H}-flagellin-[rod/needle protein]. Another arrangement may be V_{L}-V_{H}-[rod/needle protein]-flagellin. Linkers may be present between the various proteins in a fusion protein. Some of the linkers are shown in the examples in FIG. 13. A polyhistidine tag may also be present, as shown in some examples in FIG. 13 The flagellin may be a fragment thereof, such as that which is a TLR ligand. The rod or needle protein may be PrgI, PrgJ, CprI, CprJ, BsaK, BsaL, MxiI, MxiH, YscF, EscF, PscF, EprI, AscF, SsaG, and YscI.

The antibody or antibody fragment may be specific to DEC-205, CLEC9, or 33D1 and bind to the surface of DCs. Once bound to the surface of DCs, the antibody fusion proteins comprising Needle and/or Rod proteins activate the NLRC4 inflammasome and result in production of proinflammatory cytokines and an enhanced immune response.

All of the fusion proteins described herein may contain intervening (between two proteins) linkers or may be present without intervening linkers, and/or without the His-tag or with a different tag, such as a Flag-tag. This disclosure encompasses variants of the fusion proteins, wherein a variant of the fusion protein is at least 80%, 85%, 90%, 91%, 92%, 93%, 94%, 95%, 96%, 97%, 98% or 99% identical to the listed amino acid sequence. Variants of the fusion proteins may comprise a different order and/or sequence of the peptides in the N terminus- to -C terminus orientation and or nucleic acids described herein. For example, a fusion protein may comprise N-terminus-NY-ESO-1-PrgI-Flagellin-C terminus, but may comprise variants N terminus-Flagellin-PrgI-NY-ESO-1-C terminus and/or N terminus-PrgI-NY-ESO-1-Flagellin-C terminus.

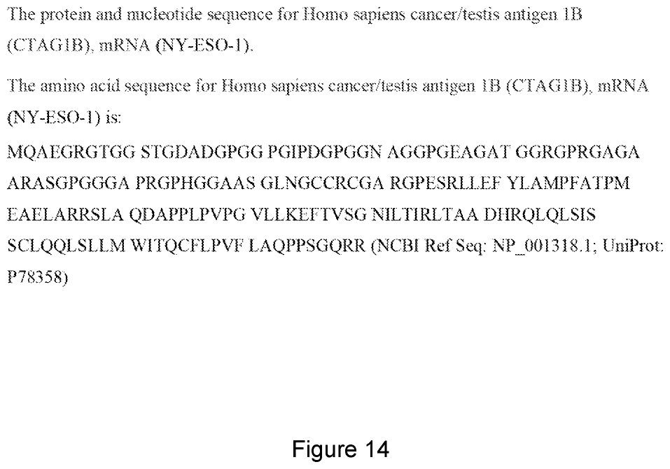

In an aspect, the disclosure provides nucleic acid sequences encoding the IAPs alone or as fusion proteins (or a variant thereof) as described herein (FIG. 14). The nucleic acid sequence can encode either a DNA or an RNA molecule corresponding to the amino acid sequence of the fusion proteins described above. In embodiments, the sequence which encodes an IAP or a fusion protein or a variant thereof, as described herein may have at least 80%, 85%, 90%, 91%, 92%, 93%, 94%, 95%, 96%, 97%, 98% or 99% identical to the listed nucleic acid sequences. This disclosure provides expression vectors comprising the sequences encoding fusion proteins as described herein. Methods for cloning expression vectors (fusion protein constructs), and methods for expressing and purifying recombinant fusion proteins of the invention are described in detail in Juleatt J. W. et al (2007) Vaccine (25)763-775. For example, vectors may also be expressed in tumor cells and immune cells, such as, dendritic cells (DCs), using retroviral, adenoviral, or lentiviral vectors. For generating fusion proteins, an expression construct encoding a fusion protein construct described or referenced in this disclosure may be introduced into the cell by transfection. Appropriate host cells include, but are not limited to, bacterial, yeast, insect, and mammalian cells. The host cells may then be lysed to extract the expressed polypeptide for subsequent purification.

The expression vector is not particularly limiting other than by a requirement for the fusion protein expression to be driven from a suitable promoter. Many suitable expression vectors and systems are commercially available. Examples of vectors include plasmids, cosmids, transposable elements, viruses (bacteriophage, animal viruses, and plant viruses), and artificial chromosomes (e.g., YACs). The expression vectors may be configured to produce fusion proteins. The fusion proteins may include components that facilitate purification, such as HIS or FLAG tag, or improve solubility or secretion or other functions. The vector may have a high copy number, an intermediate copy number, or a low copy number. Expression vectors typically contain one or more of the following elements: promoters, terminators, ribosomal binding sites, and IRES. A promoter may comprise one or more specific transcriptional regulatory sequences to further enhance expression and/or to alter the spatial expression and/or temporal expression of a nucleic acid. A nucleic acid encoding a fusion protein or chimeric/partially humanized/fully humanized antibody construct may also be operably linked to a nucleotide sequence encoding a selectable marker. A selectable marker may be used to efficiently select and identify cells that have integrated the exogenous nucleic acids. Selectable markers give the cell receiving the exogenous nucleic acid a selection advantage, such as resistance towards a certain toxin or antibiotic. Suitable examples of antibiotic resistance markers include those coding for proteins that impart resistance to kanamycin, streptomycin, neomycin, gentamycin (G418), ampicillin, tetracycline, chloramphenicol, puromycin, hygromycin, zeocin, and blasticidin.

Viral vectors suitable for introducing nucleic acids into cells include retroviruses, adenoviruses, adeno-associated viruses, rhabdoviruses, and herpes viruses. Examples of viral vectors that can be used in the present disclosure include, but are not be limited to, oncolytic viral vectors, AAV-FVIII/FIX, Lenti-FVIII, Lenti-FIX, Enadenotucirev, HSV HF10, Toca 511, Toca 511/FC, HSV G207, Gamma RV, MV-NIS, Oncolytic VV, NDV, LipoSFV-IL12, PANVAC-VF, NDV-TAA, VEE-PSMA, NDV, CVA21, CVA21+PLMab, LipoSFV-IL12, NDV PV701, and Lenti-hCEF-CT (See Diseases 2018 June, 6(2), the disclosure of which is incorporated herein by reference).

In a non-limiting embodiment, recombinant adeno-associated virus (rAAV) may be used. Given the benefit of this disclosure, the rAAV can be made by the skilled artisan using standard techniques and commercially available reagents. Suitable vectors that can be adapted to produce the rAAV are commercially available from, for example, the CLONTECH division of TAKARA BIO. In certain implementations plasmid vectors may encode all or some of the well-known rep, cap and adeno-helper components. The rep component comprises four overlapping genes encoding Rep proteins required for the AAV life cycle (Rep78, Rep68, Rep52 and Rep40). The cap component comprises overlapping nucleotide sequences of capsid proteins VP1, VP2 and VP3, which interact together to form a capsid of an icosahedral symmetry. Another plasmid providing the Adeno Helper function may also be co-transfected. The helper components comprise the adenoviral genes E2A, E4, or f6, and VA RNAs for viral replication.

In an aspect, this disclosure provides methods for delivery of proteins of the present disclosure and nucleic acid sequences encoding the proteins to cells of interest. Methods including viral and non-viral methods of nucleic acid transfer may be used to deliver polynucleotide sequences to cells. Sequences encoding fusion proteins of the disclosure can be delivered to tumor cells using known techniques. Such methods include, but are not limited to, transfection, microinjection, scrape-loading, and receptor-mediated uptake by the cell. Transfection may be transient or stable. Exemplary current methods of transfection include calcium phosphate precipitation, electroporation, lipofection, and peptide-mediated transfection. Ballistic DNA delivery and transduction (i.e., the introduction of foreign DNA by virus or virus vector infection) can also be used. For example, fusion proteins may be introduced wherein the polynucleotide encodes the fusion protein. Naked or protected mRNA may be used. mRNA encoding fusion proteins of this disclosure can be combined if desired with a delivery agent. Suitable delivery reagents for administration include but are not limited to Minis Transit TKO lipophilic reagent; lipofectin; lipofectamine; cellfectin; or polycations (e.g., polylysine), liposomes, nanoparticles, or combinations thereof. In embodiments, mRNA may be administered by an intratumoral injection. In embodiments, nanoparticles or other suitable drug delivery reagents may be used such that the mRNA is contained by the nanoparticles. The nanoparticles or other drug delivery reagent can be provided in association with a binding partner that binds specifically to, for example, a cancer cell marker or an immune cell marker.

In an embodiment, the construct encoding the fusion proteins of the present disclosure may be delivered to dendritic cells (DCs) ex vivo. The DCs may be obtained from tumor-bearing individual to be treated. For example, DC precursors may be obtained from an individual and differentiated (such as overnight) to provide DCs. Such techniques are well known (See, for example, U.S. Pat. No. 9,314,484, from which description is incorporated herein by reference). The fusion proteins of the present disclosure can be loaded on to DCs ex vivo, and then the DCs can be administered to individuals in need of treatment, via any of the various routes, such as intravenous, subcutaneous, or directly into a tumor-draining lymph node. An individual skilled in the art (such as a practicing/treating physician) will be able to determine the dosage and frequency of administration without undue experimentation. The DCs and the tumor antigen used in fusion proteins may be autologous to the individual. In additional embodiments, IAP mRNAs, constructs containing IAP mRNAs, DCs expressing IAP mRNAs, or any other IAP formulation described herein (such as IAP chimeric antibody fusion proteins) may be administered to an individual in need of treatment (such as an individual with cancer) in combination with other interventions, including standard chemotherapeutic treatments, radiation therapy, immune checkpoint inhibitors (e.g., anti-CTLA4, anti-PD-1/PD-L1) and/or other antibody-based or immunotherapeutic agents, and surgical interventions.

The present disclosure also provides pharmaceutical compositions comprising the adjuvants and/or fusion proteins as described herein. The compositions may comprise pharmaceutically acceptable diluents, preservatives, solubilizers, emulsifiers, and/or carriers. For example, pharmaceutical compositions may comprise various buffers (e.g., Tris-HCl, acetate, phosphate), additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). Such pharmaceutical composition components are known in the art See, e.g., Remington: The Science and Practice of Pharmacy (2005) 21st Edition, Philadelphia, PA. Lippincott Williams & Wilkins. The materials may be incorporated into particulate preparations of polymeric compounds such as polylactic acid, polyglycolic acid, etc. or into liposomes. Hyaluronic acid may also be used. Preparations for parenteral administration may include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain other adjuvants, preserving, wetting, emulsifying, and dispersing agents. The present compositions may be administered using any suitable route including parenteral, subcutaneous, intraperitoneal, intrapulmonary, and intranasal, and, if desired for local treatment, such as, intratumoral administration. Parenteral infusions include intramuscular, intravenous, intraarterial, intraperitoneal, intradermal, or subcutaneous administration.

In an aspect, this disclosure provides a method of enhancing an immune response to an antigen (e.g., tumor antigen) in an individual comprising delivering to the individual, a Needle and/or Rod protein (e.g., PrgI, PrgJ, CprI, CprJ, BsaK, BsaL, MxiI, MxiH, YscF, EscF, PscF, EprI, AscF, SsaG, and YscI), and a TAA (e.g., NY-ESO-1), and optionally, another adjuvant (e.g., TLR ligand, such as, flagellin). The needle or rod protein, TAA, and optionally, flagellin may be delivered as proteins or fusion proteins in pharmaceutical compositions, may be delivered as nucleic acid sequences (such as vectors or mRNA) that comprise sequences encoding these proteins or fusion proteins. The proteins or fusion proteins are then expected to be produced in vivo. Alternatively, the needle and/or rod protein, TAA and optionally flagellin may be exposed ex vivo to DCs obtained from an individual who needs treatment and then the loaded DCs can then be reintroduced into the host. Based on the data provided in this disclosure, it is considered that the Needle and Rod proteins enhance the immune response elicited by cells of the immune system, including, but not limited to, dendritic cells, by one or more of the following: activating the NLRC4 inflammasome, producing inflammatory cytokines (e.g., IL-1β and IL-18), inducing pyroptosis, stimulating CD4⁺ and/or CD8⁺ T cell responses, and increasing Ag-specific Ab production.

The present disclosure provides methods to exploit the inflammasome pathway of innate immune defense to modulate the microenvironment of a tumor within an individual to shift it from an immunosuppressive to an immuno-stimulatory microenvironment. Messenger RNAs encoding the inflammasome agonist proteins may be directly delivered intra-tumorally to elicit inflammatory conditions that drive dendritic cells and T cells to target tumor cells for killing and removal. The messenger RNA molecules comprising the open reading frames of Needle and Rod protein encoding genes are codon optimized, subjected to pseudo-Uridine nucleoside modifications and 5′ capping. This approach will enable intra-tumoral expression of the IAPs such as Needle proteins PrgI and CprI to elicit a vigorous inflammatory immune response by engaging the inflammasome. The protein nature of these inflammasome agonists sets them apart from other inflammasome agonists of non-protein nature, and makes them uniquely amenable to adaptation for an mRNA-based therapeutic platform. This approach may serve as a therapeutic strategy to remodel the tumor microenvironment in individuals with different types of solid tumors.

The present disclosure provides vaccine compositions. The term “vaccine” refers to a composition that can be used to elicit protective immunity in a recipient. It should be noted that to be effective, a vaccine of the invention can elicit immunity in a portion of the immunized population, as some individuals may fail to mount a robust or protective immune response, or, in some cases, any immune response. This inability may stem from the individual's genetic background or because of an immunodeficiency condition (either acquired or congenital) or immunosuppression (e.g., due to treatment with chemotherapy or use of immunosuppressive drugs). Vaccine efficacy can be established in animal models. The compositions comprising Needle and/or Rod proteins and a TAA may be administered in a single shot or may be followed up by booster shots. Such administration regimens are routine and well within the purview of those skilled in the art. The TAA may be administered simultaneously with Needle and Rod protein adjuvants, or the two may be administered separately. Similarly, when flagellin is used, it may be administered separately, or together with a needle or rod protein or TAA.

The compositions and methods of the present invention are useful for inducing an anti-tumor immune response, such as for treatment of cancers. Examples of cancer include, but are not limited to, carcinoma, lymphoma, blastoma, sarcoma, and leukemia or lymphoid malignancies. More particular examples of cancers include: squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer (including gastrointestinal cancer, pancreatic cancer), glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer. A cancer includes primary malignant cells (e.g., those that have not migrated to sites in the subject's body other than the site of the original malignancy) and secondary malignant cells (e.g., those arising from metastasis, the migration of malignant cells to secondary sites that are different from the site of the original tumor).

In embodiments, administering the described agents to an individual in need thereof exhibits an improved activity relative to a control. In an embodiment, the control comprises different IAP than the one eliciting the particular effect, a different form of the IAP, such as with a different binding partner and/or TLR ligand and/or cancer antigen, than the particular construct that is delivered. In embodiments, administering a described agent to an individual may elicit a synergistic effect, including but not necessarily limited to a synergistic anti-cancer effect, or a synergistic adaptive or cell-mediated immune response, or a combination thereof.

Immune responses may be measured by the proportion of antigen-specific CD8⁺ T cells that are produced. To that end, some embodiments of the present invention comprise methods to enhance the production and or activation state of antigen-specific CD8⁺ T cells (e.g., tumor antigen-specific CD8⁺ T cells, such as NY-ESO-1-specific CD8⁺ T cells) in an individual comprising administering to the individual an effective amount of a composition of the present disclosure, whereby the composition acts as an adjuvant or includes an adjuvant component to enhance the numbers of antigen-specific CD8⁺ T cells.

Immune response may be measured by the amount of inflammatory cytokines produced. Some embodiments of the present invention comprise methods to enhance the production of proinflammatory cytokines in an individual comprising administering to the individual an effective amount of a composition of the present disclosure, whereby the composition acts as an adjuvant or comprise an adjuvant to enhance the production of proinflammatory cytokines. In an example, the proinflammatory cytokines (e.g., IL-1β) are produced by the inflammasome in immune cells.

Immune response may be measured by the proportion of antigen-specific antibodies that are produced. As such, the present disclosure describes methods to enhance the production of antibodies to an antigen in an individual comprising administering to the individual an effective amount of a composition of the present disclosure, whereby the composition acts as, or comprises, an adjuvant to enhance the production of antigen-specific antibodies. In other embodiments, the Needle and Rod proteins, fusion proteins, and/or chimeric Ab fusion proteins stimulate T follicular helper cells to aid B cells in producing a strong antigen-specific antibody response.

In an aspect, this disclosure provides methods of activating dendritic cell comprising: (a) isolating CD14⁺ monocytes from the peripheral blood of an individual; (b) stimulating the CD1⁺ monocytes for an appropriate amount of time with a suitable amount of IL-4 and Granulocyte Macrophage Colony Stimulating Factor (GM-CSF) to differentiate the monocytes into dendritic cells; (c) contacting the DCs with a Needle and Rod protein, and optionally flagellin, and/or an antigen as a fusion protein with an antibody having specificity for DC surface protein, or with a vector encoding a Needle/Rod protein, optionally, flagellin, and TAA—separately or as fusion protein; and after waiting a sufficient amount of time, (d) measuring DC activation, inflammasome activation, and/or cytokine production; and (e) administering activated DCs to the individual in need of treatment.

In embodiments, this disclosure provides a method for enhancing an immune response to a desired antigen in an individual comprising administering to the individual DCs that have activated inflammasome, with the DCs having been contacted with one or more Needle and/or Rod proteins and/or fusion proteins as described herein. In embodiments, the disclosure comprises antigen presenting cells, such as dendritic cells, which have been exposed to at least one polypeptide fusion protein described herein. In performance of this embodiment, the dendritic cells may first be isolated from an individual using conventional techniques. The dendritic cells may be isolated from the individual in whom an enhanced immune response to a desired antigen is intended. The isolated dendritic cells may be also exposed to a fusion protein described herein, such as by pre-loading the dendritic cells with the fusion protein. The isolated dendritic cells can be administered to the individual so as to elicit an enhanced immune response to the desired Ag. In an embodiment, the DCs that have been contacted with the fusion proteins of the present disclosure can be mixed with CD4⁺ and/or CD8⁺ T-cells. The DCs activated by the fusion proteins, such as a fusion proteins with a TAA (e.g., NY-ESO-1), may in turn activate the T cells. These activated T cells can then be separated from the DCs and administered to an individual in need of treating. In an example, the activated T cells may be specific for a TAA, such as NY-ESO-1, and the individual in need of treating has a tumor that is expressing NY-ESO-1.

In embodiments of the disclosure, methods for inducing an anti-tumor immune response in a mammal or for treating a cancer are provided, with the methods comprising administering to a mammal or patient an immunogenically effective amount of a composition comprising a DC, wherein the DC is expressing a fusion protein or has been stimulated by a fusion protein of the present disclosure. DCs are known to be potent stimulators of the adaptive immune response (e.g., T and/or B cell mediated immune responses) and may be used to generate tumor-specific T cells, such as NY-ESO-1-specific, CD8⁺ T cells.

The fusion proteins of the present disclosure, or a composition comprising one or more fusion proteins may be delivered to dendritic cells to activate them to enhance an immune response. It is considered that activation of the DCs by the fusion proteins described in this disclosure (e.g., fusion proteins comprising a needle or rod protein, TAA, optionally, flagellin, and a DC specific antibody) results in the formation and activation of the NLRC4 inflammasome. The activated NLRC4 inflammasome produces proinflammatory cytokines that contribute to enhancing immune responses. The DC activation may be achieved in vitro or in vivo. As such, the present compositions may be administered to an individual who is in need of an enhanced immune response, such as an individual afflicted with cancer or an infection. Examples of conditions in which the present fusion proteins can be used is any condition in which an immune response is abnormal or insufficient. Such conditions can include, but are not limited to, cancer and infectious diseases. In embodiments, treatment of a patient with a vaccine, fusion protein, or other formulation (e.g., mRNA encoding IAPs) described herein can be combined with other interventions, including standard chemotherapeutic treatments, radiation therapy, immune checkpoint inhibitors (e.g., anti-CTLA4, anti-PD-1/PD-L1) and/or other antibody-based or immunotherapeutic agents, and surgical interventions.

In an aspect, the disclosure provides methods for treating an individual having or suspected of having cancer comprising (a) obtaining a sample of the individual's tumor (e.g., a biopsy sample) and determining the presence of a TAA, and if the TAA is present then b) isolating the TAA; and c) administering to the individual a suitable amount of a composition comprising Needle and/or Rod proteins and the TAA or a fusion protein comprising Needle and/or Rod proteins and the TAA, or administering to the individual DCs that have been exposed to a fusion protein comprising a needle or rod protein, the TAA, and optionally, flagellin, and a DC specific antibody.

In an embodiment, the individual's tumor sample has NY-ESO-1 expressing cells or any other suitable TAA. A sample from the individual's tumor (e.g., biopsy and/or blood sample) may be used to measure NY-ESO-1 and/or additional TAA markers. If the tumor is found to have elevated NY-ESO-1 and/or other suitable TAA levels, a suitable amount of the fusion proteins described in this disclosure can be administered to the individual using a suitable route (e.g., intratumoral, intravenous, intradermal injection). The treatment may be carried out without first sampling NY-ESO-1 and/or other suitable TAA levels.

In an embodiment, the invention provides a method for inducing an anti-tumor immune response in a mammal comprising administering to said mammal in need thereof an immunogenically effective amount of a composition comprising a fusion protein, wherein said fusion protein comprises one or more Needle and/or Rod proteins and a TAA.

In an embodiment, the disclosure provides a method for treating a cancer in a patient comprising administering to said patient in need of such treatment a composition comprising a fusion protein, wherein said fusion protein comprises one or more Needle and/or Rod proteins and a TAA or one or more Needle and/or Rod proteins, flagellin, and a TAA, in an effective amount for eliciting an anti-tumor immune response.

In an embodiment, the disclosure provides a method for inducing an anti-tumor immune response in a mammal comprising administering to said mammal in need thereof an immunogenically effective amount of a composition comprising DCs expressing a fusion protein, wherein said fusion protein comprises one or more Needle and/or Rod proteins and a TAA or one or more Needle and/or Rod proteins, flagellin, and a TAA.

In an embodiment, the invention provides a method for treating cancer in a patient comprising administering to said patient in need of such treatment a composition comprising DCs expressing a fusion protein, wherein said fusion protein comprises one or more Needle and/or Rod proteins and a TAA or one or more Needle and/or Rod proteins, flagellin, and a TAA in an effective amount for eliciting an anti-tumor immune response.

In an embodiment, the present disclosure provides a method for reducing the size of a tumor or arresting the growth of a tumor or reducing the rate of growth of a tumor or reducing any other symptom that is associated with an individual being afflicted with the tumor—all of which are considered as “treatment”—comprising administering to an individual in need of treatment, a therapeutically effective amount of a composition comprising Needle and/or Rod proteins or fusion proteins as described herein. The treatment may be in the form passive immunization.

In an aspect, this disclosure provides kits for the treatment of cancer. A kit may comprise a composition comprising a needle or rod protein and flagellin or another adjuvant. Optionally, a tumor associated antigen may also be included. Instructions for use may also be included.

The following examples are meant to illustrate, and are not intended to be limiting.

### EXAMPLE 1

This example describes the development of fusion proteins comprising one or more Needle and/or Rod proteins, activation of the inflammasome by fusion proteins, and delivery of fusion proteins to dendritic cells to modulate immune responses.

Family of Needle and Rod proteins as novel inflammasome agonists

Methods

Cloning and Expression of Type III Secretion System (T3SS) Needle and Rod Proteins

The Needle and Rod protein family members include but not limited to PrgI, PrgJ, CprI, CprJ, BsaK, MxiI, MxiH, YscF, EscF, PscF, EprI and YscI. The first members we have tested are PrgI and PrgJ. We call these our founder proteins. We have also tested PrgI, PrgJ, CprI, CprJ, BsaK and MxiI in a second round. The larger family of needle-like proteins also include the following Needle proteins, including MxiH, YscF, EscF, PscF, EprI, that form the needle of the injection apparatus, as well as YscI. MxiH is an extracellular alpha helical needle that is required for translocation of effector proteins into host cells, and once inside, the effector proteins subvert normal cell function to aid infection. YscI (Yop proteins translocation protein I) in Yersinia is involved in the translocation of Yop proteins into the host cell cytosol analogous to Salmonella PrgJ.

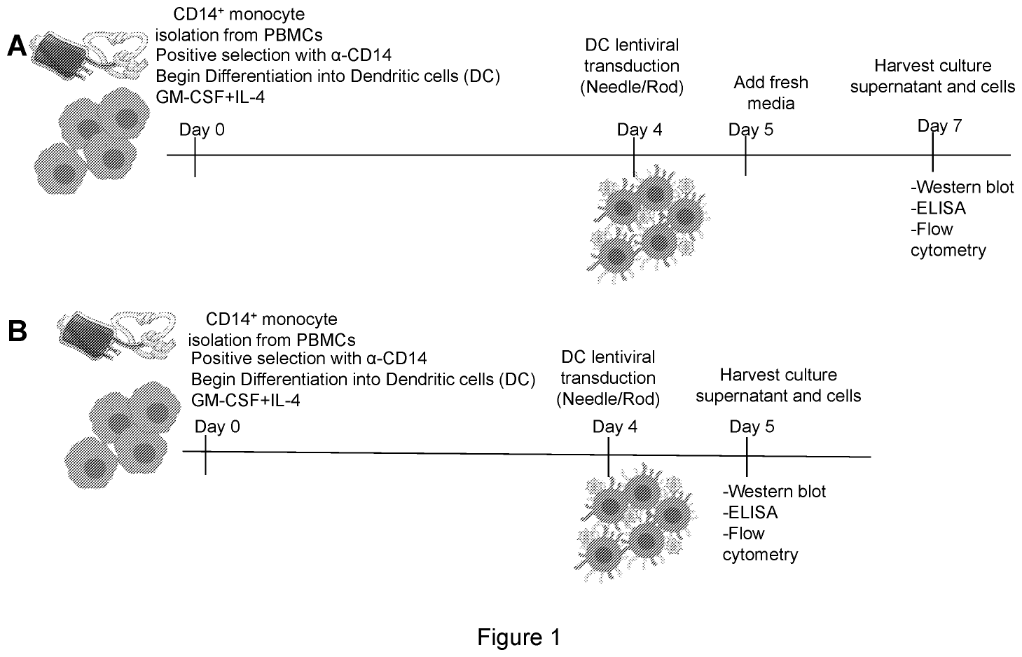

The coding sequences of the PrgI and PrgJ proteins of Salmonella typhimurium; CprI and CprJ proteins of Chromobacterium violaceum (strain MK); BsaK protein of Burkholderia thailandensis (strain E264); and MxiI protein of Shigella flexneri (strain M90T) were downloaded from NCBI. CprI and CprJ sequences were further modified by GeneScript. The synthesized sequences were cloned into the bidirectional lentiviral vector (BdLV-3000) vector, and in between the XmaI and SalI sites (FIG. 1). BdLV-3000 exploits the intrinsic bidirectional activity of the PGK promoter to drive divergent transcription of two transgenes although a minimal CMV promoter is also upstream of the GFP reporter. A Flag tag was introduced into the N-terminus of these constructs for checking protein expression.

Rod protein BsaK from Burkholderia species such as B. thailandensis and B. mallei and Rod protein PrgJ from Salmonella species are bacterial effector proteins injected into the host cell cytoplasm to elicit activation of the inflammasome. In murine cells, the protein NAIP2 serves as the specific inflammasome receptor for T3SS rod proteins PrgJ and BsaK. Indeed, BsaK was shown to interact with murine NAIP2, but not NAIP1, NAIP5, NAIP6 and NLRC4 and elicit the cleavage of pro-IL-1β, thereby establishing NAIP2 as the specific murine receptor for the T3SS rod proteins. Human cells do not express NAIP2 and only express one member of the NAIP family called human NAIP (hNAIP). It is for this reason that the human U937 monocytic cell line was found to not be responsive to bacterial effector Rod proteins PrgJ and BsaK. However, it is conceivable that human cell types other than monocytes, macrophages and dendritic cells might be able to respond to Rod proteins through receptors like hNAIP that are yet to be identified.

Needle CprI and Rod CprJ are bacterial effector proteins injected into the cytoplasm of the host cells via the T3SS of C. violaceum strain MK (ATCC 12472), a virulence factor comprised of a needle and syringe like multi-protein apparatus that assembles on the plasma membrane of targeted host cells. In mouse models, the NLRC4 inflammasome is important for the control of infections with both C. violaceum and B. thailandensis. CprI is a sequence paralog of CprJ, and it has been shown that human macrophages detect the needle protein CprI and not rod protein CprJ by the human protein hNAIP as the inflammasome receptor. hNAIP engagement by CprI promotes the oligomerization of the Nod-like receptor protein NLRC4, activation of the NLRC4 inflammasome to elicit caspase-1 activation and cleavage, and the subsequent cleavage of pro-IL-1β and the induction of pyroptosis, an inflammatory form of cell death.

Lentiviral (lv) Production

LV were produced by co-transfection of 293T cells with the recombinant lentiviral BdLV plasmids encoding needle or rod proteins, 2) the human immunodeficiency virus (HIV) packaging plasmid 8.9, and 3) the envelope plasmid (VSV-G). 293 T cells were plated on amine-coated petri-dishes, in order to ensure the 80-90% of confluence for transfection. The vector, the packing 8.9 and the envelope plasmid, were combined with water and then, CaCl2 (Sigma-Aldrich, St Louis, MO) and was added to Hepes buffer saline (HBS) buffer (20 mM Hepes, 280 mM NaCl, 10 mM KCl, 1.5 mM Na2HPO4, 12 Nm D-glc pH 7). After 5-minute incubation, the mixture was added to the cells. Media was replaced by Optimen supplemented with Hepes (Gibco, Invitrogen, Auckland, New Zeland) and penicillin/streptomycin (Gibco, Invitrogen, Auckland, New Zealand) 18 h post-transfection. Viral supernatant was harvest 48 h post-transfection, centrifuged 5 minutes at 1500 rpm and filtered through 0.45 μm filter. Low speed concentration of the vector was performed overnight at 3000 g at 4° C., and the concentrated virus (100×) were collected the following morning.

Human Dendritic Cell (DC) Isolation

Peripheral blood was purchased from the New York blood bank. De-identified leukopack bags containing between 30-50 ml were diluted in PBS (1:2) and then added to 15 ml of Ficoll for gradient separation. White blood cells were recollected and centrifuged for different times to separate platelets and the rest of blood cells. The peripheral blood mononuclear cells (PBMCs) were counted, usually getting around 6×108-2×109 cells. The cells were incubated with CD14 magnetic beads from Miltenyi and were separated positively on magnetic columns. The number of CD14 cells recovered was typically around 6×107-6×108 cells. Purity of the isolation was measured by flow cytometry, staining the cells with CD14 antibody obtaining a purity between 80-95%. The cells were then centrifuged and resuspended in differentiation media (RPMI+IL-4+GM-SCF) at a concentration of 5×106cells/ml in 6 well plates. Fresh media (RPMI+IL4+GM-SCF) was added to each well (3 ml to each well) to make a final volume of 5 ml at day 3.

Dendritic Cell Transduction

On day 4 after the isolation, cells were collected, centrifuged (10 min 300 g) and counted. The numbers were around 1-9×107 cells. The minimum number of cells per condition was set at 1×107 cells to conduct all the Western blot analyses. At least 1×10⁷ cells were thus plated in 12 well plates (one well per condition) in Optimen+IL4+GM-SCF media. The transduction volume was 500 μl. The recombinant lentiviruses were then added (30 μl of a 109 IU/ml virus). DCs were left either 72 hours or 20 hours with the virus before re-collection:

For the 72 hours protocol, cells where left with the virus overnight and first thing the next morning (day 5), fresh media (Optimen+IL4+GM-SCF) was added to the transduction (500 μl). On day 7, 500 μl of the media was collected for ELISA and flow cytometry. The rest was used for supernatant concentration and whole cell extraction for Western blot.

For the 20-hour protocol, cells were incubated with the virus 5 hours. Then fresh media was added. The next day, day 5, 500 μl of the media was collected for ELISA and flow cytometry. The rest is used for supernatant concentration and whole cell extraction for western blot.

ELISA

IL-1β and TNFα were measured in the supernatants of cells. Plates were coated with the capture antibody (anti-h TNFα Clone 28401 #MAB610 R&D system) the day before the recollection of the supernatant and left overnight at 4° C. The next day the plate were blocked with PBS+5% BSA for 2 hours and then 50 μl of the sup was added to each well. The plate was incubated overnight at 4° C. The following morning the plate was wash and incubated with the detection antibody (TNFα biotinylated-#BAF2010 R&D system) for 2 hours followed by incubation with the HRP-streptavidin antibody (Biolegend-#405210) for 1 hour. KPL SureBlue TMB Microwell peroxidase substrate (Sera care #5120-0075) solution was added and incubated for 20 minutes followed by the KPL TMB stop solution (Sera care-5150-0020). For IL-1β detection the Human IL-1β/IL-1F2 DuoSet ELISA (R&D system-#DY201) was used. The absorbance of the plate was then read in the Spectra Max Id3 at 492 nm for IL-1β OR 540 nm for TNFα.

Flow Cytometry

Cells where collected and washed with PBS at 300 g for 10 minutes and then resuspended in the DC marker cocktail at concentration 1:100 together with human Fc-block (BD Pharmigen-#564219) to check their differentiation to DCs. The marker used were obtained from biolegend: DC-SIGN(DC209)-BV421 (clone 9E9A8-#330118); Dec-205 (CD205)-PE (clone HD30-#342204) and from Invitrogen: CD14-APC-eFluor 780 (clone 61D3-#41-0149-42); HLA-DR-APC (clone LN3-#17-9956-42); CD40-PE 8 (clone 5C3-#12-0409-42); CD83-PerCP-efluor710 (clone-HB15e-#46-0839-42); CD11c-efluor506-(clone 3.9-#69-011642). After the cells had been marked with the DC cocktail, they were washed with PBS at 300 g for 10 minutes, they were stained with SYTOX™ Red Dead Cell Stain (#S34859) for 15 minutes and then were measured by flow cytometry to determine pyroptosis (cell death).

qPCR

RNA was isolated from 10⁶ 293 T cells, DCs and THP1 cells with the RNeasy Mini Kit (Qiagen-#74104) following manufacturer's instructions. RNA quantity was measured by nanodrop. 500 ng of RNA was used for reverse transcription. Oligo dT (Invitrogen-#18418020) and dNTPs are added and the mix is heat for 5 min at 65° C. The tube is then incubated with DTT (Invitrogen-#y00147), RNaseOut (Invitrogen-#10777019) and SuperScript III RT (Invitrogen-#56575) at 50° C. for 1 h and then at 70° C. for 15 minutes to inactivate the reaction.

The RT product is diluted to a final concentration of 5 ng/μl and the TaqMan Fast Advance Mastermix (Applied BioSystems-444554) together with the primer and probe mix is added (NLRP3-mM00840904 TRL5-hS00152825; NAIP-HS3037952; GAPDH-hS03929097; NRLC4-hS00368367).

Concentration of DC Supernatants Using TCA Precipitation

Supernatants from around 2,000,000 human DCs per condition (prepared in serum-free Opti-MEM) were harvested 20 hours or 72 hours following viral transduction. Supernatants proteins were concentrated using Trichloroacetic acid (TCA) precipitation (with ¼ V/V TCA), pelleted by centrifugation at 16,000 g, and washed in cold acetone. Precipitated proteins were then resuspended and denatured in 2×Laemmli buffer containing 1% β-mercaptoethanol for 5 minutes at 95° C., before SDS-PAGE resolution on 12% polyacrylamide gels.

Preparation of DC Whole Cell Extracts (WCE)

Around 2,000,000 human DCs per conditions were harvested 20 hours or 72 hours following viral transduction and washed in cold PBS. Cells were lysed in 50 mM Tris-HCl (pH 7.9), 300 mM NaCl, 1% Triton X-100, supplemented with protease inhibitor and phosphatase inhibitor cocktails (respectively Complete Protease and Phostop, Roche). Whole cell extracts were cleared by centrifugation at 16,000 g for 10 min at 4° C. Protein concentrations were determined using the Bradford method. Samples were denatured in Laemmli buffer containing 1% β-mercaptoethanol for 5 minutes at 95° C., before SDS-PAGE resolution.

Western Blots

After SDS-PAGE electrophoresis of whole cell extract and concentrated supernatants, proteins were transferred onto a 0.45 μm PVDF membrane (Millipore). Membranes were blocked with 7% evaporated milk in PBS 0.2% Tween and were incubated with primary antibodies and peroxidase-conjugated secondary antibodies (all diluted in PBS 7% milk 0.2% Tween). Bound antibodies were visualized using the Amersham™ ECL or Pierce® ECL2 detection reagents, and imaged using Amersham™ Imager 600 (GE Healthcare).

Antibodies for Western Blots Analysis

Antibodies for Western Blot were obtained from: 1) Cell Signaling Technology: anti-β-Actin (#3700), anti-Caspase-1 (#3866), anti-Caspase-5 (#48680); 2) From Santa Cruz Technologies: anti-ASC (#sc-22514-R) ; 3) From Sigma Aldrich: anti-GasderminD (#G7422), 4) From Research & Development: anti-IL-1β (AF-401-NA).

Measurement of Lactate Dehydrogenase (LDH) Release by DCs

Cell death of human DCs was measured using the Cytotox96 cytotoxicity assay (Promega) following manufacturer's instructions. The assay measures the release of lactate dehydrogenase (LDH) into the supernatant calculated as the percentage of maximum LDH content, measured from total cellular lysates (100%).

Results are provided in FIGS. 1-10.

Results are provided in FIGS. 1-10.

To test whether Needle and Rod proteins activate the inflammasome in primary human dendritic cells, we resorted to expression of these proteins in dendritic cells through lentiviral transduction. We used the bidirectional lentiviral reporter lentiviral vector platform BdLV. This lentiviral vector coordinately expresses green fluorescent protein (GFP) under control of the CMV promoter and the cloned insert (Needle and Rod proteins in this case) under control of the hPGK promoter. As examples, we have cloned into BdLV the cDNAs encoding flagellin, the Needle protein PrgI from Salmonella typhimurium, the Rod protein PrgJ from Salmonella typhimurium, and cDNAs encoding fusion proteins of flagellin and PrgI and flagellin and PrgJ. Maps of these recombinant lentiviral vectors are shown in FIG. 1.

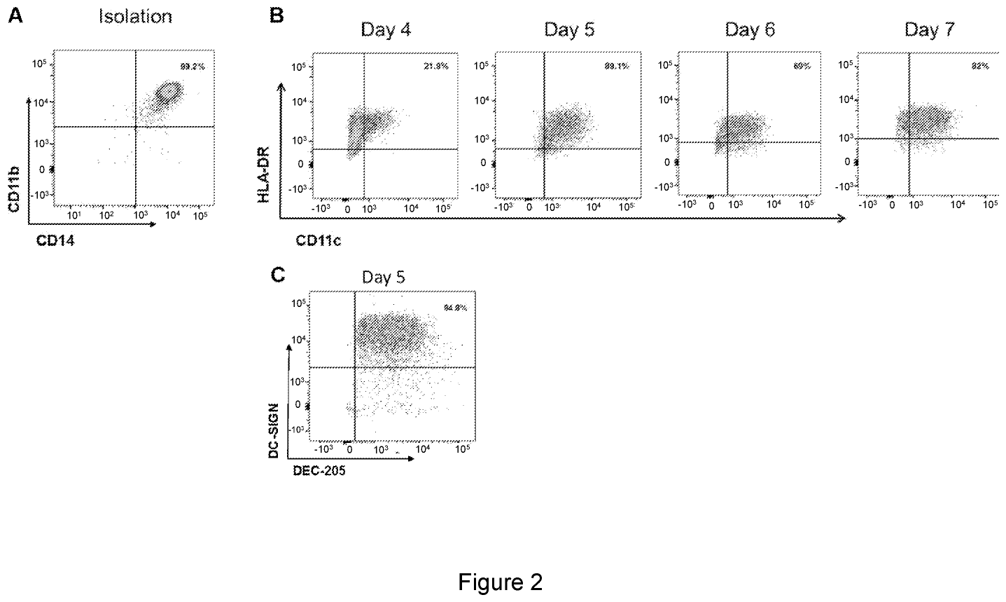

We differentiated dendritic cells (DC) in vitro from CD14⁺ monocytes derived from peripheral blood leukopacks (FIG. 1). The cells were 99% monocytes at the start of the culture (FIG. 2A). Over the course of seven days of culture, we found enrichment in cells with surface expression of CD11c and HLA-DR, markers expressed at high levels on DC (FIG. 2B). These cells also expressed the DC markers DC-SIGN and DEC-205 beginning on day 5 of culture (FIG. 2C). The expression of DEC-205 was particularly noteworthy because it would enable targeting DEC-205 on these cells for delivery of chimeric antibody proteins with specificity to DEC-205 (see embodiments 40 and 90 as examples).

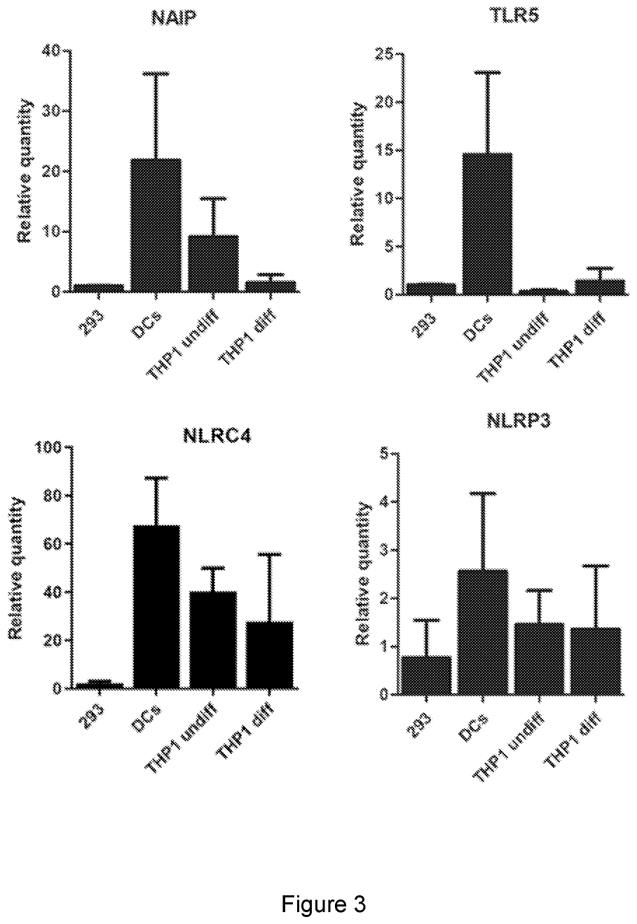

We next examined the expression of transcripts encoding for components of inflammasomes in primary human DC, in undifferentiated THP-1 cells that represent monocytes, and in PMA-differentiated THP-1 cells that represent macrophages (FIG. 3). We found that highest expression levels of transcripts encoding for the flagellin and Needle protein sensor NAIP, the flagellin recognizing Toll-like receptor TLR5, the Nod-like receptor that responds to flagellin and Needle protein, NLRC4, and finally the Nod-like receptor NLRP3 that comprises the apical component of the canonical inflammasome that responds to multiple stimuli including LPS+ATP. Monocytic THP-1 cells had higher levels of NAIP transcripts than macrophage THP-1 cells while the levels of expression of NLRC4 and NLRP3 were similar in macrophage and monocytic THP-1 cells (FIG. 3). The levels of TLR5 transcripts were low in THP-1 cells (monocyte or macrophage) compared to primary human DC (FIG. 3).

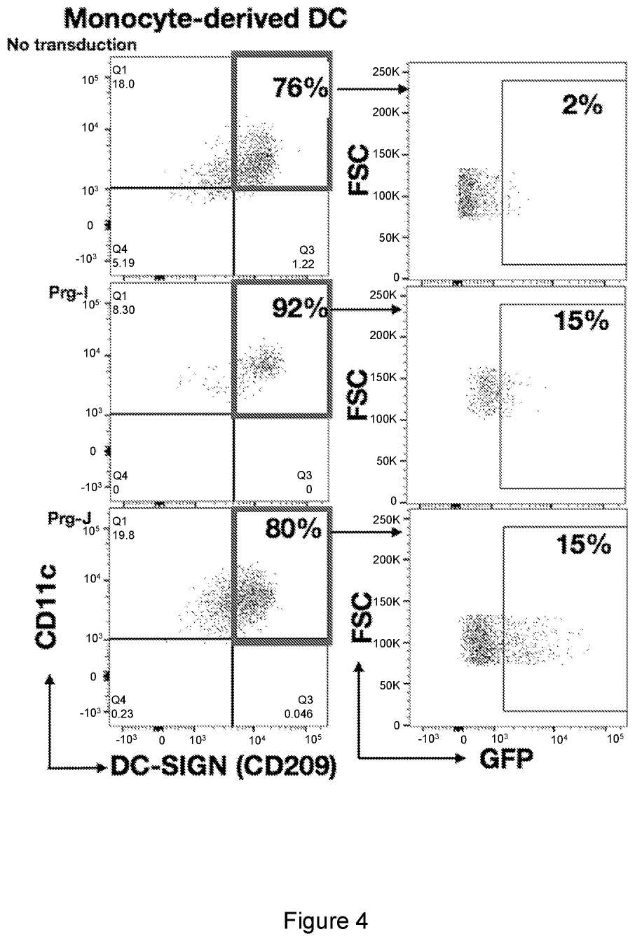

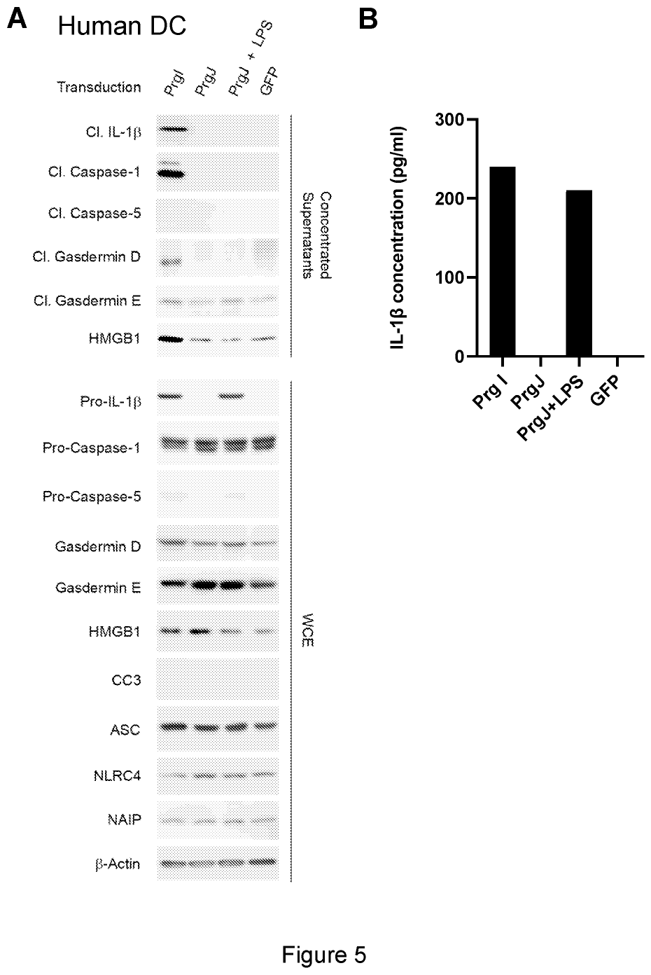

Human DC cultures were transduced on day 4 with recombinant lentiviruses expressing either PrgI or PrgJ (as indicated in the schematic in FIG. 1). GFP served as the reporter of lentiviral transduction efficiency whereby 15% of CD11c⁺ DC-SIGN⁺ cells within the DC cultures expressed GFP and were thus successfully transduced (FIG. 4). At 72 hours post-transduction or 7 days from the initiation of the human DC differentiation cultures, we harvested the cells for preparation of whole cell extracts to conduct Western blots for various proteins involved in cell death and inflammasome activation. We also harvested the culture supernatants setting aside a small aliquot for conducting ELISAs for measurement of IL-β protein and we concentrated the proteins within the rest of the supernatants to conduct Western blot for detection of the cleaved forms of IL-1β and caspases 1 and 5 as well as cleavage of Gasdermin D and E and the presence of HMGB-1. These analyses confirmed expression of NLRC4 and NAIP in primary human DC (FIG. 5A) consistent with the expression of the transcripts for these proteins (FIG. 3), as well as the expression of the inflammasome adaptor ASC, Gasdermin D, Gasdermin E, and HMGB-1. We also detected the pro-forms caspase-1, but we did not detect the pro-form of caspase-5 which has been reported to serve as the cytosolic receptor for LPS. Interestingly, PrgI and not PrgJ induced the expression of pro-IL-1β, whereas the addition of LPS to PrgI was necessary to induce the expression of pro-IL-1β (FIG. 5A, WCE panels). Only PrgI and not PrgJ or PrgJ+LPS elicited inflammasome activation manifested by the cleavage of IL-1β and caspase-1. Detection of the cleaved forms of Gasdermin D and Gasdermin E, as well as the increased levels of HMGB-1 in the culture supernatants were indicative of pyroptosis, an important downstream consequence of inflammasome activation that elicits further inflammation (FIG. 5). We did not detect cleavage of caspase-3 within the WCE after any of the stimuli indicating that the cells were not undergoing apoptosis. This was particularly important to note in the PrgI transduced DC indicating cell death by an inflammatory form of cell death, pyroptosis rather than non-inflammatory apoptosis. GFP expression alone through the empty lentiviral vector did not elicit the expression of pro-IL-1β or inflammasome activation.

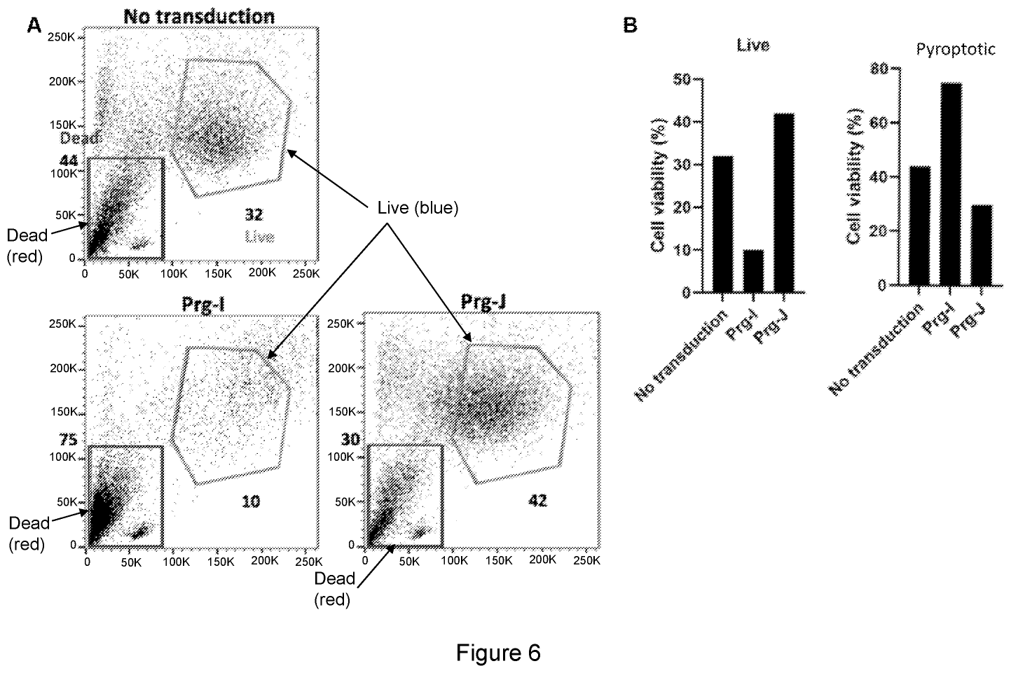

Further characterization of pyroptosis in the human DC cultures showed an increased percentage of PrgI transduced DC (75%) had undergone pyroptosis by 72 hours compared to the background dead cells within these cultures (44 and 30% in non-transduced and Prg-J transduced DC) (FIGS. 6A and 6B).

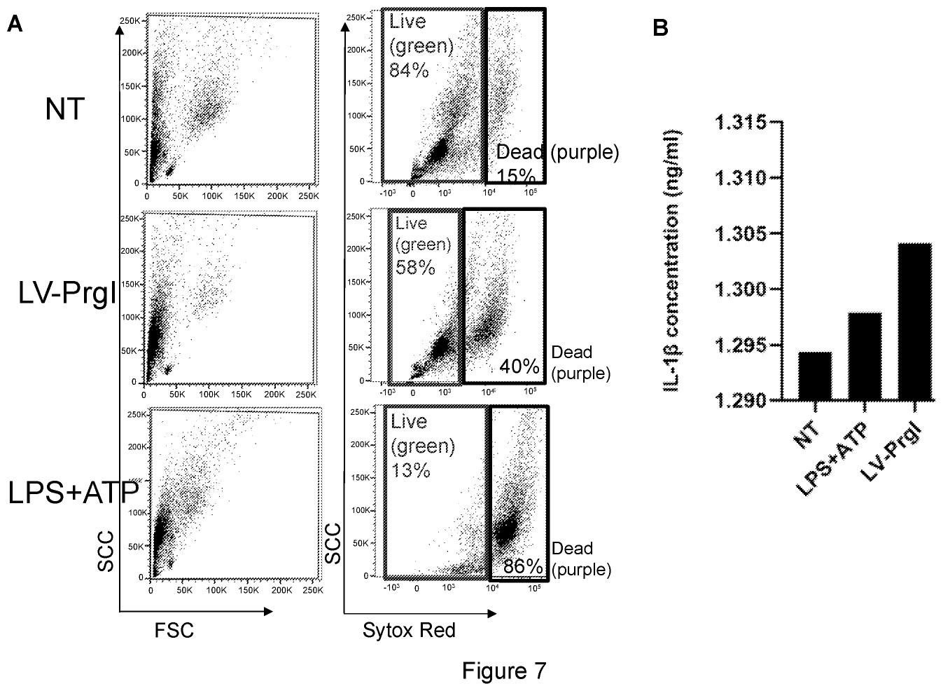

In other experiments, assessment of pyroptosis downstream of inflammasome activation was also conducted by labeling the cells with Sytox Red where only cells that have permeabilized plasma membranes as a result of pyroptosis label with Sytox Red. Here, we confirmed that human DC were undergoing pyroptosis after transduction with PrgI encoding lentiviruses compared to 15% in non-transduced cultures and 86% after a potent inflammasome trigger in the form of LPS+ATP (FIG. 7A). Measurements of IL-1β concentrations in these cultures showed that PrgI elicited higher levels of IL-1β release into the culture supernatants than LPS+ATP (FIG. 7B). This contrasted with the pyroptosis pattern we had observed and indicated that PrgI was more efficient at inducing the release of biologically active IL-1β than the potent trigger LPS+ATP, while it preserved the cells for a longer period of time compared to LPS+ATP (FIGS. 7A and 7B).

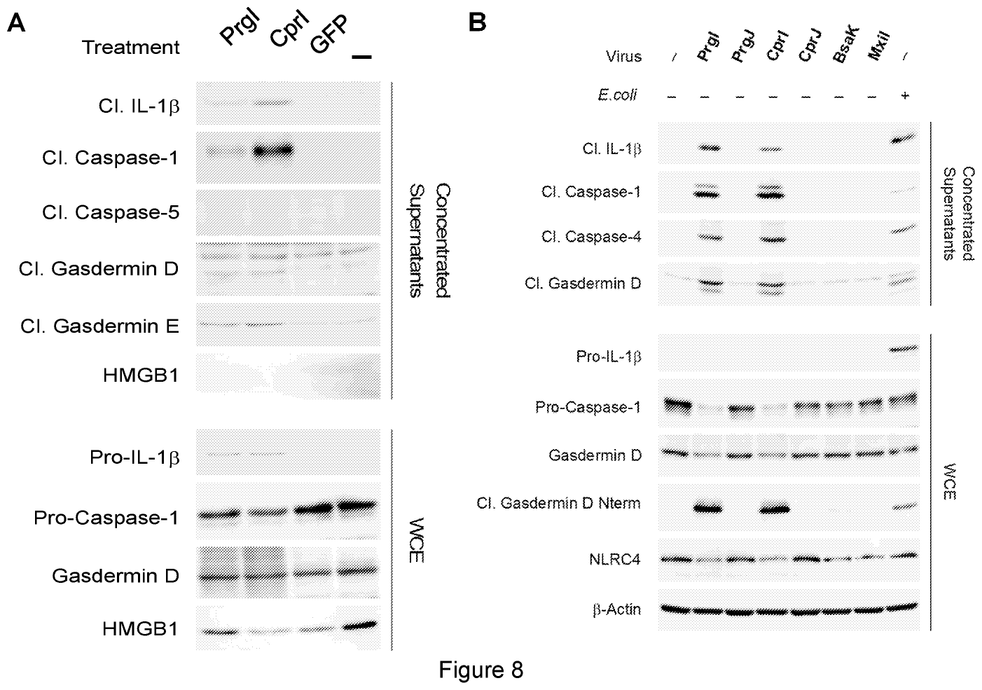

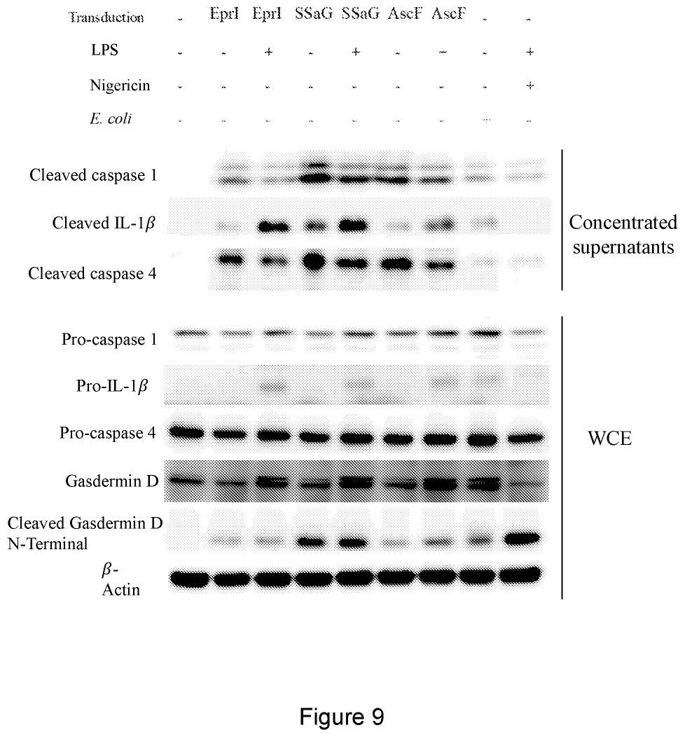

To test whether other Needle or Rod proteins could activate the inflammasome like PrgI, the needle proteins CprI, MxiI, SsaG, EprI and AscF and the Rod proteins CprJ and BsaK were cloned into the lentiviral vector BdLV. Primary human DC cultures were transduced with these recombinant lentiviral vectors on day 7 of culture (as indicated in FIG. 1). 18 hours post transduction, the culture supernatants were concentrated to assess inflammasome activation using Western blot readouts for various effector outputs of active inflammasomes. CprI was as efficient at activating the inflammasome as PrgI evidenced by the detection of cleaved IL-1β, cleaved caspase-1, cleaved Gasdermins D and E in the concentrated culture supernatants (FIGS. 8A and 8B). The levels of HMGB-1 were too low to be detected in these experiments. We included two further parameters of inflammasome activation by immunoprobing for the cleaved form of the noncanonical inflammasome caspase-4 in the concentrated supernatants, and for the N-terminal fragment of Gasdermin D which is retained in WCE upon cleavage of Gasdermin D. We detected cleaved caspase-4 in the concentrated supernatants and accumulation of the cleaved N terminal Gasdermin D fragment in response to PrgI and CprI, consistent with the ability of these two needle proteins to activate the inflammasome in DC (FIG. 8B). Pro-IL-1β levels were no longer detected in the WCE under these conditions reflecting near complete cleavage of pro-IL-1β and its release as the biologically active cleaved form into the concentrated supernatants (FIG. 8B). Like CprI and PrgI, needle proteins SSaG, EprI and AscF were also able to elicit caspase-1 and caspase-4 cleavage and irrespective of treatment with LPS and to levels comparable to those elicited by the positive controls: DC infection with E. coli at 20:1 E. coli to DC ratio, or DC treatment with the combination of 100 ng/ml LPS and 10 μM Nigericin (FIG. 9). For the combination of LPS and lentiviral transduction, LPS was added to DC six hours prior to the addition of recombinant lentiviruses. For the treatment with LPS and Nigericin, Nigericin was added two hours after treatment with LPS for a total treatment time of X hours. CprJ, BsaK, and MxiI did not elicit inflammasome activation as evidenced by the absence of the cleaved forms of IL-1β, caspase-1, caspase-4 and Gasdermin D (FIG. 8B). Together these data show inflammasome activation by 5 different inflammasome agonist proteins of the T3SS family, PrgI, CprI, SSaG, EprI and AscF.

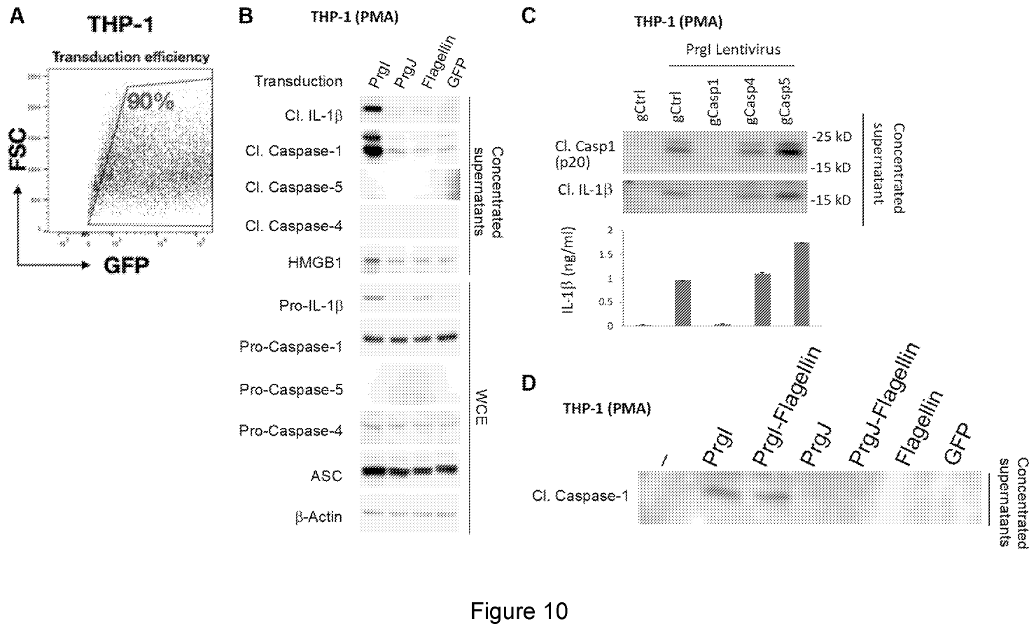

In other experiments, the human monocytic cell line THP-1 was transduced with recombinant BdLV lentiviral vectors encoding PrgI, PrgJ or flagellin. Empty vector served as a control and encoded the GFP reporter alone without PrgI, PrgJ or flagellin. Transduction efficiency was as high as 90% in this cell line as expected (FIG. 10A). Transduced THP-1 cells were differentiated with PMA into macrophages. Assessment of inflammasome activation in these cells at 72 hours post-transduction revealed that PrgI was most efficient at activating the cleavage of IL-1β and caspase-1 and the release of HMGB-1 from cells into the culture supernatants (FIG. 10B). Similar levels of pro-caspase-1, pro-caspase-4 and ASC were detected in the WCE of all transduced THP-1 cells that had been differentiated into macrophages with PMA, whereas the expression of pro-IL-1β was induced best by PrgI and flagellin (FIG. 10B). There was no induction of pro-caspase-5 under any of the conditions (FIG. 10B). Knock-down of caspase-1, caspase-4 or caspase-5 genes by CRISPR/Cas9 and guide RNA specific to these genes (gCasp1, gCasp4, and gCasp5) showed that the cleavage and production of IL-1β production was dependent on caspase-1 and not caspase-4 or caspase-5 (FIG. 10C). This result indicated that PrgI stimulated the release of IL-1β in a manner dependent on the cleavage of caspase-1. Finally, fusion protein expression of PrgI-flagellin was as effective as PrgI in mediating the cleavage of caspase-1 in PMA-differentiated macrophage THP-1 cells (FIG. 10D). Collectively with the results in FIGS. 6-9, these data show that both PrgI can activate the inflammasome in both human macrophages and human dendritic cells.

We established a melanoma tumor model of inducible expression of inflammasome agonist protein (IAP) to test the efficacy of inflammasome agonist proteins (IAP) as an anti-tumor therapy. We transduced B16 melanoma cell line with plasmid pLenti CMV rtTA3 Blast (w756-1) (addgene) that encodes a third-generation lentiviral reverse tetracycline-controlled transactivator (rtTA3) driven from the CMV promoter and conferring resistance of transduced B16 melanoma cells to Blasticidin. B16-rtTA3 cells were established by selection on 8 μg/mL Blasticidin containing Dulbecco's modified Eagle's Medium (DMEM) growth media supplemented with 10% Fetal bovine serum (FBS) (Sigma). B16-rtTA3 were then transduced with a second plasmid pLenti CMVTRE3G eGFP Puro (w819-1). eGFP was excised from the latter plasmid and was replaced with the codon optimized nucleotide sequences—optimized using the OptimumGene algorithm for better mammalian cell expression—and encoding for one of the following IAPs: PrgI, CprI, MxiI, AscF, EprI, and SSaG cloned into the SmaI and XbaI sites. Resultant B16-rtTA3 referred to thereafter as B16-rtTA3-TRE3G-PrgI, B16-rtTA3-TRE3G-PrgI, B16-rtTA3-TRE3G-CprI, B16-rtTA3-TRE3G-CprJ, B16-rtTA3-TRE3G-MxiI, B16-rtTA3-TRE3G-AscF, B16-rtTA3-TRE3G-EprI, or B16-rtTA3-TRE3G-SSaG were selected on both 8 μg/mL Blasticidin and 3 μg/mL Puromycin. This tet-On system enables tetracycline (Doxycyline, Dox) inducible expression of the inflammasome agonist proteins (IAP) PrgI, CprI, MxiI, AscF, EprI, and SSaG in this case, in a Dox inducible manner. Specifically, a tetracycline/Doxycycline tet-On inducible system was used to induce the expression of different Needle proteins in B16 melanoma cells. A tetracycline-controlled transactivator expressing parent B16 melanoma cell line was established by transduction with a lentivirus encoding for a third-generation lentiviral reverse tetracycline-controlled transactivator (rtTA3) driven from the CMV promoter and conferring resistance of transduced cells to Blasticidin. Resultant rtTA3-expressing B16 cells were transduced with a lentivirus encoding for the Needle proteins (MxiI shown in this case) under control of a tet-responsive element (TRE) to which rtTA3 binds upon addition of tetracycline (Doxycycline, Dox) and initiates transcription of the Needle protein. Resultant B16-rtTA3-TRE3G-Needle B16 cells were also resistant to Puromycin

To validate that expression of the IAP elicits cell death of transduced B16 melanoma cells, we treated the cells in culture with 2 μg/mL Dox for a period of 48 hours. Using cultured B16-rtTA3-TRE3G-MxiI cells as a representative, the cells looked healthy comprised mostly of viable cells (98.6%) in the absence of Dox and with or without Puromycin and Blasticidin in the culture, and as visualized by light microscopy and flow cytometry (FIG. 11A). Addition of Dox at either low concentration (0.2 μg/mL) or high concentration (2 μg/mL) elicited cell death as visualized by permeability to 7AAD with a highest fraction of late-stage secondary cell death appearing as Annexin-V⁺ 7AAD⁺ with the higher concentration of Dox (FIG. 11A). It was notable that MxiI was active in eliciting cell death of B16 melanoma cells which are derived from mice showing with a difference in the ability of MxiI to elicit cell death of murine cells and not human dendritic cells. We observed similar results upon the induction of expression of the IAPs AscF, EprI and SSaG in cultured B16 melanoma cells (FIG. 11B). Addition of Dox at either low concentration (0.2 μg/mL) or high concentration (2 μg/mL) elicited cell death as visualized by permeability to 7AAD with a highest fraction of late-stage secondary cell death appearing as Annexin-V⁺ 7AAD⁺ with the high concentration of Dox (FIG. 11C). Induction of IAP expression upon addition of Dox at 2 μg/mL elicited cell death in melanoma cells in culture as measured biochemically by Western blot. Supernatants and whole cell extracts (WCE) of the specific B16-rtTA3-TRE3G-IAP melanoma cells as indicated were collected at 48 hours post treatment with 2 μg/mL Dox. Supernatants were concentrated and probed for the secreted cleaved C-terminal product of Gasdermin D and WCE were probed for uncleaved Gasdermin D. It was notable that all 8 IAP elicited cell death in melanoma cells, even those proteins such as PrgJ and MxiI, which did not elicit inflammasome activation in dendritic cells illustrating cell type and species differences in IAP activity. Collectively, these results show that induction of the expression of eight representative IAP proteins in cultured B16 melanoma cells in vitro elicits melanoma cell death.

We next inoculated C57BL/6J mice subcutaneously with 200,000 B16-rtTA3-TRE3G-PrgI, B16-rtTA3-TRE3G-CprI or B16-rtTA3-TRE3G-MxiI cells expressing PrgI, CprI or MxiI in a Dox inducible manner. Our goal was to elicit reduction of tumor growth upon activation of the inflammasome and induction of B16 melanoma cell pyroptosis when IAP protein expression was induced with administration of 2 mg/mL Dox in the water (FIG. 12A). The site of tumor cell injection was shaved to visualize and measure the volumes of growing tumors at later time points. At day 10, mice were placed on 2 mg/mL Dox water with 0.1 mg/mL sucrose or sucrose alone as a control (no Dox induction of IAP). Mice were taken off Dox or sucrose water on day 13. As a positive control for anti-tumor treatment, we injected mice with 300,000 PMEL T cell receptor transgenic CD8⁺ T cells, specific to the melanoma antigen gp100. The PMEL CD8⁺ T cells were primed with 1 ng/mL interleukin-2 (IL-2)—to elicit activation of their effector function—for 24 hours prior to adoptive transfer intravenously via retroorbital sinus injection on day 12. Digital calipers were used to measure tumor length and width. Tumor volume was calculated using the equation (L×W²)/2. We found that induction of tumor expression of IAP Needle proteins resulted in reduction of tumor volumes and comparably to the positive control PMEL injected mice (FIG. 12B).

We also treated mice by intratumoral injection of 4 mg/kg Dox at day 10 into the base of the tumor at the skin-to-tumor interface. Mice were injected intratumorally for 2 days. Digital calipers were used to measure tumor length and width. Tumor volume was calculated using the equation (L×W²)/2. Tumor volume was calculated using the equation (L×W²)/2. We found that induction of tumor expression of IAP Needle proteins CprI or MxiI by intratumoral injection of Dox significantly impaired tumor growth (FIG. 12C). These results collectively provided proof-of-principle that the expression of IAP proteins in tumors was successful at impairing tumor growth and in some cases significantly lead to regression of tumor volume.

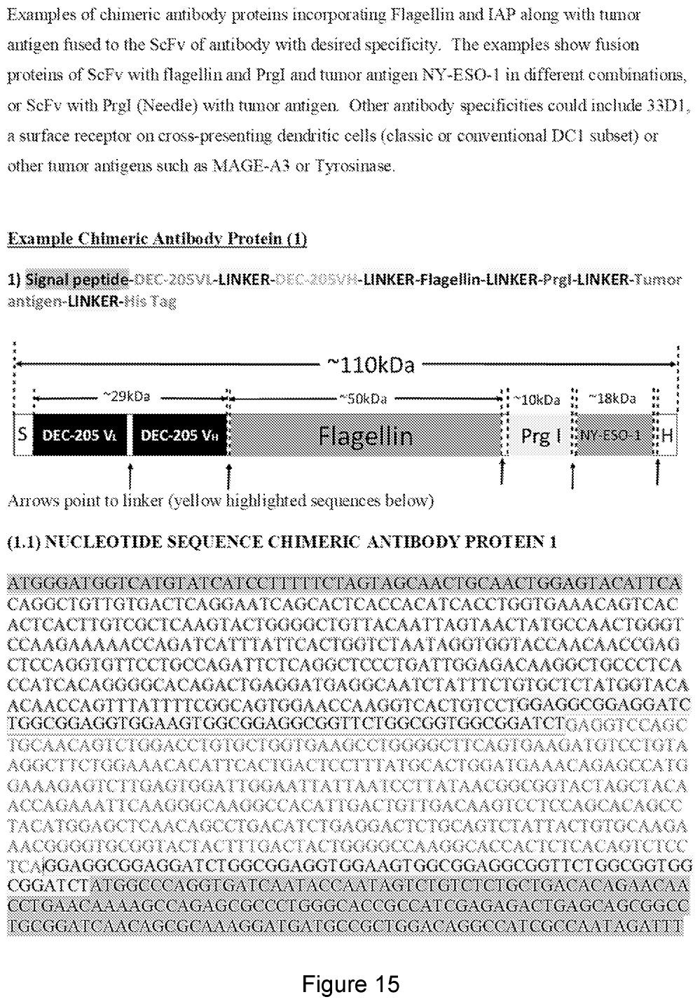

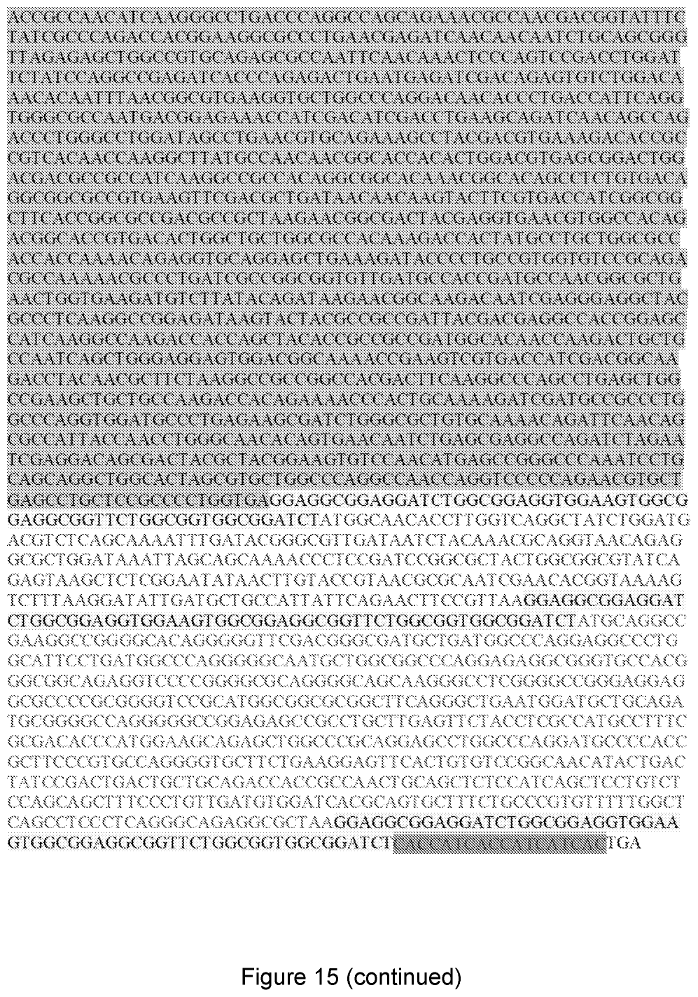

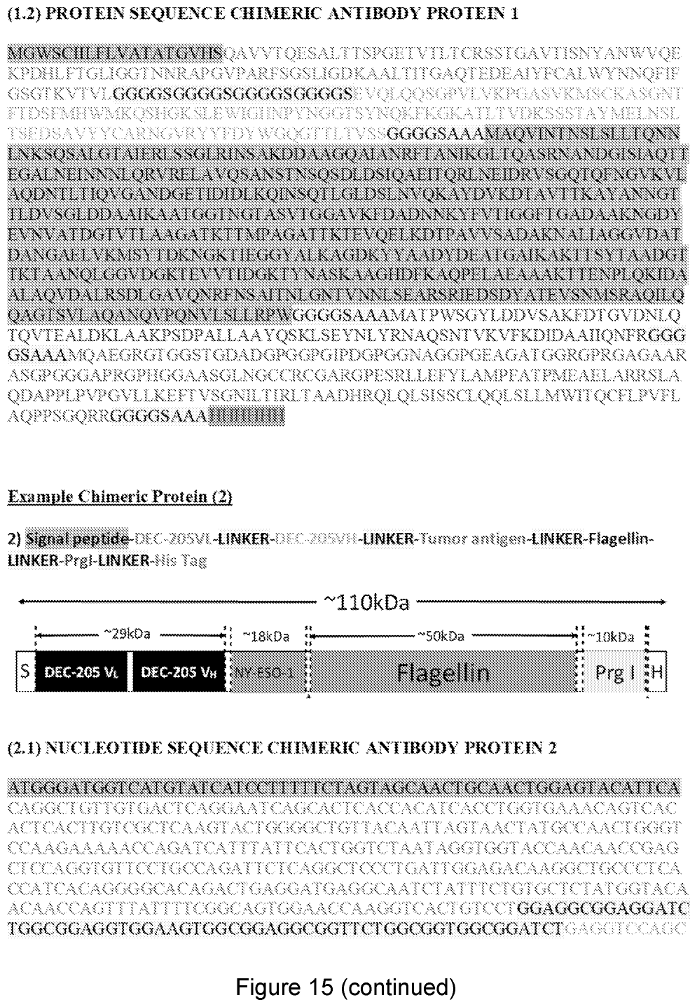

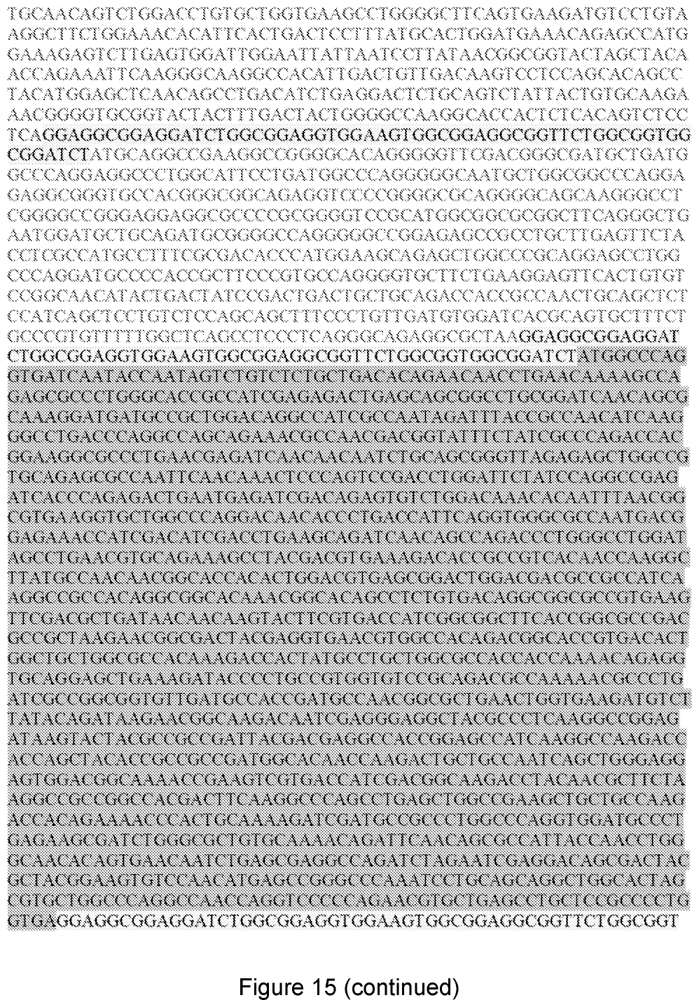

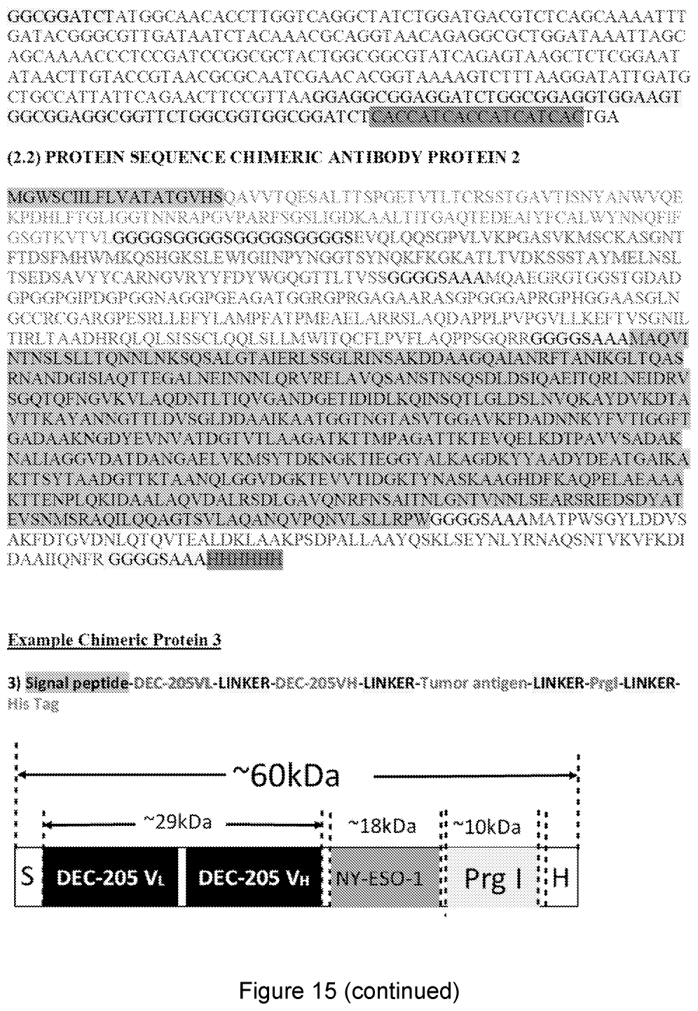

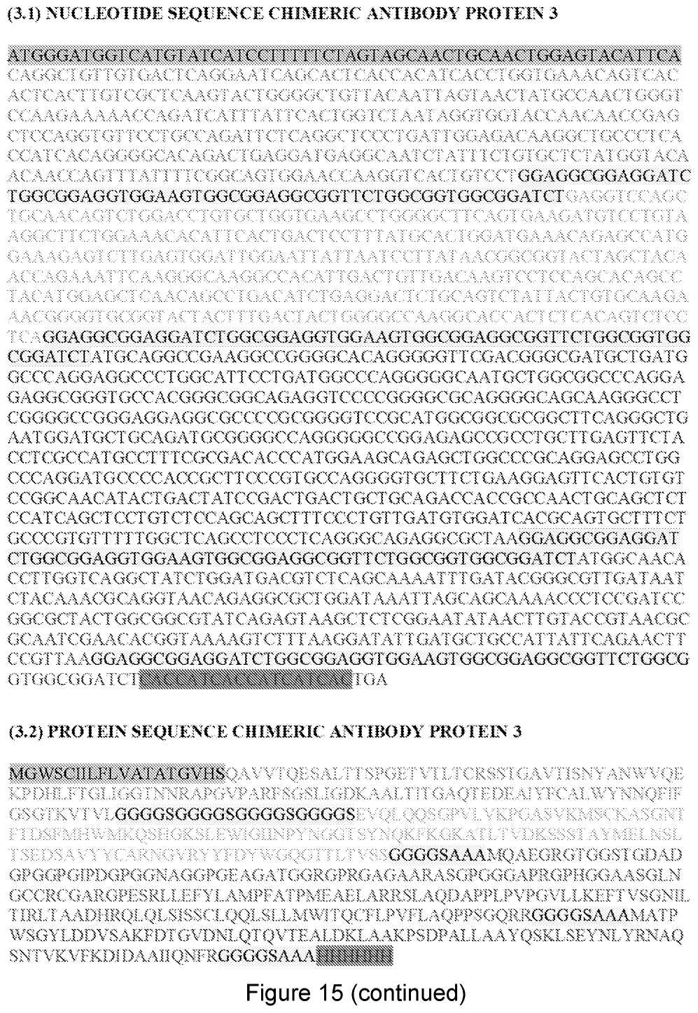

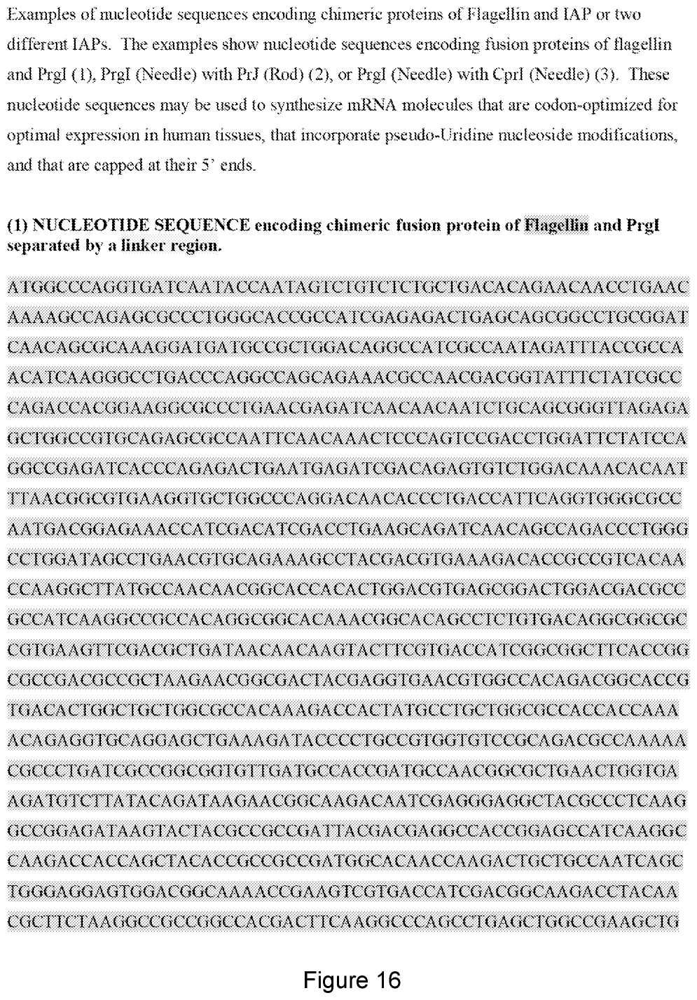

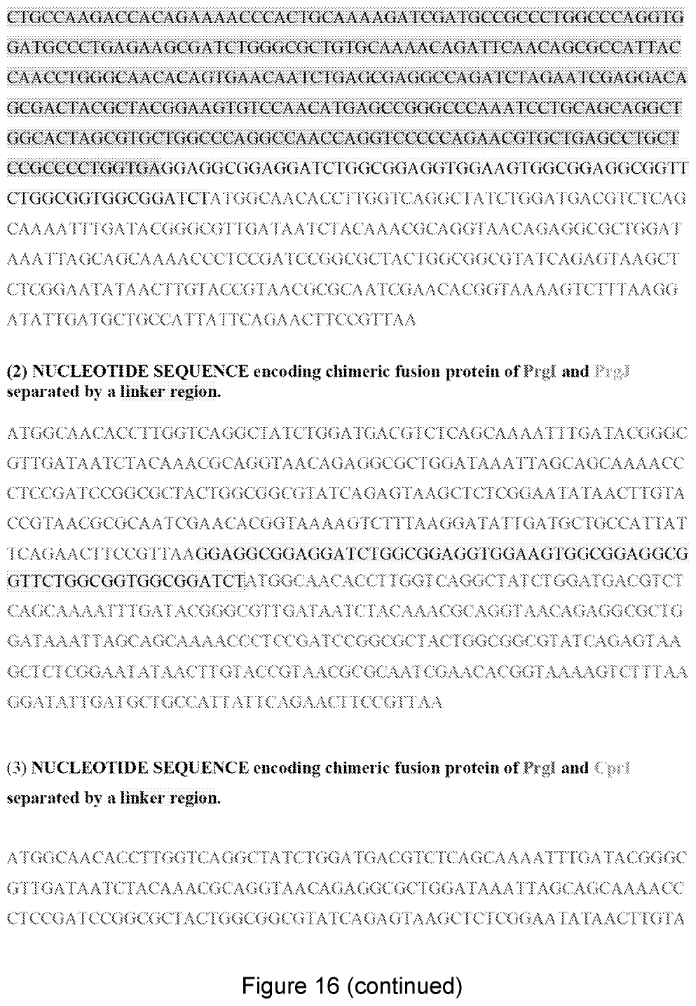

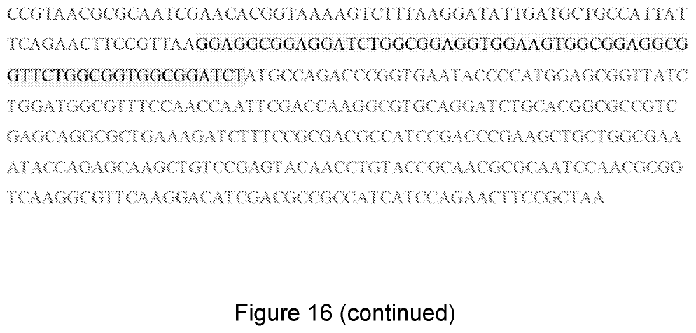

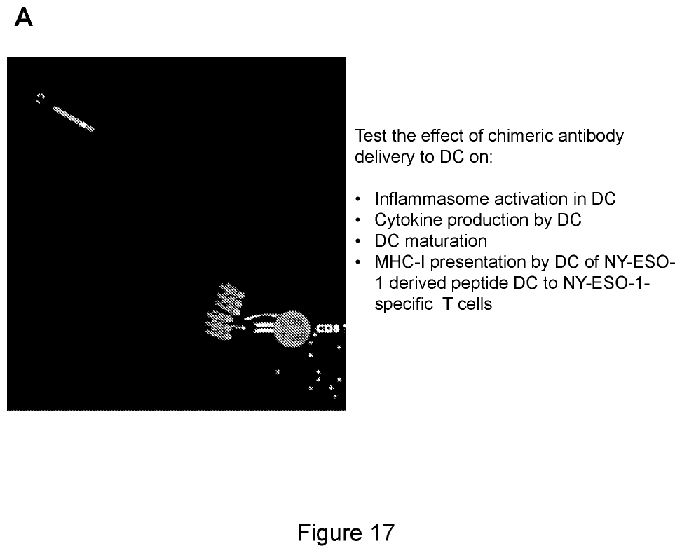

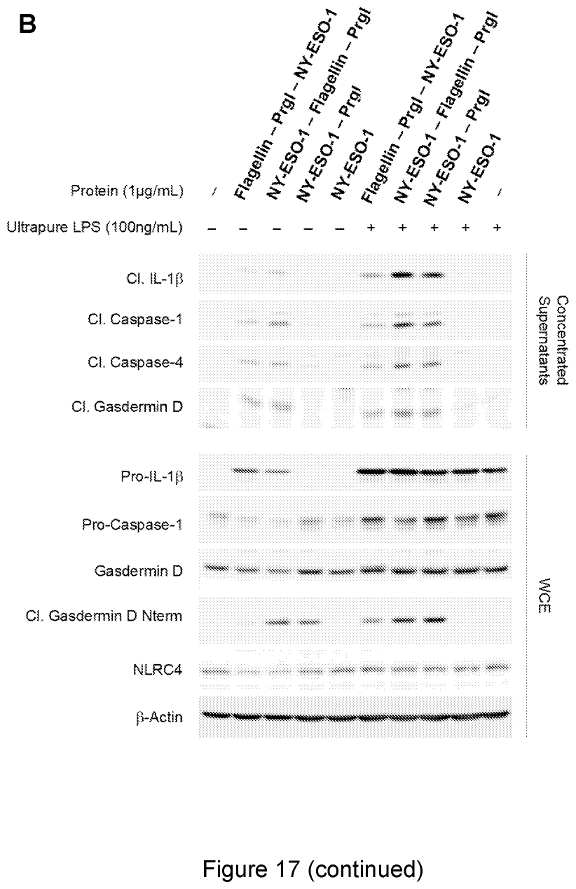

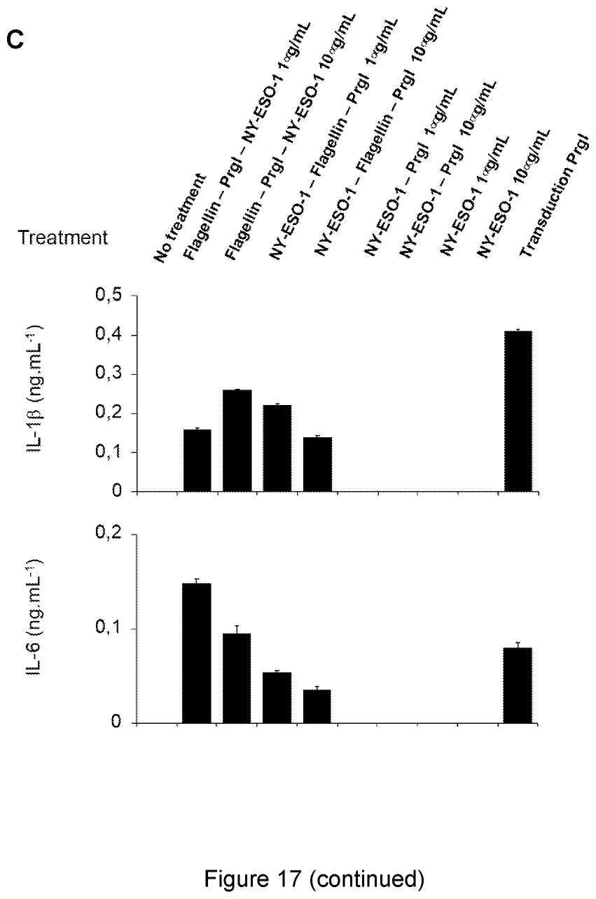

We assessed whether delivery of IAP to dendritic cells in the form of a chimeric fusion antibody targeting the surface molecule DEC-205 would elicit inflammasome activation in DC, production of the inflammasome dependent inflammatory cytokine IL-1β or the inflammasome independent inflammatory cytokine IL-6, and antigen presentation of the co-delivered tumor associated antigen NY-ESO-1 (FIG. 17A). Human DC cultures were treated on day 4 with 1 μg/mL chimeric human antibodies targeting the human DC receptor DEC-205 and comprising a combination of inflammasome agonist and/or flagellin fused to a tumor antigen NY-ESO-1. These chimeric antibodies were: flagellin-PrgI-NY-ESO-1 (chimeric antibody 1), NY-ESO-1-flagellin-PrgI (chimeric antibody 2), NY-ESO-1-PrgI (chimeric antibody 3), or antigen NY-ESO-1 alone as a control (chimeric antibody 4) (as indicated in the schematic in FIG. 15). Another set of DC were treated with the same conditions together with 100 ng/mL LPS to engage TLR4 and achieve the effects of flagellin engagement of TLR5 during treatment with PrgI-NY-ESO-1 (chimeric protein 3) where TLR engagement is not expected. At 24 hours post-treatment or 5 days from the initiation of the human DC differentiation cultures, we harvested the cells for preparation of whole cell extracts to conduct Western blots for various proteins involved in cell death and inflammasome activation. We also harvested the culture supernatants setting aside a small aliquot for conducting ELISAs for measurement of IL-Iβ protein and IL-6, and we concentrated the proteins within the rest of the supernatants to conduct Western blots for detection of the cleaved forms of IL-1β and caspases 1 and 4 as well as cleavage of Gasdermin D. We detected the pro-forms caspase-1 and Gasdermin D in the WCE under all conditions of treatment irrespective of treatment, but the pro-forms of IL-1β were detected only under conditions of TLR engagement either by flagellin (engagement of TLR5) or LPS (engagement of TLR4) (FIG. 17B). Notably, DC treatment with flagellin-PrgI-NY-ESO-1 (chimeric antibody 1) or NY-ESO-1-flagellin-PrgI (chimeric antibody 2) elicited inflammasome activation as evidenced by the detection of the cleaved forms of IL-1β, caspase-1, caspase-4 and Gasdermin D in the concentrated supernatants and the cleaved N-terminal fragment of Gasdermin D in the WCE, and irrespective of co-treatment with LPS (FIG. 17B). DC treatment with NY-ESO-1-PrgI (chimeric antibody 3) also elicited inflammasome activation but only when DC were also treated with LPS, indicative of the need for inflammasome priming when PrgI is delivered to DC in the form of a chimeric antibody (FIG. 17B). Concordant with these results, treatment of DC with two concentrations 1 and 10 μg/mL of flagellin-PrgI-NY-ESO-1 (chimeric antibody 1) or NY-ESO-1-flagellin-PrgI (chimeric antibody 2) led to the secretion of IL-1β as well as IL-6 protein in the supernatants as measured by ELISA (FIG. 17C). We used IL-6 as a marker of TLR engagement/inflammasome priming and found that only those chimeric antibodies that incorporated flagellin to engage TLR5 elicited IL-6 production (FIG. 17C). These results were concordant with the ability of these same chimeric antibodies 1 and 2 to elicit pro-IL-1β expression independently of treatment with LPS (FIG. 17B). Together, these results show that delivery of needle protein to DC along with a TLR agonist flagellin within the framework of a chimeric antibody to a surface receptor such as DEC-205 is an efficient method of eliciting effector functions of the inflammasome.

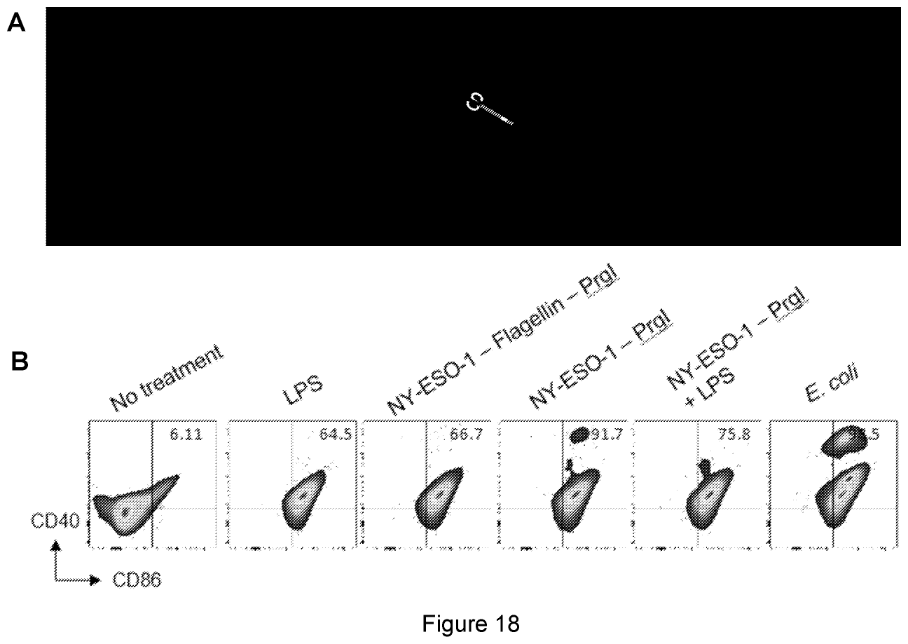

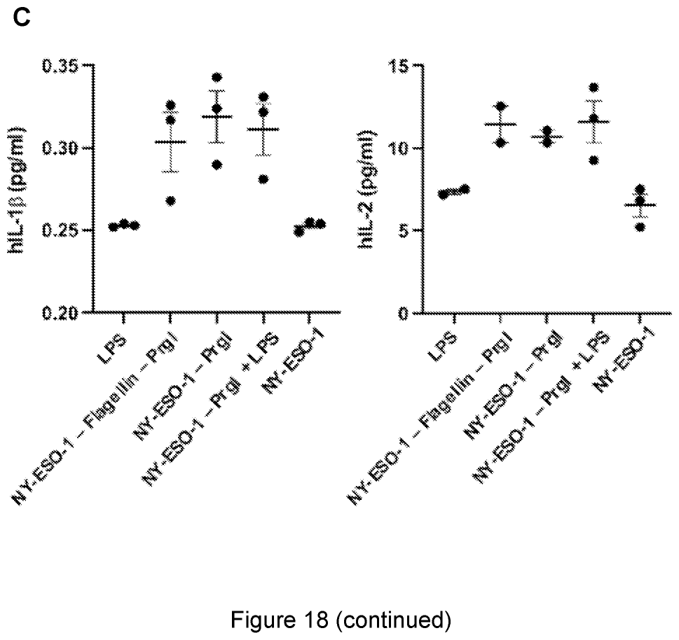

To determine whether delivery of a tumor associated antigen NY-ESO-1 in the framework of the chimeric antibodies flagellin-PrgI-NY-ESO-1 (chimeric antibody 1), NY-ESO-1-flagellin-PrgI (chimeric antibody 2), NY-ESO-1-PrgI (chimeric antibody 3), or antigen NY-ESO-1 alone as a control (chimeric antibody 4) (as indicated in the schematic in FIG. 15), we isolated human CD14⁺ monocytes from peripheral blood mononuclear cells (PBMC) and differentiated them into human DC by culture with human GM-CSF and IL-15 (FIG. 18A). On day 8, DC were treated with 100 ng/mL LPS alone or the following conditions: NY-ESO-1-flagellin-PrgI (chimeric antibody 2), NY-ESO-1-PrgI (chimeric antibody 3), NY-ESO-1-PrgI (chimeric antibody 3) with 100 ng/mL LPS to elicit priming through TLR4 as discussed above, or chimeric antibody NY-ESO-1 alone as a control (chimeric antibody 4). Some wells of cultured DC were also treated with Escherichia coli at a ratio of 1DC:20 E. coli. Stimulation with LPS and E. coli also served as a positive control for DC maturation. DCs were incubated with these treatments and stimuli for 6 hours. Treatment with the chimeric antibodies 2, 3, and 4 resulted in DC maturation as evidenced by the increase in expression of the T cell co-stimulatory molecules CD40 and CD86 at 6 hours post treatment and to levels comparable to those elicited by the positive control stimuli LPS and E. coli (FIG. 18B). At 6 hours post treatment with the chimeric proteins or LPS, 200,000 human DC were cultured with NY-ESO-1 TCR expressing Jurkat T cell line at a 1:1 ratio for a period of 72 hours. Measurement of the cytokine IL-1β in culture supernatants showed that all chimeric proteins containing the IAP PrgI elicited IL-1β production by DC while chimeric antibody NY-ESO-1 alone did not do so (FIG. 18C). As expected, no IL-1β was produced in response to LPS because of the lack of an inflammasome trigger. Concordant with these results, NY-ESO-1 TCR Jurkat T cells produced the T cell growth factor IL-2 only in response to DC that had been treated with NY-ESO-1-flagellin-PrgI (chimeric antibody 2), NY-ESO-1-PrgI (chimeric antibody 3), NY-ESO-1-PrgI (chimeric antibody 3) with 100 ng/mL LPS and not chimeric antibody NY-ESO-1 alone (FIG. 18C). Together these results demonstrate that IAP delivery to DC through the receptor DEC-205 leads to inflammasome activation, IL-1β production, DC maturation and presentation of a tumor associated antigen to antigen-specific T cells.

Although the present disclosure has been described using specific embodiments and examples, routine modifications will be apparent to those skilled in the art and such modifications are intended to be within the scope of the disclosure and the claims.

1. A method comprising introducing an Inflammasome Agonist Protein (IAP) that is a Needle or Rod protein into an individual in need thereof to thereby stimulate an immune response in the individual.

2. The method of claim 1, wherein:
i) the IAP is a component of a fusion protein; or
ii) the IAP is encoded by a polynucleotide that is introduced into cells of the individual such that the IAP is expressed within the cells, and wherein the polynucleotide is a optionally an mRNA, and wherein the mRNA is optionally present within liposomal nanoparticles.

3. The method of claim 1, wherein a Toll Like Receptor (TLR) agonist is also administered to the individual, and wherein the TLR agonist is not a component of the fusion protein.

4. The method of claim 2, wherein i) is introduced, wherein the fusion protein further comprises a Toll Like Receptor (TLR) agonist, or a cancer antigen, or a combination thereof.

5. The method of claim 2, wherein the fusion protein is administered, and wherein the fusion protein comprises a binding partner that binds with specificity to a dendritic cell surface marker, or a binding partner that binds with specificity to a cancer cell surface marker, and wherein the binding partner comprises an antibody heavy and antibody light chain.

6. The method of claim 5, wherein the binding partner comprises a single-chain variable fragment (scFv).

7. The method of claim 2, wherein ii) is introduced, and wherein polynucleotide further encodes a Toll Like Receptor (TLR) agonist, a cancer antigen, a binding partner that binds with specificity to a dendritic cell surface marker, a binding partner that binds with specificity to a cancer cell surface marker, or a combination thereof.

8. The method of claim 7, wherein the binding partner is present and comprises an antibody heavy and antibody light chain.

9. The method of claim 8, wherein the binding partner comprises a single-chain variable fragment (scFv).

10. The method of claim 1, wherein the IAP is selected from the group consisting of PrgI, CprI, EprI, SSaG, AscF, and a combination thereof.

11. The method of claim 10, wherein a TLR agonist is introduced into the individual, and wherein the TLR agonist comprises flagellin or a TLR5-binding flagellin derivative.

12. The method of claim 10, wherein the IAP is introduced into dendritic cells that activate T cells that participate in inhibiting growth of and/or killing cancer cells that express a cancer antigen, and wherein the cancer antigen is also optionally administered to the individual.

13. The method of claim 12, wherein the T cells are CD8+ T cells.

14. The method of claim 10, wherein a fusion protein or a polynucleotide encoding the fusion is introduced, and wherein the fusion protein comprises flagellin as the TLR agonist and an scFv that binds with specificity to a dendritic cell surface marker.

15. The method of claim 14, wherein the TLR agonist comprises a TLR5 agonist.

16. The method of claim 14, wherein subsequent to expression of the IAP the growth of the cancer cells in the individual is inhibited or the cancer cells die.

17. The method of claim 16, wherein the cancer cells die by pyroptosis.

18. A fusion protein comprising an Inflammasome Agonist Protein (IAP) that is a Needle or Rod protein, the fusion protein further comprising at least one of a cancer antigen, a Toll Like Receptor (TLR) agonist, or a binding partner that binds with specificity to a dendritic cell surface marker, or a binding partner that binds with specificity to a cancer cell surface marker.

19. An expression vector or mRNA encoding the fusion protein of claim 18.

20. A pharmaceutical formulation comprising a polynucleotide encoding an Inflammasome Agonist Protein (IAP) that is a Needle or Rod protein.

21. The pharmaceutical formulation of claim 20, wherein the polynucleotide comprises an mRNA.

22. The pharmaceutical formulation of claim 21, wherein the mRNA is present within liposomal nanoparticles.

23. The pharmaceutical formulation of claim 20, wherein the IAP is selected from the group consisting of PrgI, CprI, EprI, SSaG, AscF, and a combination thereof.
